(19) United States
(12) Patent Application Publication
(10) Pub. No.: US 2016/0177381 A1
(43) Pub. Date: Jun. 23, 2016

(54) Base-Pair Specific Inter-Strand Locks for Genetic and Epigenetic Detection

(71) Applicant: THE CURATORS OF THE UNIVERSITY OF MISSOURI, Columbia, MO (US)

(72) Inventors: Li-Qun Gu, Columbia, MO (US); Yong Wang, Columbia, MO (US); Kai Tian, Columbia, MO (US)

(21) Appl. No.: 14/910,198

(22) PCT Filed: Aug. 5, 2014
(86) PCT No.: PCT/US14/49802

§ 371 Date: Feb. 4, 2016

Related U.S. Application Data
(60) Provisional application No. 61/958,747, filed on Aug. 5, 2013.

(51) Int. Cl. C12Q 1/68

(52) U.S. Cl. CPC C12Q 1/6827

(57) ABSTRACT

A versatile detection method is disclosed that utilizes a base-pair-specific inter-strand lock for genetic and epigenetic detection. Reagents, devices, etc., for implementing the method have also been discovered and/or developed. In certain embodiments, compounds have been identified to be able to specifically bind certain mismatched base pairs including T-T, U-T, and C-C base pair mismatches using either Hg²⁺ or Ag⁺. Such binding can strengthen the base-pair hybridization in orders of magnitude, forming a so-called reversible inter-strand lock that can greatly stabilize double-stranded nucleic acid fragments.

### CROSS REFERENCE TO RELATED APPLICATIONS

This International patent application claims the benefit of U.S. Provisional Patent Application No. 61/958,747, which was filed Aug. 5, 2013 and is incorporated herein by reference in its entirety.

### STATEMENT REGARDING FEDERALLY SPONSORED RESEARCH

This invention was made with Government support under Grant No. 5R01GM079613 awarded by the National Institutes of Health. The Government has certain rights in the invention.

### SEQUENCE LISTING STATEMENT

A sequence listing is contained in the file named “134248_SEQ_LIST_ST25.txt” which is 7,120 bytes (measured in MS-Windows) and was created on Aug. 5, 2014, and comprising 33 nucleotide sequences and is electronically filed herewith and is incorporated herein by reference.

### BACKGROUND

Gene expression is not only controlled by the DNA sequence itself, but by epigenomic factors, i.e., chemically modified DNAs and chromatin proteins that causes inherited alteration of gene expression without changing DNA sequences. DNA methylation is one of the most commonly occurring epigenetic events in human genome. It is a covalent addition of a methyl group to the cytosine ring by DNA methyltransferases. Most DNA methylation occurs in CpG dinucleotides (5′-CG-3′), and over half of all the human genes have a CG rich stretch around promoters and/or the first exon regions, called CpG islands. They are free of methylation in normal somatic cells, but many CpG islands in cancer cells are aberrantly methylated to cause gene silencing. Since abnormal DNA methylation in promoter CpG islands is a hall marker of all types of cancers and is chemically stable, it has emerged as a potential biomarker for assessing cancer risk, early detection, prognosis and predicting therapeutic responses.

Many methods have been developed for the examination of DNA methylation, such as bisulfite sequencing CpG island microarray, quantitative methylation-specific PCR (MSP) and mass spectrometry. High-throughput microarrays and next generation sequencing are capable of analyzing genome-wide patterns of DNA methylation, and led to the discovery of many novel methylated genes in various types of tumors. Other less-expensive and highly-sensitive methods, such as quantitative methylation-specific PCR (MethyLight) and combined bisulfite restriction analysis (COBRA) are useful in target validation or in a clinical diagnostic setting for detection of specific gene methylation in cancer and other diseases. A cornerstone step in these assays is bisulfite treatment of DNA that introduces specific changes in the DNA strands. The changes depend on the methylation status of individual cytosine residues, yielding single nucleotide resolution information about the methylation status of a DNA segment. Recently, new techniques that integrate single-molecule and nanotechnology have emerged for base-specific determination of methylation status. Many of these reported methods, however, are not highly quantitative. The detection employ expensive instrument, and the procedure is laborious, involving complex chemical labeling and amplification. These limit their applications in the clinical setting.

Cytosine (C) modifications such as 5-methylcytosine (mC) and 5-hydroxymethylcytosine (hmC) are important epigenetic markers associated with gene expression and tumorigenesis. However, bisulfite conversion, the gold standard methodology for mC mapping, cannot distinguish mC and hmC bases. Studies have demonstrated hmC detection via peptide recognizing, enzymes, fluorescence and hmC-specific antibodies, nevertheless, a method for directly discriminating C, mC and hmC bases without labeling, modification and amplification is still missing.

### SUMMARY

Certain embodiments are drawn to methods of detecting a thymine-thymine (T-T) base pair mismatch or a uracil-thymine (U-T) base pair mismatch in an at least partially double-stranded oligonucleotide (ds-oligonucleotide). Such methods comprise reversibly binding Hg²⁺ to the base pair mismatch. This binding increases the hybridization stability of the ds-oligonucleotide in comparison to its hybridization stability in the absence of Hg²⁺ reversible binding. In certain embodiments, the T-T or U-T base pair mismatch is within a contiguous region of at least 10 nucleotides that are hybridized in the ds-oligonucleotide. The T-T or U-T base pair mismatch may be detected by detecting the increased hybridization stability of the ds-oligonucleotide.

In certain embodiments of detecting a T-T base pair mismatch or a U-T base pair mismatch: the method comprises hybridizing a first single-stranded oligonucleotide to a second single stranded oligonucleotide to form an at least partially ds-oligonucleotide comprising the T-T or U-T base pair mismatch and contacting the ds-oligonucleotide with Hg²⁺; the Hg²⁺ is provided by the addition of HgCl₂; either a first single-stranded oligonucleotide or a second single-stranded oligonucleotide comprises a tag domain comprising a polydeoxycytosine covalently bound to the 3′-end, the 5′-end, or both the 3′-end and the 5′-end of the hybridizing region; the tag domain is poly(dC)₃₀; at least 6, at least 7, at least 8, or at least 9 of the base-pairings within the contiguous hybridized region of at least 10 nucleotides are non-mismatched base-pairings; the base pair mismatch in the hybridized region is a T-T mismatch; the base pair mismatch in the hybridized region is a U-T mismatch; at least one of a first ss-oligonucleotide and a second ss-oligonucleotide comprises an oligonucleotide from about 10, 12, 14, 16, or 19 to about 20, 25, 30, 40, 50, 60, 100 or more nucleotides in length; the hybridized region is a contiguous region of between about 10, 12, 14, or 16 to about 20, 25, 30, 40, 50, 60, 100, or more nucleotides; the increase in hybridization stability of the ds-oligonucleotide is detected with a nanopore, PCR, gold nanoparticle, horseradish peroxidase, atomic force microscope, or immuo-PCR; the increased hybridization stability of the ds-oligonucleotide is detected with a nanopore; and/or nanopore detection of the increase in hybridization stability of the ds-oligonucleotide comprises: (a) applying a voltage to a sample containing the ds-oligonucleotide in a cis compartment of a duel chamber nanopore system, the voltage sufficient to drive translocation of the hybridized ds-oligonucleotide through a nanopore of said system by an unzipping process; and (b) analyzing an electrical current pattern in the nanopore system over time, wherein the increased hybridization stability of the ds-oligonucleotide in the presence of reversible Hg²⁺ binding produces an electrical current pattern that is different and distinguishable from an electrical current pattern produced by the ds-oligonucleotide in the absence of Hg²⁺.

Certain embodiments are drawn to methods of determining whether a cytosine residue in a target single-stranded oligonucleotide (ss-oligonucleotide) or in a target strand of a double-stranded oligonucleotide (ds-oligonucleotide) is a methylated cytosine residue or an un-methylated cytosine residue. Such methods comprise treating the target ss-oligonucleotide or target strand of the ds-oligonucleotide with bisulfite to convert an un-methylated cytosine residue, if present, to a uracil residue but wherein said treatment does not convert a methylated cytosine residue, if present, to a uracil residue. The methods also comprise hybridizing the bisulfite treated target ss-oligonucleotide or bisulfite treated target strand of the ds-oligonucleotide and a probe molecule to form an at least partially double-stranded target/probe oligonucleotide that comprises a thymine residue base pair mismatched with the converted uracil residue, if present, from the target ss-oligonucleotide or target strand of the ds-oligonucleotide or that comprises a thymine residue base pair mismatched with the un-converted methylated cytosine residue, if present, from the target ss-oligonucleotide or target strand of the ds-oligonucleotide. In certain embodiments, the uracil-thymine base pair mismatch or the methylated cytosine-thymine base pair mismatch is within a contiguous region of at least 10 nucleotides that are hybridized in the target/probe oligonucleotide. The methods also comprise contacting the target/probe oligonucleotide with Hg²⁺, wherein Hg²⁺ reversibly binds the uracil-thymine base pair mismatch but not the methylated cytosine-thymine mismatch. The methods also comprise detecting the presence or absence of the reversible binding of Hg²⁺, wherein the presence indicates that the cytosine residue in the target ss-oligonucleotide or in the target strand of the ds-oligonucleotide was un-methylated and the absence indicates that the cytosine residue in the target ss-oligonucleotide or in the target strand of the ds-oligonucleotide was methylated.

In certain embodiments of determining whether a cytosine residue in a target single-stranded oligonucleotide (ss-oligonucleotide) or in a target strand of a double-stranded oligonucleotide (ds-oligonucleotide) is a methylated cytosine residue or an un-methylated cytosine residue: at least 6, at least 7, at least 8, or at least 9 of the base-pairings within the contiguous hybridized region of at least 10 nucleotides are non-mismatched base-pairings; at least the target ss-oligonucleotide or target strand of the ds-oligonucleotide, or probe molecule comprises an oligonucleotide from about 10, 12, 14, 16, or 19 to about 20, 25, 30, 40, 50, 60, 100 or more nucleotides in length; the probe molecule comprises a tag domain comprising a polydeoxycytosine covalently bound to the 3′-end, the 5′-end, or both the 3′-end and the 5′-end of the hybridizing region; the tag domain is poly(dC)₃₀; the Hg²⁺ is provided by the addition of HgCl₂; the method further comprises detecting the increase in the hybridization stability of the target/probe oligonucleotide; the increase in hybridization stability of the target/probe oligonucleotide is detected with a nanopore, PCR, gold nanoparticle, horseradish peroxidase, atomic force microscope, or immuo-PCR; the increased hybridization stability of the ds-oligonucleotide is detected with a nanopore; the increase is detected using a nanopore; and/or the nanopore detection of the increase in hybridization stability of the ds-oligonucleotide comprises (a) applying a voltage to a sample containing the ds-oligonucleotide in a cis compartment of a duel chamber nanopore system, the voltage sufficient to drive translocation of the hybridized ds-oligonucleotide through a nanopore of said system by an unzipping process; and (b) analyzing an electrical current pattern in the nanopore system over time, wherein the increased hybridization stability of the ds-oligonucleotide in the presence of reversible Hg²⁺ binding produces an electrical current pattern that is different and distinguishable from an electrical current pattern produced by the ds-oligonucleotide in the absence of Hg²⁺.

Certain embodiments are drawn to methods of increasing the hybridization stability of an at least partially double-stranded oligonucleotide comprising a T-T or a U-T base pair mismatch. Such methods comprise reversibly binding Hg²⁺ to the base pair mismatch, thereby increasing the hybridization stability of the ds-oligonucleotide. In certain embodiments, the T-T or U-T base pair mismatch is within a contiguous region of at least 10 nucleotides that are hybridized in the ds-oligonucleotide.

In certain embodiments of increasing the hybridization stability of an at least partially double-stranded oligonucleotide (ds-oligonucleotide) comprising a thymine-thymine (T-T) or a uracil-thymine (U-T) base pair mismatch: the method comprises hybridizing a first single-stranded oligonucleotide to a second single stranded oligonucleotide to form the at least partially ds-oligonucleotide comprising the T-T or U-T base pair mismatch and contacting the ds-oligonucleotide with Hg²⁺; at least 6, at least 7, at least 8, or at least 9 of the base-pairings within the contiguous hybridized region of at least 10 nucleotides are non-mismatched base-pairings; at least one of the first ss-oligonucleotide and the second ss-oligonucleotide comprises an oligonucleotide from about 10, 12, 14, 16, or 19 to about 20, 25, 30, 40, 50, 60, 100 or more nucleotides in length; the first single-stranded oligonucleotide or the second single-stranded oligonucleotide comprises a tag domain comprising a polydeoxycytosine covalently bound to the 3′-end, the 5′-end, or both the 3′-end and the 5′-end of the hybridizing region; the tag domain is poly(dC)₃₀; the Hg²⁺ is provided by the addition of HgCl₂; the method further comprises detecting the increase in the hybridization stability of the target/probe oligonucleotide; the increase in hybridization stability of the target/probe oligonucleotide is detected with a nanopore, PCR, gold nanoparticle, horseradish peroxidase, atomic force microscope, or immuo-PCR; and/or the increased hybridization stability of the ds-oligonucleotide is detected with a nanopore. In certain embodiments of increasing the hybridization stability of an at least partially double-stranded oligonucleotide (ds-oligonucleotide) comprising a thymine-thymine (T-T) or a uracil-thymine (U-T) base pair mismatch the increase is detected using a nanopore, and the nanopore detection of the increase in hybridization stability of the ds-oligonucleotide comprises: (a) applying a voltage to a sample containing the ds-oligonucleotide in a cis compartment of a duel chamber nanopore system, the voltage sufficient to drive translocation of the hybridized ds-oligonucleotide through a nanopore of said system by an unzipping process; and (b) analyzing an electrical current pattern in the nanopore system over time, wherein the increased hybridization stability of the ds-oligonucleotide in the presence of reversible Hg²⁺ binding produces an electrical current pattern that is different and distinguishable from an electrical current pattern produced by the ds-oligonucleotide in the absence of Hg²⁺.

Certain embodiments are drawn to methods of detecting a cytosine-cytosine (C-C) base pair mismatch or a methylcytosine-cytosine (mC-C) in an at least partially double-stranded oligonucleotide (ds-oligonucleotide). Such methods comprise reversibly binding Ag⁺ to the base pair mismatch. This binding increases the hybridization stability of the ds-oligonucleotide in comparison to its hybridization stability in the absence of Ag⁺ reversible binding. In certain embodiments, the C-C or mC-C base pair mismatch is within a contiguous region of at least 10 nucleotides that are hybridized in the ds-oligonucleotide. The methods comprise detecting the increased hybridization stability of the ds-oligonucleotide thereby detecting the C-C or mC-C base pair mismatch.

In certain embodiments of detecting a cytosine-cytosine (C-C) base pair mismatch or a methylcytosine-cytosine (mC-C) in an at least partially double-stranded oligonucleotide (ds-oligonucleotide): the increase in hybridization stability of the ds-oligonucleotide is detected with a nanopore, PCR, gold nanoparticle, horseradish peroxidase, atomic force microscope, or immuo-PCR; the increased hybridization stability of the ds-oligonucleotide is detected with a nanopore; at least 6, at least 7, at least 8, or at least 9 of the base-pairings within the contiguous hybridized region of at least 10 nucleotides are non-mismatched base-pairings; the method comprises hybridizing a first single-stranded oligonucleotide to a second single stranded oligonucleotide to form the at least partially ds-oligonucleotide comprising the C-C or mC-C base pair mismatch and contacting the ds-oligonucleotide with Ag⁺; the first single-stranded oligonucleotide or the second single-stranded oligonucleotide comprises a tag domain comprising a polydeoxycytosine covalently bound to the 3′-end, the 5′-end, or both the 3′-end and the 5′-end of the hybridizing region; the tag domain is poly(dC)₃₀; the base pair mismatch in the hybridized region is a cytosine-cytosine mismatch; the base pair mismatch in the hybridized region is a methylcytosine-cytosine mismatch; at least one of the first ss-oligonucleotide and the second ss-oligonucleotide comprises an oligonucleotide from about 10, 12, 14, 16, or 19 to about 20, 25, 30, 40, 50, 60, 100 or more nucleotides in length; and/or the hybridized region is a contiguous region of between about 10, 12, 14, or 16 to about 20, 25, 30, 40, 50, 60, 100, or more nucleotides. In certain embodiments of detecting a cytosine-cytosine (C-C) base pair mismatch or a methylcytosine-cytosine (mC-C) in an at least partially double-stranded oligonucleotide (ds-oligonucleotide), nanopore detection of the increase in hybridization stability of the ds-oligonucleotide comprises: (a) applying a voltage to a sample containing the ds-oligonucleotide in a cis compartment of a duel chamber nanopore system, the voltage sufficient to drive translocation of the hybridized ds-oligonucleotide through a nanopore of said system by an unzipping process; and (b) analyzing an electrical current pattern in the nanopore system over time, wherein the increased hybridization stability of the ds-oligonucleotide in the presence of reversible Ag⁺ binding produces an electrical current pattern that is different and distinguishable from an electrical current pattern produced by the ds-oligonucleotide in the absence of Ag⁺.

Certain embodiments are drawn to methods of discriminating between a cytosine residue, a methylcytosine residue, and a hydroxymethylcytosine residue in a target single-stranded oligonucleotide (ss-oligonucleotide) or in a target strand of a double-stranded oligonucleotide (ds-oligonucleotide). Such methods comprise hybridizing the target ss-oligonucleotide or target strand of the ds-oligonucleotide and a probe molecule to form an at least partially double-stranded target/probe oligonucleotide that comprises a cytosine residue from the probe molecule base pair mismatched with a cytosine from the target ss-oligonucleotide or target strand of the ds-oligonucleotide, if present, a cytosine residue from the probe molecule base pair mismatched with a methylcytosine residue from the target ss-oligonucleotide or target strand of the ds-oligonucleotide, if present, or a cytosine residue from the probe molecule base pair mismatched with a hydroxymethylcytosine residue from the target ss-oligonucleotide or target strand of the ds-oligonucleotide, if present. In certain embodiments, the cytosine-cytosine mismatch, the cytosine-methylcytosine base pair mismatch, or the cytosine-hydroxymethylcytosine base pair mismatch is within a contiguous region of at least 10 nucleotides that are hybridized in the target/probe oligonucleotide. The methods also comprise contacting the target/probe oligonucleotide with Ag⁺, wherein Ag⁺ reversibly binds the cytosine-cytosine base pair mismatch, the cytosine-methylcytosine base pair mismatch, and the cytosine-hydroxymethylcytosine base pair mismatch in a differential manner thus increasing the hybridization stability of the target/probe oligonucleotide in a differential manner depending on the presence of a cytosine-cytosine base pair mismatch, the cytosine-methylcytosine base pair mismatch, and the cytosine-hydroxymethylcytosine base pair mismatch. The methods also comprise detecting the reversible binding of Ag⁺ to the mismatch. The amount of increase in the hybridization stability of the target/probe oligonucleotide discriminates whether the target ss-oligonucleotide or target strand of the ds-oligonucleotide contained a cytosine residue, a methylcytosine residue, or a hydroxymethylcytosine residue.

In certain embodiments of discriminating between a cytosine residue, a methylcytosine residue, and a hydroxymethylcytosine residue in a target single-stranded oligonucleotide (ss-oligonucleotide) or in a target strand of a double-stranded oligonucleotide (ds-oligonucleotide): at least 6, at least 7, at least 8, or at least 9 of the base-pairings within the contiguous hybridized region of at least 10 nucleotides are non-mismatched base-pairings; at least the target ss-oligonucleotide or target strand of the ds-oligonucleotide or probe molecule comprises an oligonucleotide from about 10, 12, 14, 16, or 19 to about 20, 25, 30, 40, 50, 60, 100 or more nucleotides in length; the probe molecule comprises a tag domain comprising a polydeoxycytosine covalently bound to the 3′-end, the 5′-end, or both the 3′-end and the 5′-end of the hybridizing region; the tag domain is poly(dC)₃₀; the method further comprising detecting the increase in the hybridization stability of the target/probe oligonucleotide; the increase in hybridization stability of the target/probe oligonucleotide is detected with a nanopore, PCR, gold nanoparticle, horseradish peroxidase, atomic force microscope, or immuo-PCR; and/or the increased hybridization stability of the ds-oligonucleotide is detected with a nanopore. In certain embodiments of discriminating between a cytosine residue, a methylcytosine residue, and a hydroxymethylcytosine residue in a target single-stranded oligonucleotide (ss-oligonucleotide) or in a target strand of a double-stranded oligonucleotide (ds-oligonucleotide) the increase is detected using a nanopore, and the nanopore detection of the increase in hybridization stability of the ds-oligonucleotide comprises: (a) applying a voltage to a sample containing the ds-oligonucleotide in a cis compartment of a duel chamber nanopore system, the voltage sufficient to drive translocation of the hybridized ds-oligonucleotide through a nanopore of said system by an unzipping process; and (b) analyzing an electrical current pattern in the nanopore system over time, wherein the increased hybridization stability of the ds-oligonucleotide in the presence of reversible Ag² binding produces an electrical current pattern that is different and distinguishable from an electrical current pattern produced by the ds-oligonucleotide in the absence of Ag⁺.

### BRIEF DESCRIPTION OF THE DRAWINGS

FIG. 1 illustrates the detection of a single T-Hg-T inter-strand lock (MercuLock) in the nanopore.

FIG. 2 shows discrimination of uracil and unmethylated cytosine with an inter-strand lock (MercuLock).

FIG. 3 shows site-specific detection of DNA methylation with an inter-strand lock (MercuLock).

FIG. 4 shows the detection of DNA containing different numbers and distribution of methylated cytosines.

FIG. 5 shows the sequences of targets and probes used to illustrate various embodiments: P_{T} (probe) is SEQ ID NO: 1; T_{T} (target) is SEQ ID NO: 2; T_{A} (target is SEQ ID NO: 3; T_{C} (target) is SEQ ID NO: 4; T_{rU} (target) is SEQ ID NO: 5; T_{U} (target) is SEQ ID NO: 6; T_{mC} (target) is SEQ ID NO: 7; Tₚ₁₆₋₁ (target, from p16 gene) is SEQ ID NO: 8; Tₚ₁₆₋₂ (target, from p16 gene) is SEQ ID NO: 9; Tp₁₆₋₃ (target, from p16 gene) is SEQ ID NO: 10; P_{C6} (probe) is SEQ ID NO: 11; P_{C8} (probe) is SEQ ID NO: 12; and P_{C14} (probe) is SEQ ID NO: 13; P_{C16} (probe) is SEQ ID NO: 14.

FIG. 6 shows lack of formation of an inter-strand lock with fully matched adenosine-thymine pair (A-T) and cytosine-thymine mismatch (C-T).

FIG. 7 shows Hg²⁺ concentration- and voltage-dependent frequency and duration of long blocks for the T_{T}·P_{T} hybrid.

FIG. 8 shows negative Ion Static Nanospray QTOF Mass Spectrum for dsDNA containing a T-T mismatched base pair in the presence of Hg²⁺.

FIG. 9 shows the location of tested CpG rich sequence in CDKN2A gene CpG island.

FIG. 10 shows current traces showing the translocation of the p16 gene fragment Tp16-1 and its bisulfite-converted sequence.

FIG. 11 shows the sequences of targets and probes used to illustrate various embodiments: 1C (SEQ ID NO: 15); 1mC (SEQ ID NO: 16); 1hmC (SEQ ID NO: 17); P1 (SEQ ID NO: 18); P2 (SEQ ID NO: 19).

FIG. 12 shows that Ag⁺ stabilizes DNA duplex containing C-C mismatches.

FIG. 13 shows interactions of Ag⁺ with DNA duplex containing mC-C and hmC-C mismatches.

FIG. 14 illustrates molecular dynamics simulations of DNA duplex containing C-C, mC-C and hmC-C mismatches.

FIG. 15 illustrates the nanopore recording platform.

FIG. 16 shows that ssDNA P1 interacts with the nanopore.

FIG. 17 shows melting temperature (Tm, ° C.) of the DNA C-C, mC-C and hmC-C with and without Ag⁺.

FIG. 18 shows that Ag⁺ doesn't interact with ssDNAs 1C, 1mC or 1hmC.

FIG. 19 shows that the addition of Ag⁺ decreased the residual current at different degrees for C-C and mC-C mismatches, but has no effect on hmC-C.

FIG. 20 shows that the DNA duplex C-C (ssDNA 1C hybridized with P1) interacts with the nanopore at 180 mV.

FIG. 21 shows MD simulation of a DNA duplex with the C-C mismatch that is coordinated with a Ag⁺.

FIG. 22 shows probability densities of hydrogen-bond lengths between N3 and O2 atoms of difference bases in a mismatched pair.

FIG. 23 shows the sequences of targets and probes used to illustrate various embodiments: BRAF_Sense (SEQ ID NO: 22); BRAF_V600E Sense (SEQ ID NO: 23); Probe_sense (SEQ ID NO: 24); Probe_sense 1 (1 mismatch at 5′ end) (SEQ ID NO: 25); Probe_sense 2 (1 mismatch next to the mutation site) (SEQ ID NO: 26); Probe_sense 3 (1 mismatch at the unzipping starting site) (SEQ ID NO: 27); BRAF_Anti-Sense (SEQ ID NO: 28); V600E_Anti-Sense (SEQ ID NO: 29); Probe_anti-sense (SEQ ID NO: 30); Probe_anti-sense_1 (2 mismatches at the unzipping starting site) (SEQ ID NO: 31); Probe_anti-sense_2 (2 mismatches before and after the mutation site) (SEQ ID NO: 32); Probe_anti-sense 3 (1 mismatch at the start+1 mismatch beside mutated site) (SEQ ID NO: 33).

FIG. 24 shows the BRAF-V600E mutant gene, anti-sense strand, and detection using Probe_anti-sense_1 in the absence of Hg²⁺.

FIG. 25 shows the BRAF-V600E mutant gene, anti-sense strand, and detection using Probe_anti-sense_1 in the presence of Hg²⁺.

FIG. 26 shows the BRAF-V600E mutant gene, anti-sense strand, and detection using Probe_anti-sense_2 in the absence of Hg²⁺.

FIG. 27 shows the BRAF-V600E mutant gene, anti-sense strand, and detection using Probe_anti-sense_2 in the presence of Hg²⁺.

### DETAILED DESCRIPTION

It is to be noted that the term “a” or “an” entity refers to one or more of that entity; for example, “a probe molecule” is understood to represent one or more probe molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation and nucleic acid sequences are written from their 5′- to 3′-end.

For convenience, certain definitions of terms as used in this disclosure are listed together below. Definitions, however, are not limited to this section and the definition of certain other terms may be provided for elsewhere.

As used herein, a base pair or base pairing refers to Watson-Crick base pairs, i.e., A-T, U-T, and C-G. Base pairing can occur between two strands of separate nucleic acid molecules or between two single stranded regions of the same nucleic acid molecule. Base pairing can occur between DNA-DNA base pair residues, RNA-RNA base pair residues, and DNA-RNA base pair residues. As is well known in the art, “hybridization” of nucleic acid molecules occurs in regions where base pairing occurs. Base pairing mismatches (e.g.: T-T, U-T, C-C, A-A, A-G, etc.) however, can reside within regions of hybridization.

As used herein, the term “oligonucleotide” refers to a polymeric nucleic acid molecule that can be either single-stranded or double-stranded. In certain embodiments, an oligonucleotide is from about 8 to about 24 nucleotides in length, for example, 8, 9, 10, 11, 12, 13, 14, 15, 16, 17, 18, 19, 20, 21, 22, 23 or 24 nucleotides in length. In certain embodiments, an oligonucleotide is up to about 25 nucleotides, up to about 30 nucleotides, up to about 40 nucleotides, up to about 50 nucleotides, or up to about 60 nucleotides in length, or up to about 100 nucleotides in length. In certain embodiments, an oligonucleotide may be more than 100 nucleotides in length. In certain embodiments, an oligonucleotide may be between 8 and 100 nucleotides in length. In certain embodiments, an oligonucleotide may be between 8 and 1000 nucleotides in length.

As used herein, the term “inter-strand lock” refers to a nucleotide base pairing associated with an ion, wherein the association is a reversible binding that increases the stability of the base pairing and can increase the stability of the double-stranded oligonucleotide comprising the base pairing. The base pairing can be a Watson-Crick mismatched base pairing, for example, but not limited to: T-T, U-T, and C-C. In certain embodiments, the ion is a mercuric ion (Hg²⁺) or a silver ion (Ag⁺). Inter-strand locks are designated herein by specifying the base pairs and ion, such as T-Hg-T or C-Ag-C.

As used herein, the term “MercuLock” refers to a specific T-Hg-T, rU-Hg-T, or U-Hg-T inter-strand lock.

As used herein, the term “double-stranded” when used in reference to a nucleic acid molecule refers to a nucleic acid molecule that is at least partially double-stranded, meaning that the nucleic acid molecule could comprise regions that are both single-stranded and double-stranded, unless it is otherwise stated that the entire length of the nucleic acid molecule is double-stranded. For example, in certain embodiments, a double-stranded nucleic acid has a double stranded region of at least 10 contiguous base pairs.

As used herein, reference to a “first single-stranded oligonucleotide” and a “second single stranded oligonucleotide” to form the ds-oligonucleotide means two separate ss-oligonucleotides hybridized to form the ds-oligonucleotide, and unless otherwise specified, does not include a single oligonucleotide hybridizing on itself, such as for example through hairpin structure.

As used herein a probe molecule or other oligonucleotide may be chosen or designed to form a base pair mismatch with a particular residue on another oligonucleotide, such as a target oligonucleotide.

A versatile detection method has been discovered that utilizes a base-pair-specific inter-strand lock for genetic and epigenetic detection. Reagents, devices, etc., for implementing the method have also been discovered and/or developed. In certain embodiments, compounds have been identified to be able to specifically bind certain mismatched base pairs. Such binding can strengthen the base-pair hybridization in orders of magnitude, forming a so-called reversible inter-strand lock that can greatly stabilize double-stranded nucleic acid fragments. In certain embodiments, it is contemplated that in genetic and epigenetic detections, special probes can be designed such that when hybridized with a target sequence, the probe-target hybrid can form an inter-strand lock at a specific base: for example a site for driver mutation, CpG methylation, or gene damage. The inter-strand lock can be detected, for example, by detecting an increase in hybridization stability by various known methods. In certain embodiments, a nanopore single-molecule sensor can be used to sensitively detect the inter-strand lock in a gene at the single-molecule and single base-pair levels.

Certain aspects are based on a single-molecule and single-base investigation of a base-pair specific metal ion/nucleic acids interaction. One discovery is a base-pair specific metal ion-nucleic acid interaction, and in particular, it has been discovered that a uracil-thymine mismatch at a CpG site can be bound with a divalent mercuric ion (Hg²⁺). The metal binding creates a reversible inter-strand lock that enhances the hybridization strength. In certain embodiments, the hybridization strength is increased by nearly two orders of magnitude. In contrast, the 5-methyl cytosine-thymine mismatch does not form such a tight association with Hg²⁺ and the thus the presence of Hg²⁺ does not increase the hybridization strength to the same degree. Thus uracil and methylated cytosine can be discriminated. In certain embodiments, uracil and methylated cytosine can be discriminated by their signatures in a nanopore. Further, because uracil is converted from unmethylated cytosine by the bisulfite treatment, the identity of uracil corresponds to an unmethylated cytosine. Therefore, in certain embodiments, methods are provided wherein the presence of a cytosine in an oligonucleotide (which can be converted to uracil by bisulfite treatment) or the presence of a methylated cytosine in an oligonucleotide (which is not converted to uracil by bisulfite treatment) can be determined. In certain embodiments, methods are provided wherein methylated and unmethylated cytosine in an oligonucleotide can be discriminated or distinguished.

In another aspect, a cytosine-cytosine (C-C) mismatch can be bound with a silver ion (Ag⁺) to form an inter-strand lock (C-Ag-C). In certain embodiments, if a cytosine is 5′-methylcytosine or 5′-hydroxymethylcytosine, the stability of the inter-strand lock will be changed. This difference in stability can be detected. In certain embodiments, the difference in stability is detected using a nanopore single-molecule sensor. For example, the DNA duplex containing single cytosine-cytosine (C-C), cytosine-methylcytosine (C-mC) and cytosine-hydroxymethylcytosine (C-hmC) mismatches can be discriminated by their interactions with Ag⁺ inside an alpha-hemolysin nanopore. Molecular dynamics simulations revealed that the paring of a C-C mismatch through hydrogen bond results in a binding site for cations, such as K⁺ and Ag⁺. Cytosine modifications such as mC and hmC disrupted both the hydrogen bonds, which subsequently disrupts Ag⁺ binding. As a result, these modifications can be distinguished by differences in the stability of DNA-Ag⁺ complexes. As a result, in certain embodiments these modifications can be distinguished by nanopore detection of differences in the stability of DNA-Ag⁺ complexes.

In another aspect, because a thymine-thymine (T-T) mismatch can be bound with a divalent mercuric ion (Hg²⁺) to form a strong inter-strand lock (T-Hg-T) it is contemplated that for any driver mutation or gene damage that involves a thymine, a probe can be designed to examine if a thymine-thymine inter-strand lock can be formed, therefore determining whether the mutation or damage occurrence.

Another aspect is drawn to microRNA detection wherein a probe can be designed to form inter-strand lock with the target microRNA, based on the inter-strand lock formations described herein, to enhance the target microRNA/probe hybridization. This has two functions: a) The formation of one or more inter-strand locks increase the microRNA:probe hybrid amount, enhancing the PCR sensitivity; and b) forming inter-strand lock at specific site allows discriminating sequence-similar microRNAs with high specificity.

Another aspect is drawn to the construction of inter-strand locks when using an anti-sense fragment to bind the target gene which enhances the bind affinity and specificity, thus enhancing the gene regulation efficiency and improve therapy. Another aspect is drawn to the construction of inter-strand locks at designed positions that can enhance the stability of DNA or RNA nanostructures such as origami.

Inter-strand locks can be detected by numerous widely known methods such as PCR and qRT-PCR approaches and approaches that involve signal amplification including, but not limited to: a nanoparticle such as gold nanoparticle, horseradish peroxidase, atomic force microscope, and immuo-PCR.

The disclosed inter-strand lock method can be combined with a nanoparticle platform such as gold nanoparticle (AuNP). AuNP has two basic properties for nucleic acid detection: 1) AuNP can assemble or aggregate by the target nucleic acids fragment. The aggregated AuNP change color from red to purple, allowing visually identify the target. 2) Aggregated AgNP features a sharp color change along with the temperature increase. This allows extreme sensitive melting temperature measurement. Since the inter-strand lock on dsDNA can increase the hybridization strength, AuNP can be used to detect it.

The disclosed inter-strand lock method can also be combined with a PCR platform. The inter-strand lock enhances the hybridization between the template and the primer, thus resulting in higher annealing temperatures.

The inter-strand lock method can be combined with an atom force microscope platform. The inter-strand lock enhances the hybridization, which can reveal the force profile for specific target detection, such as detect multiple methylation sites along the nucleic acids sequence.

The inter-strand lock method can be combined with a horseradish peroxidase method. The inter-strand lock enhances the binding of the probe with the target sequence fragment, then horseradish peroxidase attached to the probe can amplify the signal.

The inter-strand lock method can be used to detect single nucleotide polymorphisms or driver mutation in disease detection, and gene damage, and any mismatch. The detection targets can be both DNAs and RNAs.

Further, the inter-strand lock method can be used to assemble nucleic acid nanostructures such as origami.

Certain embodiments utilize a robust nanopore sensing system that enables sensitive, selective and direct detection, differentiation and quantification of nucleic acid interactions, such as the hybridization stability of double-stranded oligonucleotides. Detailed disclosure of such nanopore sensing systems and methods of their utilization are described in U.S. application Ser. No. 13/810,105, which is expressly incorporated by reference herein in its entirety. To the extent that there are any inconsistencies between disclosures, this disclosure is controlling.

In certain embodiments, nanopore sensing technology can be employed to detect an increase in hybridization stability in a double-stranded nucleic acid molecule such as a double-stranded oligonucleotide, as for example, an increase in hybridization stability resulting from an inter-strand lock formed at the site of certain base pair mismatches. As described herein, inter-strand locks at certain base pair mismatches may form when the mismatched residues are reversibly bound by a mercuric ion (Hg²⁺) or silver ion (Ag⁺). Furthermore, the disclosed technology has the potential for non-invasive and cost-effective early diagnosis and continuous monitoring of cancer markers.

A representative nanopore sensing systems includes 1) a nanopore allowing translocation of a single-stranded oligonucleotide, 2) a power source providing a pre-determined voltage as driving force to induce unzipping of a double-stranded oligonucleotide, 3) a molecule to be examined, such as one comprising a double-stranded oligonucleotide, which is loaded into the nanopore and which in the pore produces certain identifiable current signal changes, and 4) a method/device for detecting current changes. The sensing chamber of a representative nanopore sensing system includes a cis compartment, and a trans compartment, which are divided by a partition. Both compartments are filled with a pre-selected recording solution, as an example, 1 M KCl. The partition has an opening in its center region, over which a lipid bilayer is formed, and the nanopore is plugged through the lipid bilayer. The power source provides a voltage that is loaded through a pair of electrodes in the two compartments; the current detector, such as a pico-Ampere amplifier is connected to monitor the current changes. Upon the testing, a mixture sample of the molecule to be examined is loaded into the cis compartment.

A representative nanopore has a conical or funnel shape with two openings, the cis opening at the wide end and the trans opening, down the narrow end. During detection the molecule to be examined is captured into the nanocavity. The voltage then drives the molecule. For example, the voltage drives a double-stranded oligonucleotide to unzip at the constriction, with a portion first traversing through the β-barrel and out of the trans opening, which then may be followed by the traversal of other portions.

The nanopore may be any ion channel of cone-shape or any asymmetrical shape with a wide and a narrow opening plugged into the planar lipid bilayer that has a wider cavity followed by a narrow channel that can facilitate unzipping translocation events. The nanopore may be any existing protein ion channels, such as the α-hemolysin transmembrane protein pore adopted in the examples disclosed herein, or various synthetic pores fabricated using fashion nanotechnologies with abiotic materials such as silicon.

In certain representative methods, a nanopore is used to detect the hybridization stability of a ds-oligonucleotide, such as an increase in hybridization stability resulting from the formation of an inter-strand lock formed by certain base pair mismatches and Hg²⁺ or Ag⁺. Such methods comprises applying a voltage to a sample containing the ds-oligonucleotide in a cis compartment of a duel chamber nanopore system, wherein the voltage is sufficient to drive translocation of the hybridized ds-oligonucleotide through a nanopore of the system by an unzipping process and analyzing an electrical current pattern in the nanopore system over time. The increase in hybridization stability of the ds-oligonucleotide can be detected at least because its hybridization stability in the presence of Hg²⁺ or Ag⁺ produces an electrical current pattern that is different and distinguishable from an electrical current pattern produced by the same ds-oligonucleotide structure in the absence of Hg²⁺ or Ag⁺, respectively. The increase in hybridization stability of the ds-oligonucleotide due to a base pair mismatch may be detected because its hybridization stability in the presence of Hg²⁺ or Ag⁺ produces an electrical current pattern that is different and distinguishable from an electrical current pattern produced by a ds-oligonucleotide structure with a different base pairing at the site of the inter-strand lock, even in the presence of Hg²⁺ or Ag⁺.

In certain embodiments, whether for use with Hg²⁺ inter-strand locks or Ag⁺ inter-strand locks, one or more oligonucleotides comprises a tag domain, for example as described in U.S. application Ser. No. 13/810,105, which is expressly incorporated by reference herein in its entirety. To the extent that there are any inconsistencies between disclosures, this disclosure is controlling. In a nanopore system, such tag domains can allow one to discriminate double-stranded nucleic acid molecule unzipping events from noise. Thus, in certain embodiments, including the use of a nanopore for detection, a tag domain aids in the detection of an increase in hybridization stability of a ds-oligonucleotide. The tag domain may be placed either at the 3-end, the 5′-end, or at both the 3′-end and 5′-end of a hybridization region or target sequence. In certain embodiments, the tag domain is covalently bound to the oligonucleotide. The tag domain may be attached directly adjacent to or at a distance from the hybridization region or target sequence, such as separated by a linker sequence. Target sequences include, but are not limited to, sequences containing a residue to form a mismatch for increasing the hybridization stability of a ds-oligonucleotide as described elsewhere herein or a sequence including a cytosine residue for determining whether the cytosine residue is modified or un-modified as described elsewhere herein. In certain embodiments, a target sequence may part of a probe molecule. Therefore, in certain embodiments, a probe molecule comprises a tag domain. The tag domain can comprise a charged polymer of any length, for example a charged polypeptide or a charged oligonucleotide. In certain embodiments, the tag domain may be of any charged single chain molecule with sufficient length to assist the unzipping translocation through a nanopore driven by voltage. In certain embodiments, a charged polypeptide comprises at least two positively charged amino acid residues and/or at least two aromatic amino acid residues.

In certain embodiments, the tag domain is an oligonucleotide such as a negatively charged single-stranded nucleic acid. In certain embodiments, the tag domain is an oligonucleotide that does not hybridize during the increase in hybridization stability, the detection of such an increase, or the discrimination of certain residues as described elsewhere herein. Advantages of such nucleic acid tag domains include, but are not limited to, extremely low cost of synthesis and controllable charge by pH, salt concentration and temperature. Such nucleic acid tag domains can comprise homopolymers, heteropolymers, copolymers or combinations thereof. In certain embodiments, the lengths of such nucleic acid terminal extensions can range from about 1 or 2 nucleotides to about 50 nucleotides. In still other embodiments, the nucleic acid extensions can range in length from about 5 to about 40 nucleotides, about 15 to about 35 nucleotides, or from about 20 to about 35 nucleotides.

The tag domain may be an oligonucleotide such as poly(dC)ₙ, poly(dA)ₙ, and or poly(dT)ₙ. For example, when α-hemolysin transmembrane protein pore is employed as the nanopore, the poly(dC) tag is more preferred over poly(dA) or poly(dT) tags; furthermore, the poly(dC)₃₀ is much more efficient in generating signature events than that with a shorter tag such as poly(dC)₈. The capture rate can be further enhanced once combined with other effective approaches, including detection at high voltage, use of engineered pores with designed charge profile in the lumen, and detection in asymmetrical salt concentrations between both sides of the pore.

An representative tag domain provided herewith is homopolymer poly(dC)₃₀. However, a heteropolymeric sequence, including but not limited to, di- or tri-nucleotide heteropolymers such as CTCTCTCT . . . , or CATCATCAT . . . , can also be used. In certain embodiments, co-polymers comprising abases or polyethylene glycol (PEG) can be used in the tag domain. These co-polymers, or domains thereof in a terminal extension, can confer new functions on the tag domain. An abase is a nucleotide without the base, but carries a negative charge provided by the phosphate. As the dimension of abase is narrower than normal nucleotides, it may generate a signature event signal different from that formed by the neighbor nucleotides. PEG is not charged. Without seeking to be limited by theory, it is believed that when the PEG domain in a nucleic acid sequence is trapped in the pore, it can reduce the driving force, thus precisely regulating the dissociation of the probe/target complex. Therefore, PEG (or other polyglycols) may be used, in particular, as a tag domain to facility multiplexing. For example, different tag domains may be utilized simultaneously within one nanopore system to provide for differential determinations as described in U.S. patent application Ser. No. 14/213,140, which is expressly incorporated by reference herein in its entirety. To the extent that there are any inconsistencies between disclosures, this disclosure is controlling.

Certain embodiments are drawn to methods of increasing the hybridization stability of a double-stranded oligonucleotide comprising a thymine-thymine (T-T) or a uracil-thymine (U-T) base pair mismatch. It is understood that whereas DNA generally comprises thymine and RNA comprises uracil, uracil can also occur in DNA. Except as otherwise specifically distinguished herein, a U-T base pair mismatch can comprise either the ribo- or deoxyribo-forms of uracil. In certain embodiments, the T-T or U-T base pair mismatch occurs in a hybridized region of the ds-oligonucleotide. It has been discovered that an increase in hybridization stability between the two strands of a ds-oligonucleotide can be achieved by the reversible binding of Hg²⁺ to the T-T or U-T base pair mismatch. As referred to herein, this increase in hybridization stability that is formed between the two strands of a ds-oligonucleotide by the reversible binding of Hg²⁺ (or as described elsewhere herein, Ag⁺) to a specific pair mismatch is an inter-strand lock (also referred to as MercuLock when used to describe T-Hg-T or U-Hg-T). Therefore, certain embodiments comprise reversibly binding Hg²⁺ to the mismatch. This increase in hybridization stability can be determined, for example, in comparison to the hybridization stability of the molecule in the absence of Hg²⁺. This increase in hybridization stability can be determined by a number of different detection methods including, but not limited to, measuring the melting temperature, various optical measurements which distinguish between single- and double-stranded nucleic acids, various techniques based on the polymerase chain reaction such as qRT-PCR, nanopore detection, and various other electrical detection methods. In certain embodiments, the increase in hybridization stability is detected using a nanopore or by using qRT-PCR. In certain embodiments, the increase in hybridization stability is detected using a nanopore according to methods described elsewhere herein. Although the methods of determining an increase in the hybridization stability of a double-stranded oligonucleotide (ds-oligonucleotide) comprising a thymine-thymine (T-T) or a uracil-thymine (U-T) base pair mismatch may include detection using a nanopore or qRT-PCR, such methods are in no way meant to be limited to these detection methods.

In certain embodiments, the T-T or U-T base pair mismatch is within a hybridized region of the ds-oligonucleotide of at least 10 contiguous nucleotides. Although multiple base pair mismatches may reside within a hybridized region, in certain embodiments, at least 6, at least 7, at least 8, or at least 9 of the base-pairings within a contiguous hybridized region of at least 10 nucleotides are non-mismatched base-pairings. In certain embodiments, the hybridized region is a contiguous region of at least 11, at least 12, at least 13, at least 14, at least 15, at least 16, at least 17, at least 18, or at least 19 nucleotides. In certain embodiments, the hybridized region is a contiguous region of up to about 20 nucleotides, about 30 nucleotides, about 40 nucleotides, or about 50 nucleotides. In certain embodiments, the hybridized region is a contiguous region of more than 50 nucleotides. In certain embodiments, the hybridized region is a contiguous region of between about 10, 12, 14, or 16 to about 20, 25, 30, 40, 50, 60, 100, or more nucleotides. In certain embodiments, the hybridized region is a contiguous region of between about 20, 25, 30, 40, or 50 to about 60, 80, 100, or more nucleotides.

In certain embodiments, the ds-oligonucleotide is formed from two single-stranded oligonucleotides before or while the hybridization stability of the double-stranded oligonucleotide is increased. Such methods comprise hybridizing a first single-stranded oligonucleotide to a second single stranded oligonucleotide to form the ds-oligonucleotide comprising the T-T or U-T base pair mismatch. That is, in certain embodiments, the hybridized region is not formed by a single nucleic acid molecule self-hybridizing. In certain embodiments, one or both of the first ss-oligonucleotide and the second ss-oligonucleotide comprise an oligonucleotide of at least 10, at least 11, at least 12, at least 13, at least 14, at least 15, or at least 16 nucleotides in length. In certain embodiments, one or both of the ss-oligonucleotide and the second ss-oligonucleotide may be up to about 20 nucleotides in length, about 30 nucleotides in length, about 40 nucleotides in length, about 50 nucleotides in length, or about 60 nucleotides in length. In certain embodiments, one or both of the ss-oligonucleotides may be more than 60 nucleotides in length. In certain embodiments, one or both of the ss-oligonucleotide and the second ss-oligonucleotide may be from about 10, 12, 14, 16, or 19 to about 20, 25, 30, 40, 50, 60, 100 or more nucleotides in length. In certain embodiments, one or both of the ss-oligonucleotide and the ss-oligonucleotide may be from about 20, 30, 40, or 50 to about 60, 80, 100, or more nucleotides in length.

Once formed, the ds-oligonucleotide containing the T-T or U-T mismatch is contacted with Hg²⁺. It is understood that a source Hg²⁺ could be added at any point, for example before the two ss-oligonucleotides hybridize or after they have hybridized, as long as Hg²⁺ is contacted with the ds-oligonucleotide containing the T-T or U-T mismatch. In certain embodiments, Hg²⁺ is provided by the addition of HgCl₂.

In certain embodiments, the base pair mismatch is a T-T mismatch. In certain embodiments, the mismatch is a rU-T mismatch. In certain embodiments, the base pair mismatch is a U-T mismatch.

Certain embodiments are drawn to methods of detecting a thymine-thymine (T-T) base pair mismatch or a uracil-thymine (U-T) base pair mismatch in a double-stranded oligonucleotide (ds-oligonucleotide). The methods comprise reversibly binding Hg²⁺ to the T-T or U-T base pair mismatch. It has been discovered that Hg²⁺ binding to T-T or U-T base pair mismatch increases the hybridization stability of the ds-oligonucleotide. The increase in hybridization stability can be determined, for example, in comparison to hybridization stability in the absence of Hg²⁺ reversible binding. This increase in hybridization stability can be determined by a number of different detection methods including, but not limited to, measuring the melting temperature, various optical measurements which distinguish between single- and double-stranded nucleic acids, various techniques based on the polymerase chain reaction such as qRT-PCR, nanopore detection, and various other electrical detection methods. Detection of increased hybridization stability of the ds-oligonucleotide in the presence of Hg²⁺ is indicative of a T-T or U-T base pair mismatch.

In certain embodiments, the increase in hybridization stability is detected using a nanopore or by using qRT-PCR. In certain embodiments, the increase in hybridization stability is detected using a nanopore. Although the methods of detecting a thymine-thymine (T-T) base pair mismatch or a uracil-thymine (U-T) base pair mismatch in a double-stranded oligonucleotide (ds-oligonucleotide) may include detection using a nanopore or qRT-PCR, such methods are in no way meant to be limited to these detection methods.

In certain embodiments, detection of the increase in hybridization stability of the ds-oligonucleotide using a nanopore comprises applying a voltage to a sample containing the ds-oligonucleotide in a cis compartment of a duel chamber nanopore system wherein the voltage is sufficient to drive translocation of the hybridized ds-oligonucleotide through a nanopore of said system by an unzipping process and analyzing an electrical current pattern in the nanopore system over time, wherein the increased hybridization stability of the ds-oligonucleotide in the presence of reversible Hg²⁺ binding produces an electrical current pattern that is different and distinguishable from an electrical current pattern produced by the ds-oligonucleotide in the absence of Hg²⁺. The presence of reversible Hg²⁺ binding to the mismatch may also produce an electrical current pattern that is different and distinguishable from an electrical current pattern produced by a ds-oligonucleotide with a different base pairing at the inter-strand lock site.

In certain embodiments, the T-T or U-T base pair mismatch is within a hybridized region of at least 10 contiguous nucleotides. Although multiple base pair mismatches may reside within a hybridized region, in certain embodiments, at least 6, at least 7, at least 8, or at least 9 of the base-pairings within a contiguous hybridized region of at least 10 nucleotides are non-mismatched base-pairings. In certain embodiments, the hybridized region is a contiguous region of at least 11, at least 12, at least 13, at least 14, at least 15, at least 16, at least 17, at least 18, or at least 19 nucleotides. In certain embodiments, the hybridized region is a contiguous region of up to about 20 nucleotides, about 30 nucleotides, about 40 nucleotides, or about 50 nucleotides. In certain embodiments, the hybridized region is a contiguous region of more than 50 nucleotides. In certain embodiments, the hybridized region is a contiguous region of more than 50 nucleotides. In certain embodiments, the hybridized region is a contiguous region of between about 10, 12, 14, or 16 to about 20, 25, 30, 40, 50, 60, 100, or more nucleotides. In certain embodiments, the hybridized region is a contiguous region of between about 20, 25, 30, 40, or 50 to about 60, 80, 100, or more nucleotides.

In certain embodiments, the ds-oligonucleotide is formed from two single-stranded oligonucleotides before or while the hybridization stability of the double-stranded oligonucleotide is increased. Such methods comprise hybridizing a first single-stranded oligonucleotide to a second single stranded oligonucleotide to form the ds-oligonucleotide comprising the T-T or U-T base pair mismatch. In certain embodiments, one or both the first ss-oligonucleotide and the second ss-oligonucleotide comprise an oligonucleotide of at least 10, at least 11, at least 12, at least 13, at least 14, at least 15, or at least 16 nucleotides in length. In certain embodiments, one or both of the ss-oligonucleotide and the second ss-oligonucleotide may be up to about 20 nucleotides in length, about 30 nucleotides in length, about 40 nucleotides in length, about 50 nucleotides in length, or about 60 nucleotides in length. In certain embodiments, one or both of the ss-oligonucleotide and the second ss-oligonucleotide may be from about 10, 12, 14, 16, or 19 to about 20, 25, 30, 40, 50, 60, 100 or more nucleotides in length. In certain embodiments, one or both of the ss-oligonucleotide and the ss-oligonucleotide may be of from about 20, 30, 40, or 50 to about 60, 80, 100, or more nucleotides in length.

Once formed, the ds-oligonucleotide containing the T-T or U-T mismatch is contacted with Hg²⁺. It is understood that a source Hg²⁺ could be added at any point, for example before the two ss-oligonucleotides hybridize or after they have hybridized, as long as Hg²⁺ is contacted with the ds-oligonucleotide containing the T-T or U-T mismatch. In certain embodiments, Hg²⁺ is provided by the addition of HgCl₂.

In certain embodiments, the base pair mismatch is a T-T mismatch. In certain embodiments, the mismatch is a rU-T mismatch. In certain embodiments, the base pair mismatch is a U-T mismatch.

Although it may be known that a certain nucleic acid molecule (for example a target oligonucleotide) comprises one or more cytosine residues, it may be useful to further determine whether those residues are methylated or un-methylated. Thus, certain embodiments are drawn to methods of determining whether a cytosine residue in a target single-stranded oligonucleotide (ss-oligonucleotide) or in a target strand of a double-stranded oligonucleotide (ds-oligonucleotide) is a methylated cytosine residue or an un-methylated cytosine residue. It is known that bisulfite treatment of a nucleic acid molecule can convert cytosine residues to uracil. However, this treatment usually does not convert methylated cytosine, such as 5′-methylcytosine, to uracil.

In certain embodiments, a target ss-oligonucleotide or target strand of the ds-oligonucleotide is treated with bisulfite to convert an un-methylated cytosine residue to a uracil residue but wherein said treatment does not convert a methylated cytosine residue to a uracil residue. It will be apparent that if an un-methylated cytosine residue is not present in the target oligonucleotide (and/or not present at the residue of interest), it will not be converted to uracil and vice versa. After bisulfite treatment, the target ss-oligonucleotide or target strand of the ds-oligonucleotide is hybridized with a probe molecule. In certain embodiments, the probe molecule is designed to form a U-T mismatch if a uracil is present at the residue to be investigated. This hybridization forms an at least partially double-stranded target/probe oligonucleotide that comprises a thymine residue base pair mismatched with the converted uracil residue (U-T), if present. Alternatively, this hybridization forms an at least partially double-stranded target/probe complex that comprises a thymine residue base pair mismatched with the un-converted methylated cytosine residue (mC-T), if present.

In certain embodiments, the U-T base pair mismatch is within a hybridized region of at least 10 contiguous nucleotides. Although multiple base pair mismatches may reside within a hybridized region, in certain embodiments, at least 6, at least 7, at least 8, or at least 9 of the base-pairings within a contiguous hybridized region of at least 10 nucleotides are non-mismatched base-pairings. In certain embodiments, the hybridized region is a contiguous region of at least 11, at least 12, at least 13, at least 14, at least 15, at least 16, at least 17, at least 18, or at least 19 nucleotides. In certain embodiments, the hybridized region is a contiguous region of up to about 20 nucleotides, about 30 nucleotides, about 40 nucleotides, or about 50 nucleotides. In certain embodiments, the hybridized region is a contiguous region of more than 50 nucleotides. In certain embodiments, the hybridized region is a contiguous region of more than 50 nucleotides. In certain embodiments, the hybridized region is a contiguous region of between about 10, 12, 14, or 16 to about 20, 25, 30, 40, 50, 60, 100, or more nucleotides. In certain embodiments, the hybridized region is a contiguous region of between about 20, 25, 30, 40, or 50 to about 60, 80, 100, or more nucleotides.

The hybridized target/probe oligonucleotide is contacted with Hg²⁺. It has been discovered that wherein Hg²⁺ reversibly binds the U-T base pair mismatch it does not bind the mC-T mismatch. Although it may be understood that the mC-T mismatch may not absolutely be devoid of any reversible binding with Hg²⁺, the magnitude of difference between the reversible binding of Hg²⁺ with the U-T base pair mismatch and the mC-T base pair mismatch is distinguishable and as such, for the purposes of this disclosure, any amount of Hg²⁺ reversible binding that occurs with the mC-T mismatch is considered to be an absence reversible Hg²⁺ binding. Thus, the presence or absence of the reversible binding of Hg²⁺ is detected wherein the presence indicates that the cytosine residue in the target ss-oligonucleotide or in the target strand of the ds-oligonucleotide was un-methylated and the absence indicates that the cytosine residue in the target ss-oligonucleotide or in the target strand of the ds-oligonucleotide was methylated.

As described elsewhere herein, reversible Hg²⁺ binding to a U-T base pair mismatch can increase the hybridization stability of a double-stranded nucleic acid molecule. This increase in hybridization stability can be determined, for example, in comparison to the hybridization stability of the molecule in the absence of Hg²⁺, by a number of different detection methods. This increase in hybridization stability can be determined by a number of different detection methods including, but not limited to, measuring the melting temperature, various optical measurements which distinguish between single- and double-stranded nucleic acids, various techniques based on the polymerase chain reaction such as qRT-PCR, nanopore detection, and various other electrical detection methods. In certain embodiments, the increase in hybridization stability is detected using a nanopore or by using qRT-PCR. In certain embodiments, the increase in hybridization stability is detected using a nanopore according to method described elsewhere herein. Although the methods of determining whether a cytosine residue in a target single-stranded oligonucleotide (ss-oligonucleotide) or in a target strand of a double-stranded oligonucleotide (ds-oligonucleotide) is a methylated cytosine residue or an un-methylated cytosine residue may include detection using a nanopore or qRT-PCR, such methods are in no way meant to be limited to these detection methods.

In certain embodiments, at least one of the target ss-oligonucleotide or target strand of the ds-oligonucleotide and the probe molecule comprise an oligonucleotide of at least 10, at least 11, at least 12, at least 13, at least 14, at least 15, or at least 16 nucleotides in length. In certain embodiments, at least one may be up to about 20 nucleotides in length, about 30 nucleotides in length, about 40 nucleotides in length, about 50 nucleotides in length, or about 60 nucleotides in length. In certain embodiments, at least one may be more than 60 nucleotides in length. In certain embodiments, at least one may be from about 10, 12, 14, 16, or 19 to about 20, 25, 30, 40, 50, 60, 100 or more nucleotides in length. In certain embodiments, at least one may be from about 20, 30, 40, or 50 to about 60, 80, 100, or more nucleotides in length.

Once formed, the ds-oligonucleotide containing the U-T mismatch is contacted with Hg²⁺. It is understood that a source Hg²⁺ could be added at any point, for example before the two ss-oligonucleotides hybridize or after they have hybridized, as long as Hg²⁺ is contacted with the ds-oligonucleotide containing U-T mismatch. In certain embodiments, Hg²⁺ is provided by the addition of HgCl₂.

In certain embodiments, the target ss-oligonucleotide or target strand of the ds-oligonucleotide comprises a plurality of cytosine residues which may or may not be methylated. Therefore, certain embodiments herein are drawn to methods of determining whether one or more of such cytosine residues are methylated or un-methylated. In certain embodiments, multiple probe molecules are utilized that hybridize with the target oligonucleotide. The probe molecules are able to differentiate the different cytosine residues by forming various base pair mismatches, thus allowing the determination at multiple potential methylation sites. In certain embodiments, different probe molecules may comprise tag domains that allow their differentiation and therefore all for multiplex discrimination.

Certain embodiments are drawn to methods of increasing the hybridization stability of a double-stranded oligonucleotide (ds-oligonucleotide) comprising a cytosine-cytosine (C-C) or a methylated cytosine-cytosine (mC-C) base pair mismatch. In certain embodiments, the C-C or mC-C base pair mismatch occurs in a hybridized region of the ds-oligonucleotide. It has been discovered that an increase in hybridization stability between the two strands of a ds-oligonucleotide can be achieved by the reversible binding of Ag⁺ to the C-C base pair mismatch and to a lesser degree to the mC-C base pair mismatch. As referred to herein, this increase in hybridization stability that is formed between the two strands of a ds-oligonucleotide by the reversible binding of Ag⁺ (or as described elsewhere herein, Hg²⁺) to a specific pair mismatch is an inter-strand lock. Therefore, certain embodiments comprise reversibly binding Ag⁺ to the mismatch. This increase in hybridization stability can be determined, for example, in comparison to the hybridization stability of the molecule in the absence of Ag⁺, by a number of different detection methods. This increase in hybridization stability can be determined by a number of different detection methods including, but not limited to, measuring the melting temperature, various optical measurements which distinguish between single- and double-stranded nucleic acids, various techniques based on the polymerase chain reaction such as qRT-PCR, nanopore detection, and various other electrical detection methods. In certain embodiments, the increase in hybridization stability is detected using a nanopore or by using qRT-PCR. In certain embodiments, the increase in hybridization stability is detected using a nanopore according to method described elsewhere herein. Although the methods of determining an increase in the hybridization stability of a double-stranded oligonucleotide (ds-oligonucleotide) comprising a C-C or a mC-C base pair mismatch may include detection using a nanopore or qRT-PCR, such methods are in no way meant to be limited to these detection methods.

In certain embodiments, the C-C or mC-C base pair mismatch is within a hybridized region of at least 10 contiguous nucleotides. Although multiple base pair mismatches may reside within a hybridized region, in certain embodiments, at least 6, at least 7, at least 8, or at least 9 of the base-pairings within a contiguous hybridized region of at least 10 nucleotides are non-mismatched base-pairings. In certain embodiments, the hybridized region is a contiguous region of at least 11, at least 12, at least 13, at least 14, at least 15, at least 16, at least 17, at least 18, or at least 19 nucleotides. In certain embodiments, the hybridized region is a contiguous region of up to about 20 nucleotides, about 30 nucleotides, about 40 nucleotides, or about 50 nucleotides. In certain embodiments, the hybridized region is a contiguous region of more than 50 nucleotides. In certain embodiments, the hybridized region is a contiguous region of more than 50 nucleotides. In certain embodiments, the hybridized region is a contiguous region of between about 10, 12, 14, or 16 to about 20, 25, 30, 40, 50, 60, 100, or more nucleotides. In certain embodiments, the hybridized region is a contiguous region of between about 20, 25, 30, 40, or 50 to about 60, 80, 100, or more nucleotides.

In certain embodiments, the ds-oligonucleotide is formed from two single-stranded oligonucleotides before or while the hybridization stability of the double-stranded oligonucleotide is increased. Such methods comprise hybridizing a first single-stranded oligonucleotide to a second single stranded oligonucleotide to form the ds-oligonucleotide comprising the C-C or mC-C base pair mismatch. In certain embodiments, one or both the first ss-oligonucleotide and the second ss-oligonucleotide comprise an oligonucleotide of at least 10, at least 11, at least 12, at least 13, at least 14, at least 15, or at least 16 nucleotides in length. In certain embodiments, one or both of the ss-oligonucleotide and the second ss-oligonucleotide may be up to about 20 nucleotides in length, about 30 nucleotides in length, about 40 nucleotides in length, about 50 nucleotides in length, or about 60 nucleotides in length. In certain embodiments, one or both of the ss-oligonucleotides may be more than 60 nucleotides in length. In certain embodiments, one or both of the ss-oligonucleotide and the second ss-oligonucleotide may be from about 10, 12, 14, 16, or 19 to about 20, 25, 30, 40, 50, 60, 100 or more nucleotides in length. In certain embodiments, one or both of the ss-oligonucleotide and the ss-oligonucleotide may be from about 20, 30, 40, or 50 to about 60, 80, 100, or more nucleotides in length.

Once formed, the ds-oligonucleotide containing the C-C or mC-C mismatch is contacted with Ag⁺. It is understood that a source Ag⁺ could be added at any point, for example before the two ss-oligonucleotides hybridize or after they have hybridized, as long as Ag⁺ is contacted with the ds-oligonucleotide containing the C-C or mC-C mismatch

In certain embodiments, the base pair mismatch is a C-C mismatch. In certain embodiments, the mismatch is an mC-C mismatch.

Certain embodiments are drawn to methods of detecting a cytosine-cytosine (C-C) base pair mismatch or a methylated cytosine-cytosine (mC-C) base pair mismatch in a double-stranded oligonucleotide (ds-oligonucleotide). The methods comprise reversibly binding Ag⁺ to the C-C or mC-C base pair mismatch. It has been discovered that Ag⁺ binding to C-C or C-mC base pair mismatch increases the hybridization stability of the ds-oligonucleotide. The increase in hybridization stability can be determined, for example, in comparison to hybridization stability in the absence of Ag⁺ reversible binding. This increase in hybridization stability can be determined by a number of different detection methods including, but not limited to, measuring the melting temperature, various optical measurements which distinguish between single- and double-stranded nucleic acids, various techniques based on the polymerase chain reaction such as qRT-PCR, nanopore detection, and various other electrical detection methods. Detection of increased hybridization stability of the ds-oligonucleotide in the presence of Ag⁺ is indicative of a C-C or mC-C base pair mismatch.

In certain embodiments, the increase in hybridization stability is detected using a nanopore or by using qRT-PCR. In certain embodiments, the increase in hybridization stability is detected using a nanopore. Although the methods of detecting a C-C base pair mismatch or a mC-C base pair mismatch in a double-stranded oligonucleotide (ds-oligonucleotide) may include detection using a nanopore or qRT-PCR, such methods are in no way meant to be limited to these detection methods.

In certain embodiments, detection of the increase in hybridization stability of the ds-oligonucleotide using a nanopore comprises applying a voltage to a sample containing the ds-oligonucleotide in a cis compartment of a duel chamber nanopore system wherein the voltage is sufficient to drive translocation of the hybridized ds-oligonucleotide through a nanopore of said system by an unzipping process and analyzing an electrical current pattern in the nanopore system over time, wherein the increased hybridization stability of the ds-oligonucleotide in the presence of reversible Ag⁺ binding produces an electrical current pattern that is different and distinguishable from an electrical current pattern produced by the ds-oligonucleotide in the absence of Ag⁺. The presence of reversible Ag⁺ binding to the mismatch may also produce an electrical current pattern that is different and distinguishable from an electrical current pattern produced by a ds-oligonucleotide with a different base pairing at the inter-strand lock site.

In certain embodiments, the C-C or mC-C base pair mismatch is within a hybridized region of at least 10 contiguous nucleotides. Although multiple base pair mismatches may reside within a hybridized region, in certain embodiments, at least 6, at least 7, at least 8, or at least 9 of the base-pairings within a contiguous hybridized region of at least 10 nucleotides are non-mismatched base-pairings. In certain embodiments, the hybridized region is a contiguous region of at least 11, at least 12, at least 13, at least 14, at least 15, at least 16, at least 17, at least 18, or at least 19 nucleotides. In certain embodiments, the hybridized region is a contiguous region of up to about 20 nucleotides, about 30 nucleotides, about 40 nucleotides, or about 50 nucleotides. In certain embodiments, the hybridized region is a contiguous region of more than 50 nucleotides. In certain embodiments, the hybridized region is a contiguous region of between about 10, 12, 14, or 16 to about 20, 25, 30, 40, 50, 60, 100, or more nucleotides. In certain embodiments, the hybridized region is a contiguous region of between about 20, 25, 30, 40, or 50 to about 60, 80, 100, or more nucleotides.

In certain embodiments, the ds-oligonucleotide is formed from two single-stranded oligonucleotides before or while the hybridization stability of the double-stranded oligonucleotide is increased. Such methods comprise hybridizing a first single-stranded oligonucleotide to a second single stranded oligonucleotide to form the ds-oligonucleotide comprising the C-C or mC-C base pair mismatch. In certain embodiments, one or both the first ss-oligonucleotide and the second ss-oligonucleotide comprise oligonucleotides of at least 10, at least 11, at least 12, at least 13, at least 14, at least 15, or at least 16 nucleotides in length. In certain embodiments, one or both of the ss-oligonucleotide and the second ss-oligonucleotide may be up to about 20 nucleotides in length, about 30 nucleotides in length, about 40 nucleotides in length, about 50 nucleotides in length, or about 60 nucleotides in length. In certain embodiments, one or both of the ss-oligonucleotides may be more than 60 nucleotides in length. In certain embodiments, one or both of the ss-oligonucleotide and the second ss-oligonucleotide may be from about 10, 12, 14, 16, or 19 to about 20, 25, 30, 40, 50, 60, 100 or more nucleotides in length. In certain embodiments, one or both of the ss-oligonucleotide and the ss-oligonucleotide may be from about 20, 30, 40, or 50 to about 60, 80, 100, or more nucleotides in length.

Once formed, the ds-oligonucleotide containing the C-C or mC-C mismatch is contacted with Ag⁺. It is understood that a source Ag⁺ could be added at any point, for example before the two ss-oligonucleotides hybridize or after they have hybridized, as long as Ag⁺ is contacted with the ds-oligonucleotide containing the C-C or mC-C mismatch.

In certain embodiments, the base pair mismatch is a C-C mismatch. In certain embodiments, the mismatch is an mC-C mismatch.

Although it may be known that a certain nucleic acid molecule (for example a target oligonucleotide) comprises one or more cytosine residues, it may be useful to further determine whether those residues are methylated, hydroxymethylated, or un-methylated. Thus, certain embodiments are drawn to methods of discriminating between a cytosine residue, a methylcytosine residue, and a hydroxymethylcytosine residue in a target single-stranded oligonucleotide (ss-oligonucleotide) or in a target strand of a double-stranded oligonucleotide (ds-oligonucleotide).

In certain embodiments, the target ss-oligonucleotide or the target strand of the ds-oligonucleotide is hybridized with a probe molecule. In certain embodiments, the probe molecule comprises a cytosine residue in a position designed to form a C-C, mC-C, or hmC-C base pair the residue to be investigated. This hybridization forms an at least partially double-stranded target/probe oligonucleotide that comprises a cytosine residue base pair mismatched with an un-modified cytosine (C-C), or a cytosine residue base pair mismatched with a methylated cytosine (mC-C), or a cytosine base pair mismatched with a hydroxymethylated cytosine (hmC-C), depending on which type of cytosine residue is present in the target nucleic acid at the site of interest.

In certain embodiments, the base pair mismatch is within a hybridized region of at least 10 contiguous nucleotides. Although multiple base pair mismatches may reside within a hybridized region, in certain embodiments, at least 6, at least 7, at least 8, or at least 9 of the base-pairings within a contiguous hybridized region of at least 10 nucleotides are non-mismatched base-pairings. In certain embodiments, the hybridized region is a contiguous region of at least 11, at least 12, at least 13, at least 14, at least 15, at least 16, at least 17, at least 18, or at least 19 nucleotides. In certain embodiments, the hybridized region is a contiguous region of up to about 20 nucleotides, about 30 nucleotides, about 40 nucleotides, or about 50 nucleotides. In certain embodiments, the hybridized region is a contiguous region of more than 50 nucleotides. In certain embodiments, the hybridized region is a contiguous region of between about 10, 12, 14, or 16 to about 20, 25, 30, 40, 50, 60, 100, or more nucleotides. In certain embodiments, the hybridized region is a contiguous region of between about 20, 25, 30, 40, or 50 to about 60, 80, 100, or more nucleotides.

Once formed, the ds-oligonucleotide containing the C-C, mC-C, or hmC-C mismatch is contacted with Ag⁺. It is understood that a source Ag⁺ could be added at any point, for example before the two ss-oligonucleotides hybridize or after they have hybridized, as long as Ag⁺ is contacted with the ds-oligonucleotide containing the C-C, mC-C, or hmC-C mismatch. It has been discovered that wherein Ag⁺ reversibly binds the C-C base pair mismatch, and to a lesser degree reversibly binds the mC-C base pair mismatch, it does not significantly bind the hmC-T mismatch. Thus, the amount of the reversible binding of Hg²⁺ is detected, wherein the amount detected indicates whether the cytosine residue in the target ss-oligonucleotide or in the target strand of the ds-oligonucleotide is un-methylated, methylated, or hydroxymethylated.

As described elsewhere herein, reversible Ag⁺ binding to a C-C or mC-C base pair mismatch can increase the hybridization stability of a double-stranded nucleic acid molecule. This increase in hybridization stability can be determined, for example, in comparison to the hybridization stability of the molecule in the absence of Ag⁺. This increase in hybridization stability can be determined by a number of different detection methods including, but not limited to, measuring the melting temperature, various optical measurements which distinguish between single- and double-stranded nucleic acids, various techniques based on the polymerase chain reaction such as qRT-PCR, nanopore detection, and various other electrical detection methods. In certain embodiments, the increase in hybridization stability is detected using a nanopore or by using qRT-PCR. In certain embodiments, the increase in hybridization stability is detected using a nanopore according to method described elsewhere herein. Although the methods of determining whether a cytosine residue in a target single-stranded oligonucleotide (ss-oligonucleotide) or in a target strand of a double-stranded oligonucleotide (ds-oligonucleotide) is an un-methylated cytosine residue, a methylated cytosine residue, or a hydroxymethylated cytosine residue may include detection using a nanopore or qRT-PCR, such methods are in no way meant to be limited to these detection methods.

In certain embodiments, the target ss-oligonucleotide or target strand of the ds-oligonucleotide and the probe molecule comprise oligonucleotides of at least 10, at least 11, at least 12, at least 13, at least 14, at least 15, or at least 16 nucleotides in length. In certain embodiments, at least one may be up to about 20 nucleotides in length, about 30 nucleotides in length, about 40 nucleotides in length, about 50 nucleotides in length, or about 60 nucleotides in length. In certain embodiments, at least one may be more than 60 nucleotides in length. In certain embodiments, at least one may be from about 10, 12, 14, 16, or 19 to about 20, 25, 30, 40, 50, 60, 100 or more nucleotides in length. In certain embodiments, at least one may be from about 20, 30, 40, or 50 to about 60, 80, 100, or more nucleotides in length.

In certain embodiments, the target ss-oligonucleotide or target strand of the ds-oligonucleotide comprises a plurality of cytosine residues which may or may not be methylated or hydroxymethylated. Therefore, certain embodiments herein are drawn to methods of determining whether one or more of such cytosine residues are methylated, hydroxymethylated, or un-methylated. In certain embodiments, multiple probe molecules are utilized that hybridize with the target oligonucleotide. The probe molecules are able to differentiate the different cytosine residues by forming various base pair mismatches, thus allowing the determination at multiple potential methylation sites. In certain embodiments, different probe molecules may comprise distinct tag domains that allow their differentiation and therefore all for multiplex discrimination.

The following disclosed embodiments are merely representative. Thus, specific structural, functional, and procedural details disclosed in the following examples are not to be interpreted as limiting.

### EXAMPLES

### Example 1

Oligonucleotides, including all targets and probes, were synthesized and HPLC purified by Integrated DNA Technologies (Coralville, Iowa). They were dissolved in dd water to 1 mM and stored at −20° C. as stocks. The target and probe DNAs were mixed at desire concentrations. The mixture was heated to 90° C. for 5 minutes, then gradually cooled down to room temperature and stored at 4° C. until use.

1,2-diphytanoyl-sn-glycerophosphatidylcholine (DPhPC, Avanti Polar Lipids) was used to form a lipid bilayer membrane over a ˜150 μm orifice in the center of a 25-μm-thick Teflon film (Goodfellow) that partitioned between cis and trans recording solutions. (Shim, J. W., Tan, Q., & Gu, L. Q. Single-molecule detection of folding and unfolding of a single G-quadruplex aptamer in a nanopore nanocavity. Nucleic Acids Res 37, 972-982 (2009)). The recording solutions on each side of the bilayer contained KCl at a desired concentration and were buffered with 10 mM Tris (pH 8.0). α-hemolysin protein was added in the cis solution, from which the protein was inserted into the bilayer to form a nanopore. Target and probe DNAs and HgCl₂ solutions were released to the cis solution. The voltage was given from trans solution and cis solution was grounded. In this configuration, a positive voltage pulled the negatively charged DNA through the pore from cis to trans. The ion current through the pore were recorded with an Axopatch 200B amplifier (Molecular Device Inc., Sunnyvale, Calif.), filtered with a built-in 4-pole low-pass Bessel Filter at 5 kHz, and acquired with Clampex 10 software (Molecular Device Inc.) through a Digidata 1440 A/D converter (Molecular Device Inc.) at a sampling rate of 20 kHz. The single-molecule events were analyzed using Clampfit 9.0 (Molecular Device Inc.), Excel (MicroSoft) and SigmaPlot (SPSS) software. In addition to the DNA duplex signature blocks (˜10-100 ms), spike-like single-stranded DNA translocation events were observed (˜10-100 μs). These events were excluded from histogram construction and analysis. Data was presented as mean±SD of at least three independent experiments. The nanopore measurements were conducted at 22±2° C.

The bisulfite conversion for target DNAs was performed using the EZDNA Methylation-Gold Kit™ (ZYMO Research Corp.). Briefly, 10 μl of the target oligonucleotide sample (1 mM) were mixed with 10 μl water and 130 μl conversion reagent in a PCR tube. The PCR tube with the sample was placed in a thermal cycler, then heated at 98° C. for 10 minutes and 64° C. for 2.5 h. 600 μl M-binding buffer was added to a Zymo-Spin IC™ column, then the sample was loaded into the column. After the conversion reaction, the column was centrifuged at 10,000×g for 30 s, followed by washing with 100 μl wash buffer. After centrifuging for 30 s, 200 μl desulphonation buffer was loaded in the column and incubated at room temperature for 15-20 min. After incubation, the column was spun at 10,000×g for 30 s, followed by washing twice with 200 μl wash buffer and spinning for 30 s. Purified olignucleotides were eluted with 10 μl elution buffer.

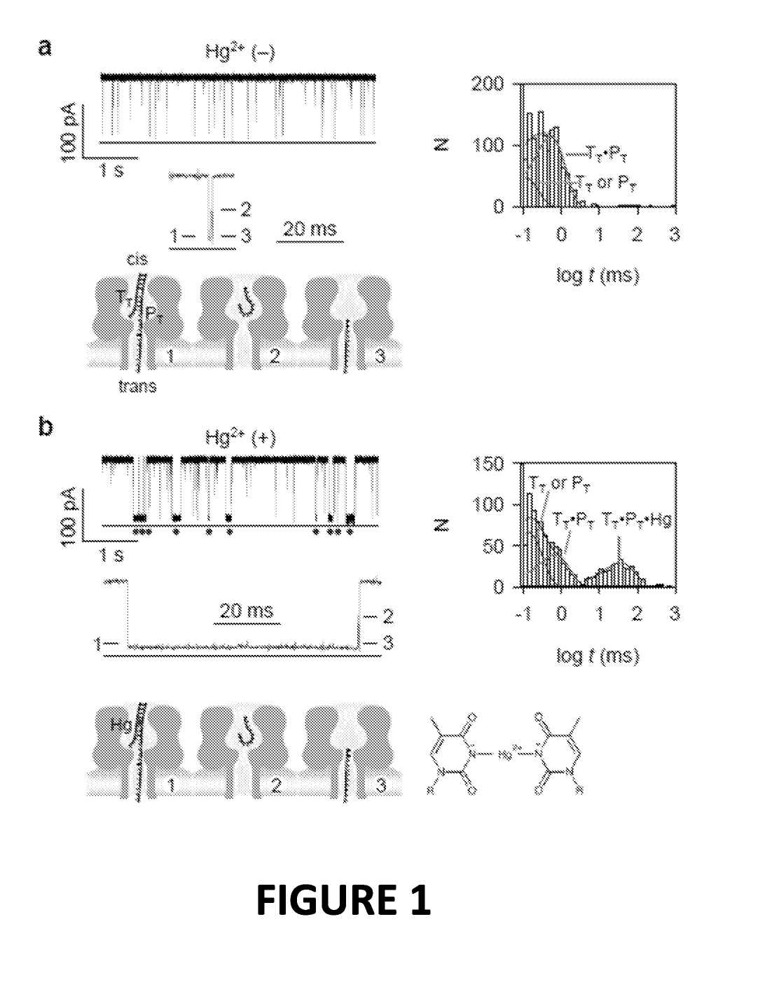

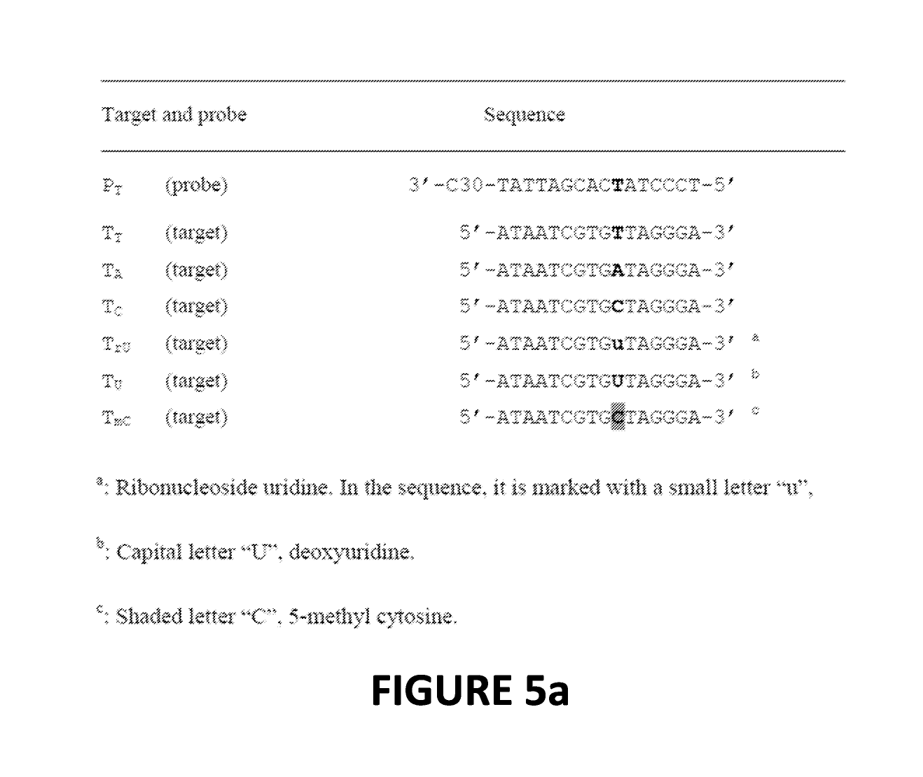

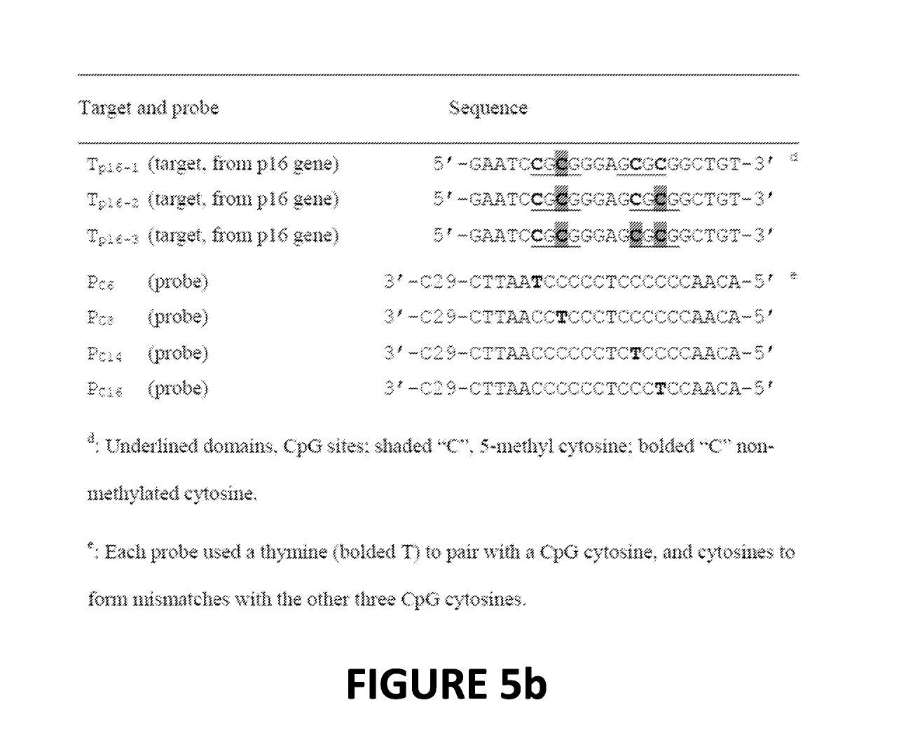

The 16-nucleotide single stranded target DNA T_{T} (SEQ ID NO: 2) and its single stranded probe P_{T} (SEQ ID NO: 1) (1 μM/1 μM) was presented to the cis side of the nanopore (see FIGS. 5a and 5b for sequences). The T_{T}·P_{T} hybrid formed a T-T mismatch at T10. P_{T} flanked a poly (dC)₃₀ tag at the 3′ end. As T_{T}·P_{T} was driven into the pore from cis entrance (Wang, Y., Zheng, D., Tan, Q., Wang, M. X., & Gu, L. Q. Nanopore-based detection of circulating microRNAs in lung cancer patients. Nat. Nanotechnol. 6, 668-674 (2011)), the tag threaded into the β-barrel, while the duplex domain was trapped in the nanocavity (FIG. 1a). The trapping of T_{T}·P_{T} generated a three-level conductance block (FIG. 1a). The block duration was 670±140 μs (+130 mV). As studied earlier, Level 1 of the block (IR/I=10%) is for T_{T}·P_{T} unzipping; Level 2 (IR/I=55%, ˜0.23 ms) is for T_{T} shortly residing in the nanocavity; and Level 3 (IR/I=11%, ˜0.12 ms) is for T_{T} translocating through the β-barrel. In addition to the T_{T}·P_{T} blocks, another type of short blocks with duration of 110±20 μs should be attributed to the free T_{T} or P_{T} that translocate through the pore.

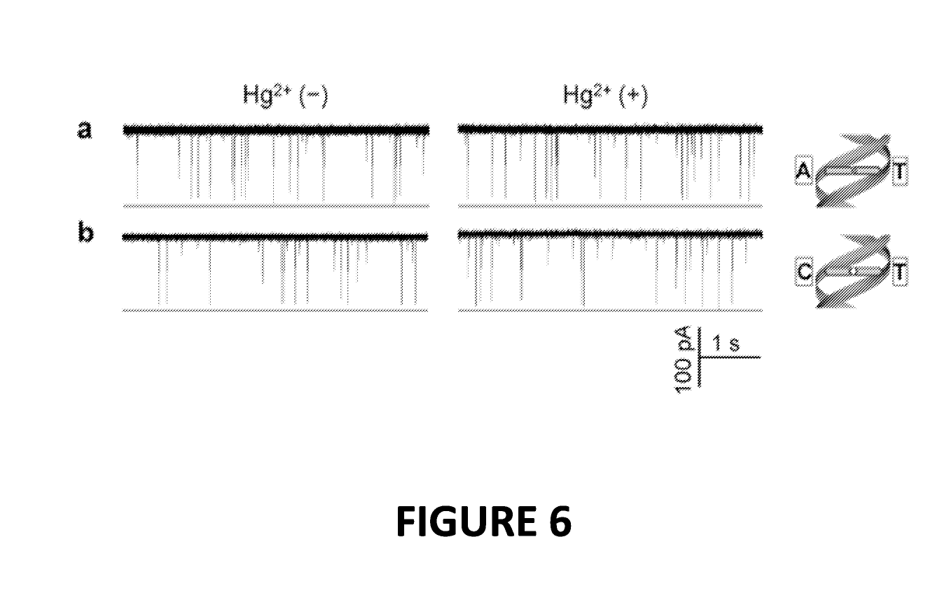

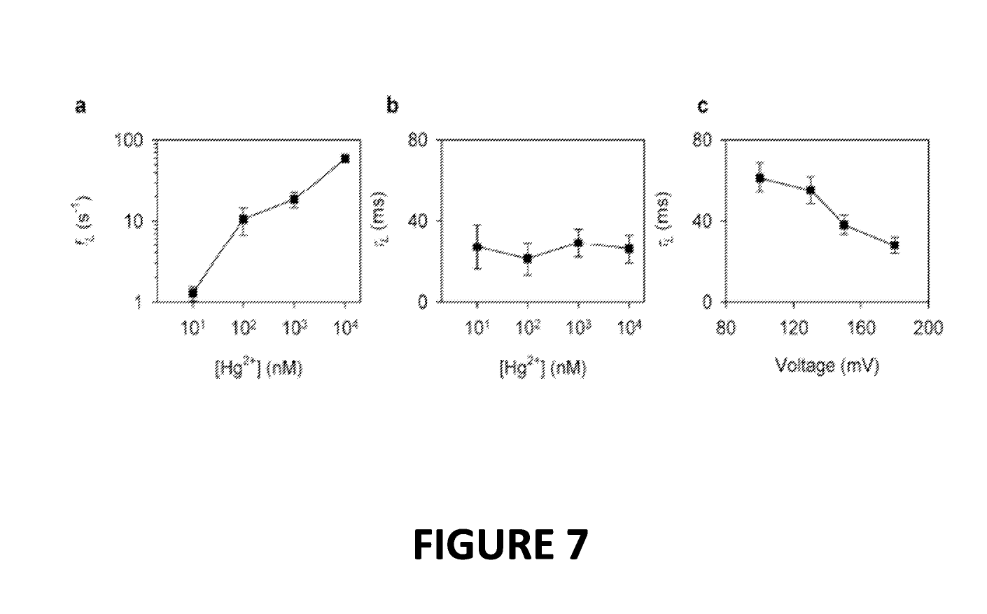

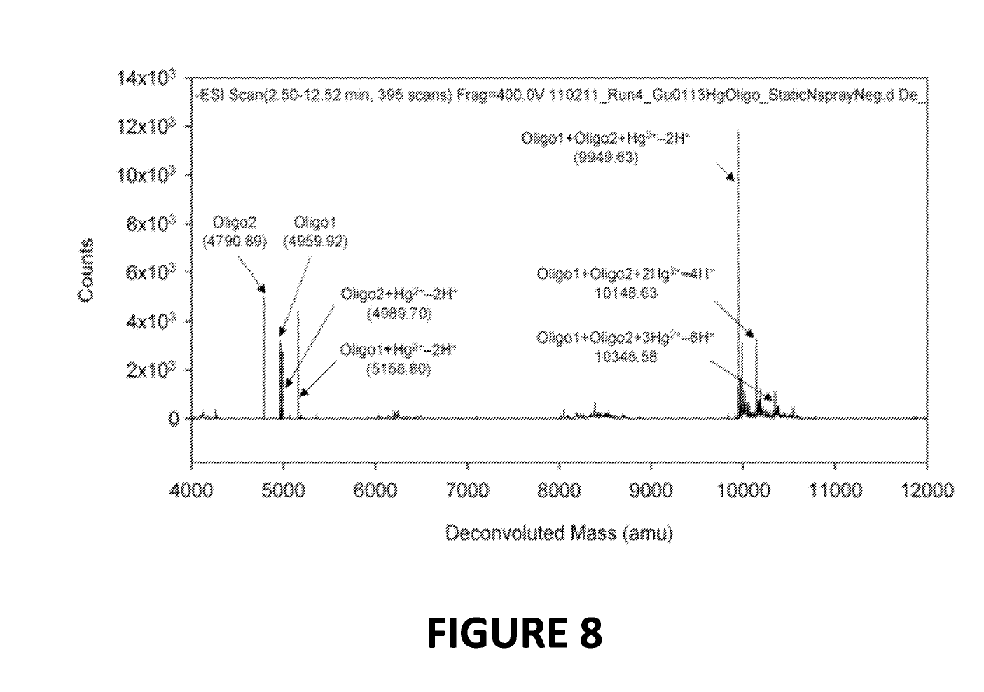

When HgCl₂ (10 μM) was added to cis solution, a new type of long three-level blocks appeared (FIG. 1b). They show similar Level 2 and Level 3 to the T_{T}·P_{T} signatures as in FIG. 1a. However, their Level 1 was prolonged over 50 folds, extending the entire block duration to 37±6 ms. These types of blocks were not observed for other types of mismatches such as cytosine-thymine (C-T) at the same position in the DNA duplex, whether in the presence or in the absence of Hg²⁺ ions (FIG. 6). Furthermore, the block frequency continuously increased with increasing the Hg²⁺ concentration in a broad range from 1 nM to 10 μM (FIG. 7a), while the block duration was independent to the Hg²⁺ concentration (FIG. 7b). These observations suggest the formation of the T_{T}·P_{T}·Hg complex. It was speculated that Hg²⁺ binds to the T-T mismatch of the T_{T}·P_{T} duplex to form a T-Hg-T bridge-pair. This motif greatly stabilized the complex, resulting in a 50-fold prolonged unzipping time. Increasing the voltage across the pore can effectively shorten the unzipping time from 62±7 ms at +100 mV to 28±3 ms at +180 mV (FIG. 7c). In addition, the mass spectrometry (MS) result shows a main component for Hg²⁺ binding to the dsDNA containing a T-T mismatch (FIG. 8). The removal of two H⁺ ions from the Hg²⁺/dsDNA complex is consistent with the predicted T-Hg-T structure (FIG. 1b). There were also minor peaks for Hg²⁺ binding with ssDNAs (FIG. 8). In the nanopore experiment, however, T_{T} or P_{T} alone only generated translocation blocks. It is uncertain whether Hg²⁺ binds to T_{T} or P_{T} in the nanopore detection, which is in different condition from the MS measurement (FIG. 8).

The equilibrium constant for the inter-strand lock can be evaluated by Kd=[T_{T}·P_{T}][Hg²⁺]/[T_{T}·P_{T}·Hg], where [T_{T}·P_{T}], [Hg²⁺] and [T_{T}·P_{T}·Hg] were concentrations of the three compounds. By comparing the block duration histograms in the absence (FIG. 1a) and in the presence of Hg²⁺ (FIG. 1b), the change in [T_{T}·P_{T}] can be evaluated, which was assumed to be [T_{T}·P_{T}·Hg]. Thus K_{d} was calculated to be 2.9 μM. Furthermore, the ratio of the T_{T}·P_{T}·Hg and T_{T}·P_{T} block duration (τ_{+Hg}/τ_{-Hg}) allows evaluating the energy increase for unzipping the T_{T}·P_{T}·Hg complex upon Hg²⁺ binding, ΔG=RT ln(τ_{+Hg}/τ_{-Hg})=8.1 kJ·mol⁻¹. Therefore, the T-Hg-T bridge-pair functions as an inter-strand lock, or MercuLock, that greatly stabilize dsDNA hybridization. The resulting nanopore signature can discriminate single T-T mismatches in a dsDNA.

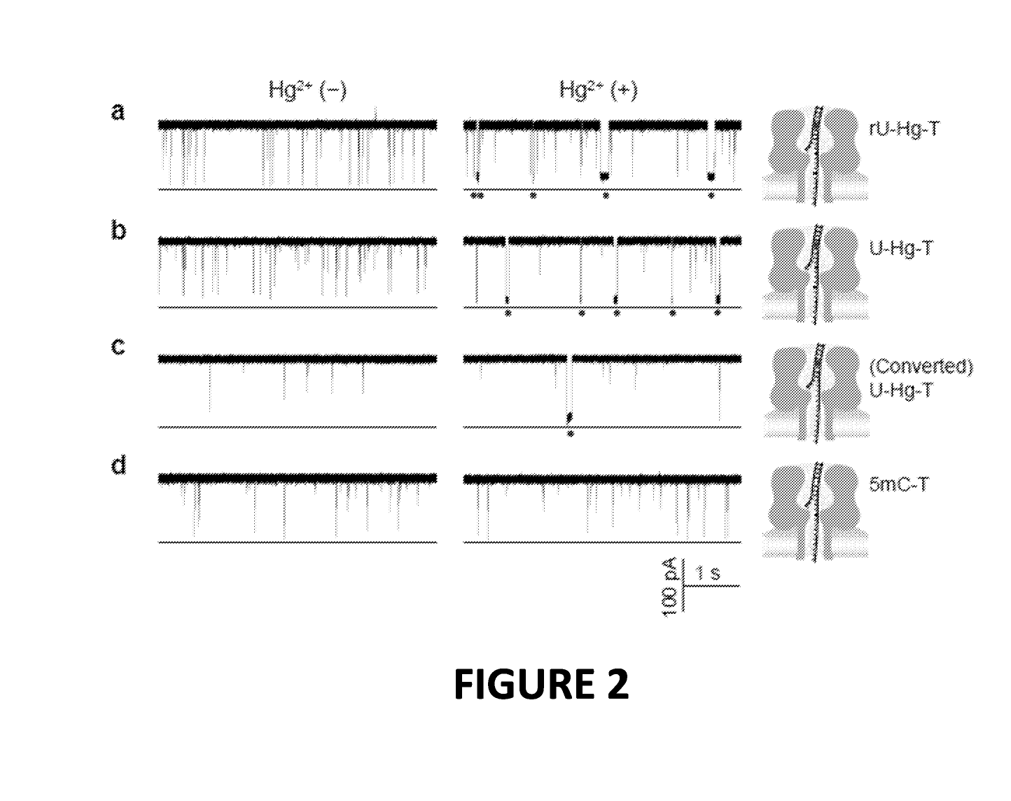

By utilizing the nanopore capability in single base-pair discrimination, it was further examined whether the Hg²⁺ inter-strand lock can be formed with mismatches other than T-T. The uracil-thymine (U-T) mismatch was examined because RNAs use uracil instead of thymine for complementary base pairing. The ss-oligonucleotide target T_{rU} (SEQ ID NO: 5) had one nucleotide difference from T_{T}, with T10 substituted by a ribonucleoside uridine (rU) (FIG. 5a). T_{rU} can be hybridized with the same probe P_{T} to form a rU-T mismatch. In the absence of Hg²⁺, the T_{rU}·P_{T} blocks were 820±110 ms (FIG. 2a left trace). The addition of Hg²⁺ to cis solution generated distinct long blocks of 41±6 ms (FIG. 2a right trace). This result is very similar to the T-T mismatch in the absence and in the presence of Hg²⁺ as in FIG. 1, suggesting that Hg²⁺ can bind the rU-T mismatch to form a stable rU-Hg-T inter-strand lock. Another target T_{U} (SEQ ID NO: 6), was tested which has a deoxyuridine (U, FIG. 5a) at the position T10. The T_{U}·P_{T} hybrid forms a U-T mismatch. It was discovered that Hg²⁺ can also form an inter-strand lock with the U-T mismatch (FIG. 2b). In the absence of Hg²⁺, short blocks (1.0±0.3 ms) were observed for T_{U}·P_{T} (FIG. 2b left trace), and in the presence of Hg²⁺ ions, a characteristic long block (39±5 ms) was identified that acts as a signature for the T_{U}·P_{T}·Hg complex (FIG. 2b right panel). Thus, Hg²⁺ forms an inter-strand lock with the uracil-thymine mismatch, which enhances the stability of the dsDNA by 40-50 times.

It is common in methylation detection to pre-treat DNA with bisulfite to convert cytosine into uracil. It was further examined whether uracil converted from cytosine can form an inter-strand lock with thymine. The target T_{C} (SEQ ID NO: 4), which has cytosine at the position 10, was treated with bisulfite; then the converted T_{C→U} and the probe P_{T} (not converted) were contacted and presented in cis solution. The current traces for converted T_{C→U}·P_{T} (FIG. 2c) are similar to T_{U}·P_{T} (FIG. 2b). The signature blocks for the T_{C→U}·P_{T} complex in the absence of Hg²⁺ was 1.3±0.2 ms (FIG. 2c left trace). The T_{C→U}·P_{T} complex in the presence of Hg²⁺ generated a long signature block with duration of 31±6 ms (FIG. 2c right panel). It was determined that Hg²⁺ did not bind the C-T mismatch in a T_{C}·P_{T} hybrid (FIG. 6). These findings confirm that cytosine has been converted to uracil and the inter-strand lock is formed between the cytosine-converted uracil and thymine. The dsDNA stability can be enhanced over 20 folds upon Hg²⁺ binding. Another target T_{mC} (SEQ ID NO: 7) was constructed that contained a 5′-methylcytosine in the same position. 5′methylcytosine cannot be converted by bisulfite treatment. In contrast to T_{C}, the T_{mC}·P_{T} complex did not produce the long signature blocks. Only short blocks were observed either in the absence (1.7±0.9 ms, FIG. 2d left trace) or in the presence (1.8±0.4 ms, FIG. 2d right trace) of Hg²⁺, confirming that 5′-methylcytosine does not form a tight inter-strand lock with thymine. Overall, single bases of uracil and 5′-methylcytosine can be discriminated or distinguished by identifying the presence or absence, respectively, of inter-strand lock formation in the nanopore. Without intending to be bound by theory, it is thought that since uracil is converted from unmethylated cytosine, in principle unmethylated cytosine can be distinguishable from 5′-methylcytosine in the original DNA sequence.

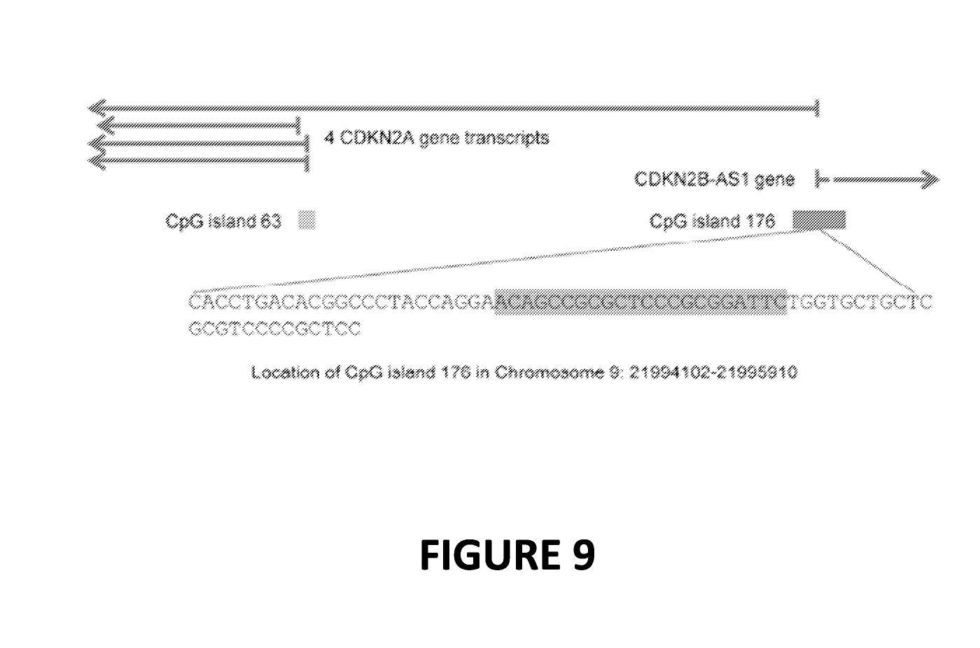

The p16 tumor suppressor gene (cyclin-dependent kinase inhibitor 2A, CDKN2A) performs an important role in regulating the cell cycle, and is a commonly studied target gene for cancer detection. The methylation status in the p16 gene has been known to be related to the risk of developing a variety of cancers such as lung cancer and breast cancer. In this illustrative example, the target was a 22-nt fragment from the antisense chain of the p16 gene within CpG island 176 (Chromosome 9: 21,994,825-21,994,846, FIG. 9). This fragment includes 4 CpGs in positions 6, 8, 14 and 16 (FIG. 5b). To target the bisulfite-converted sequence, we designed four probes, P_{C6} (SEQ ID NO: 11), P_{C8} (SEQ ID NO: 12), P_{C14} (SEQ ID NO: 13), and P_{C16} (SEQ ID NO: 14). Each probe employed a thymine to match one of CpG cytosines, and the four probes can detect all the four CpGs (6, 8, 14 and 16). In this experimental design, there was a technical issue: the high GC content (70%) in this DNA fragment strengthens the target/probe hybridization, prolonging its de-hybridization time for the DNA duplex containing an mC-T mismatch. This may affect the discrimination between the mC-T signatures and the U-Hg-T signatures. To solve this issue, three cytosines were introduced to each probe to form mismatches with the other three CpG cytosines of the target (FIG. 5b), whether or not the target is converted. This design can significantly shortened the complex block duration in the absence of Hg²⁺, thus greatly enhancing the capability to discriminate inter-strand lock signatures.

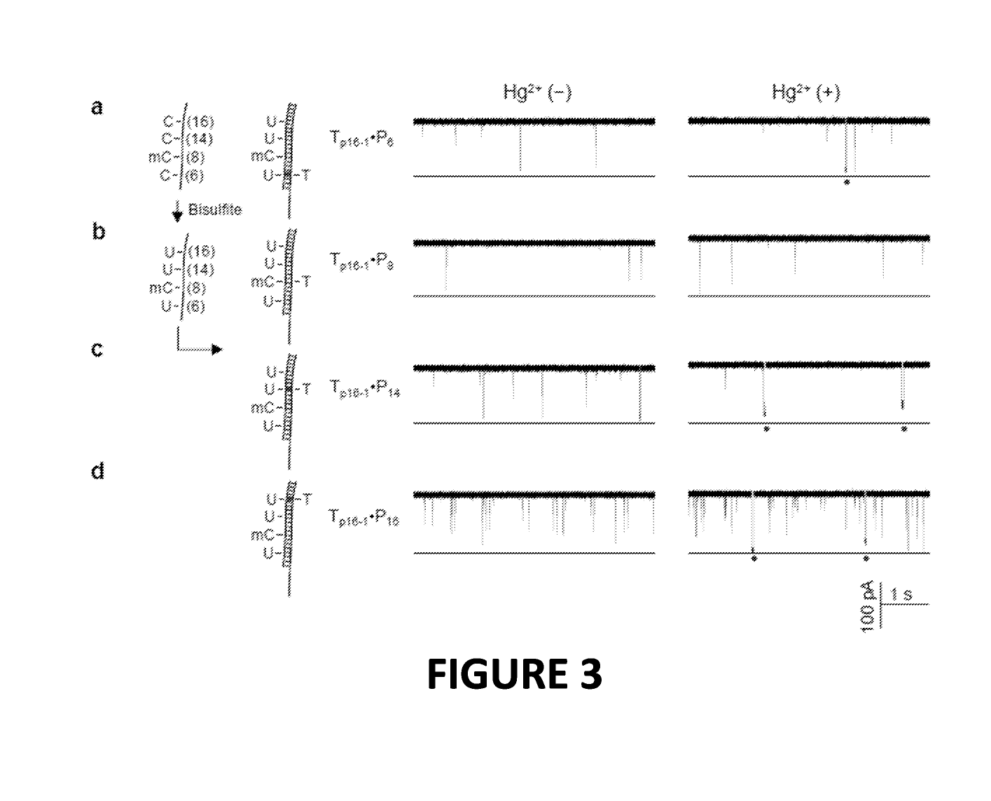

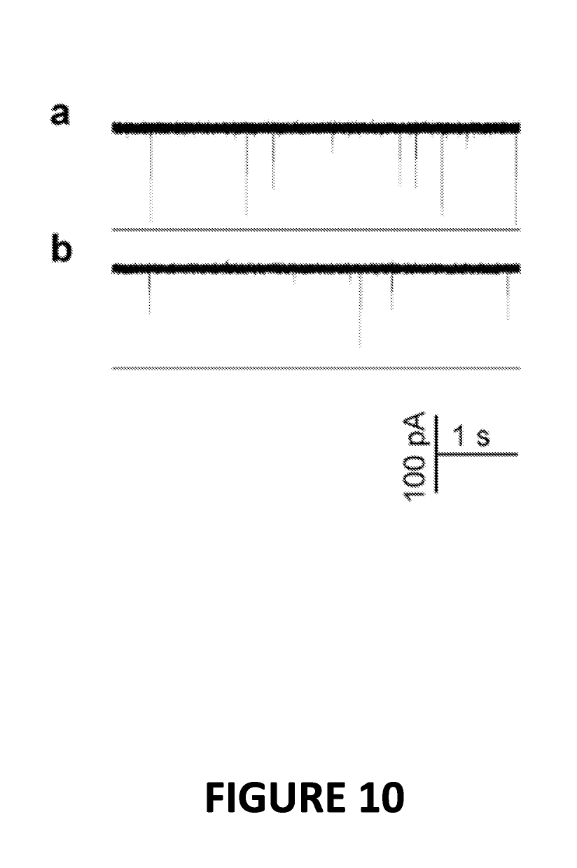

The target Tₚ₁₆₋₁ (SEQ ID NO: 8) comprises a 5′-methylcytosine at C8, and cytosines at C6, C14 and C16. The bisulfite-treated target Tₚ₁₆₋₁ was mixed with the four probes: P_{C6} (SEQ ID NO: 11); P_{C8} (SEQ ID NO: 12); P_{C14} (SEQ ID NO: 13); and P_{C16} (SEQ ID NO: 14), respectively. Their hybrids were detected in the nanopore individually. In a control experiment, Tₚ₁₆₋₁ alone before and after conversion only generated spike-like rapid translocation blocks (FIG. 10). FIG. 3a-d shows the current traces for the four mixtures in the absence and in the presence of Hg²⁺. In the absence of Hg²⁺, we only observed short blocks for all four mixtures (2.2-2.6 ms, FIG. 3a-d left traces). The addition of Hg²⁺ ions produced long blocks for the mixtures of converted Tₚ₁₆₋₁ and P_{C6} (11±6 ms, FIG. 3a right trace), P_{C14} (36±12 ms, FIG. 3c right trace) and P_{C16} (21±8 ms, FIG. 3d right trace). The only sample that did not generate the long signature block in Hg²⁺ was the mixture with P_{C8}. The distinct long blocks for P_{C6}, P_{C14} and P_{C16} are consistent with cytosines at C₆, C₁₄ and C₁₆, which have been converted to uracil to form the U-Hg-T inter-strand lock with the specific probe. In contrast, no long block signature observed in P_{C8} is in agreement with 5′-methylcytosine at C8 in Tₚ₁₆₋₁, that does not form the same inter-strand lock.

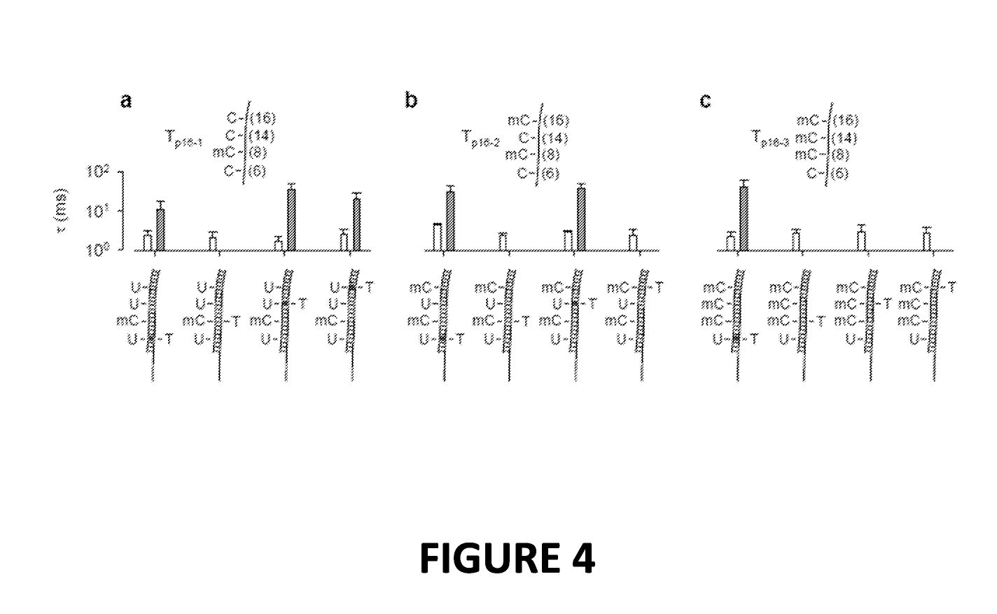

Targets carrying different numbers and distribution of 5mC were created. Tₚ₁₆₋₂ (SEQ ID NO: 9) has two 5′-methylcytosines at C8 and C16 and Tₚ₁₆₋₃ (SEQ ID NO: 10) has three at C8, C14 and C16 positions. Both of these targets have cytosines at other CpG sites as well. Each converted target was mixed with the four probes (the same probes used for Tₚ₁₆₋₁) respectively. Similar to Tₚ₁₆₋₁ (FIG. 4a), the hybrids of Tₚ₁₆₋₂ and Tₚ₁₆₋₃ with each of the four probes only produced short blocks (2.1-3.7 ms) in the absence of Hg²⁺. For Tₚ₁₆₋₂, the long block signatures can be observed with probes P_{C6} (32±11 ms) and P_{C14} (40±11 ms), and no such signature signals but only short blocks was observed with P_{C8} and P_{C16} in the presence of Hg²⁺ (FIG. 4b), verifying the formation of a U-Hg-T inter-strand lock between converted Tₚ₁₆₋₂·P_{C6} and Tₚ₁₆₋₂·P_{C14}, and no inter-strand lock formed for mCT mismatches in the Tₚ₁₆₋₂·P_{C8} and Tₚ₁₆₋₂·P_{C16} complexes. This result is consistent with the methylation distribution in Tₚ₁₆₋₂: cytosine at C6 and C14, and 5-methylcytosine at C8 and C14. Similarly, the mixture of converted Tₚ₁₆₋₃ with each of P_{C8}, P_{C14} and P_{C16} cannot generate the long block signatures, and only short blocks (2.3-2.8 ms) was observed. The long block signatures were only observed with P_{C6} (42±19 ms, FIG. 4c), thus verifying the methylation distribution in Tₚ₁₆₋₃: cytosine at C6 and 5-methylcytosine at C8, C14 and C16.

FIG. 1 shows the detection of a single T-Hg-T MercuLock in the nanopore. The mixture of target T_{T}, probe P_{T} were presented in cis solution. a and b. Representative current traces, multi-level signature blocks, duration histograms and diagram of molecular configurations, in the absence of Hg2⁺ (a) and in the presence of Hg²⁺ (b) panels were current traces showing multi-level block signatures produced by the T_{T}·P_{T} hybrid containing a T-T mismatch in the absence of Hg²⁺ (a) and in the presence of Hg²⁺ (b). Molecular configurations are provided at the bottom of the traces for multi-level blocks observed in a and b. a and b right panels were residual current-duration plots and block duration histograms constructed from current traces to the left. The sequences of target T_{T} and probe P_{T} are shown in FIG. 5a. Traces were recorded at +130 mV (cis grounded) in 1 M KCl buffered with 10 mM Tris (pH 7.4). cis solution contained 1 μM TT target and 1 μM P_{T} probe. In b, 10 μM HgCl₂ was presented in cis solution. Block duration values were given in Table 1. Dots under the trace in panel b marked the signature long blocks for the T_{T}·P_{T} hybrid bound a Hg²⁺ ion to the T-T mismatch. Dot in the model in panel b represent the MercuLock formed in the DNA duplex.

TABLE 1
Duration of the long and short types of blocks for different
base-pairs in the absence and in the presence of Hg^{2+a}

| | Bridging | Hg²⁺(−) | | Hg²⁺(+) | |
|---|---|---|---|---|---|
| Target•Probe | pair | τ_{S} (μs) | τ_{L} (ms) | τ_{S} (μs) | τ_{L} (ms) |
| T_{T}•P_{T} | T-Hg-T | 0.67 ± 0.14 | n.o.^{b} | 0.69 ± 0.12 | 37 ± 6 |
| T_{A}•P_{T} | A-T | 2.6 ± 0.6 | n.o. | 2.9 ± 0.5 | n.o. |
| T_{C}•P_{T} | C-T | 1.1 ± 0.2 | n.o. | 1.3 ± 0.5 | n.o. |
| T_{rU}•P_{T} | rU-Hg-T | 0.82 ± 0.11 | n.o. | 0.83 ± 0.21 | 41 ± 6 |
| T_{U}•P_{T} | U-Hg-T | 1.0 ± 0.3 | n.o. | 0.92 ± 0.21 | 39 ± 5 |
| T_{c→U}•P_{T}^{C} | U-Hg-T | 1.3 ± 0.2 | n.o. | 1.4 ± 0.6 | 31 ± 6 |
| TₘC•P_{T} | mC-T | 1.7 ± 0.9 | n.o. | 1.8 ± 0.4 | n.o. |

^{a}+130 mV · 1M KC1 and 10 mM Tris (pH 7.4).
^{C}“n.o.”, no observation.
^{d} T_{c→U•} bisulfite-converted from T_{c•} in which C was converted to U.

FIG. 2 shows discrimination of uracil and unmethylated cytosine with MercuLock. a through d current trace showing signature blocks produced by various target·probe hybrids T_{rU}·P_{T} (a), T_{U}·P^{T} (b), T_{C→U}·P_{T} (c) and T_{mC}·P_{T} (d) in the absence (left panel) and in the presence of Hg²⁺ (right panel). These hybrids contained a mismatch of uracil (uridine)-thymine (rU-T), uracil (deoxyuridine)-thymine mismatch (U-T), converted uracil-thymine (U-T), and 5-methylcytosine-thymine (mC-T), respectively. T_{C→U} was converted from target T_{C} by bisulfite. Dots under the traces marked the signature blocks for Hg²⁺ binding to the corresponding mismatches. Dots in models represented the MercuLock formed in the DNA duplex. The sequences of targets T_{U}, T_{U}, T_{C}, T_{mC} and probe P_{T} were shown in FIG. 5a. Traces were recorded at +130 mV in 1 M KCl solution buffered with 10 mM Tris (pH 7.4). cis solution contained 1 μM target DNAs and 1 μM P_{T}, and 10 μM HgCl₂ (right traces). The traces for T_{C}·P_{T} with and without Hg²⁺ were shown in FIG. 6. Values of block duration were given in Table 1.

FIG. 3 shows site-specific detection of DNA methylation with a MercuLock. Site-specific detection of DNA methylation with a MercuLock. a through d were current traces for the bisulfite converted Tₚ₁₆₋₁ (p16 DNA fragment original sequence shown in FIG. 9) hybridized with probes P_{C6} (a), P_{C8} (b), P_{C14} (c) and P_{C16} (d) (sequences shown FIG. 5b) in the absence of Hg²⁺ (left panel) and in the presence of Hg²⁺ (right panel). The four probes were designed for detecting CpG cytosines at the positions C6, C8, C14 and C16. C8 was 5-methyl cytosine (mC) and remained unchanged after bisulfite treatment. The other three positions were unmethylated cytosine (C) and thus converted to uracil (U) by bisulfite treatment. Dots under the traces marked the signature long blocks for Hg²⁺ ion binding to the U-T mismatches. Dots in the

models (left) marked the MercuLock in the DNA duplex.

FIG. 4 shows the detection of DNA containing different numbers and distribution of methylated cytosines. a, b and c compared the duration of short and long signature blocks for targets Tₚ₁₆₋₁ (a), Tₚ₁₆₋₂ (b) and Tₚ₁₆₋₃ (c) detected by four probes P_{C6}, P_{C8}, P_{C14} and P_{C16}. The duration of signature blocks allowed determining the methylation status for each of four CpG cytosines. The DNA sequences of the three p16 fragments were given in FIG. 5b. Duration values were given in Table 2. All traces were recorded at +130 mV in 1 M KCl and 10 mM Tris (pH 7.4).

TABLE 2
Duration of blocks for discriminating methylation status
at individual CpG sites in synthetic p16 gene fragments

| | | Methyl- | | | | |
|---|---|---|---|---|---|---|
| | CpG | ation | Hg²⁺(−) | | Hg²⁺(+) | |
| Target•Probe | site^{a} | Status^{b} | τ_{S} (ms) | τ_{L} (ms) | τ_{S} (ms) | τ_{L} (ms) |
| Tₚ₁₆₋₁•P₆ | 6 | C | 2.2 ± 0.5 | n.o. | 2.5 ± 0.9 | 11 ± 6 |
| Tₚ₁₆₋₁•P₈ | 8 | mC | 2.8 ± 0.4 | n.o. | 2.1 ± 0.8 | n.o. |
| Tₚ₁₆₋₁•P₁₄ | 14 | C | 2.4 ± 0.7 | n.o. | 1.7 ± 0.6 | 36 ± 12 |
| Tₚ₁₆₋₁•P₁₆ | 16 | C | 2.6 ± 0.5 | n.o. | 2.7 ± 0.8 | 21 ± 8 |
| Tₚ₁₆₋₂•P₆ | 6 | C | 3.7 ± 0.6 | n.o. | 4.4 ± 0.8 | 32 ± 11 |
| Tₚ₁₆₋₂•P₈ | 8 | mC | 2.7 ± 0.5 | n.o. | 2.5 ± 0.4 | n.o. |
| Tₚ₁₆₋₂•P₁₄ | 14 | C | 2.5 ± 0.4 | n.o. | 2.9 ± 0.6 | 40 ± 11 |
| Tₚ₁₆₋₂•P₁₆ | 16 | mC | 2.6 ± 0.7 | n.o. | 2.5 ± 1.1 | n.o |
| Tₚ₁₆₋₃•P₆ | 6 | C | 2.1 ± 0.6 | n.o. | 2.3 ± 0.8 | 42 ± 19 |
| Tₚ₁₆₋₃•P₈ | 8 | mC | 2.8 ± 0.5 | n.o. | 2.9 ± 0.7 | n.o. |
| Tₚ₁₆₋₃•P₁₄ | 14 | mC | 2.4 ± 0.9 | n.o. | 2.9 ± 1.6 | n.o. |
| Tₚ₁₆₋₃•P₁₆ | 16 | mC | 3.0 ± 0.9 | n.o. | 2.8 ± 1.1 | n.o |

^{a}Positions of CpG cytosines.
^{c} Shaded “mC”, 5-methyl cytosine. Other “C”s: non-methylated cytosine.

FIG. 6 shows no formation of MercuLock with fully matched adenosine-thymine pair (AT) and cytosine-thymine mismatch (C-T). a-b, Current traces showing that no long blocks were observed, thus no MercuLock was formed in fully matched hybrid T_{A}·P_{T} (a) and the hybrid T_{C}·P_{T} that contains a C-T mismatch (b) in the absence (left) and in the presence (right) of Hg²⁺. Sequences of targets T_{A} and T_{C}, and probe P_{T} are shown in FIG. 5a. Traces were recorded at +130 mV (cis grounded) in 1 M KCl buffered with 10 mM Tris (pH 7.4). The mixture of 1 μM target DNA and 1 μM probe were presented in cis solution (a and b). 10 μM HgCl₂ was added to cis solution to observe MercuLock formation (right panels). Block duration was calculated in Table 1.

FIG. 7 shows Hg²⁺ concentration- and voltage-dependent frequency and duration of long blocks for the T_{T}·P_{T} hybrid. a-b, Hg²⁺ concentration-dependent frequency (g) and duration (τL) of long blocks produced by T_{T}·P_{T} that form a MercuLock at the T-T mismatch. Data was obtained from traces recorded in 0.5 M/3 M KCl (cis/trans). Recording in asymmetric solutions increased the number of blocks at low Hg²⁺ concentration [Wanunu et al. Nat. Nanotech. 5, 160-165 (2010) and Wang et al. Nat. Nanotech. 6, 668-674 (2011)], and shortened the block duration compared with symmetric solutions (1 M KCl on both sides). c, Voltage-dependent long block duration for T^{T}·P^{T} with a MercuLock. Data was obtained from traces recorded in 1 M KCl and 10 mM Tris (pH7.4) in the presence of 10 μM HgCl₂. DNAs in all recordings were 1 μM.

FIG. 8 shows negative Ion Static Nanospray QTOF Mass Spectrum for dsDNA containing a T-T mismatched base pair in the presence of Hg²⁺. The reaction sample contained two oligodeoxynucleotides (10 μM each) that were annealed in the presence of HgCl₂ (5 μM). The annealing reaction was carried out in an aqueous solution containing 20% methanol and 20 mM ammonium acetate (pH 6.8). Initially, the samples were prepared according to the reference J. Phys. Chem B, 114, 15106-15112 (2010), which reported the use of an electrospray MS on an API 2000 (MDS-SCIEX) in the negative ion mode for detection of Hg²⁺-crosslinked oligodeoxynucleotide duplex. However, the oligonucleotides studied in the referenced report contained only 6 or fewer bases per strand. Furthermore, the design of the ion source of the Agilent 6520A to be used for the analysis of the sample in the Proteomics Center is not the same as that of the API 2000 MS. Therefore, some trial and errors occurred before the expected complex was finally detected. Because initially no complex was found in the submitted sample by negative ion Nanospray MS, the MS measurement procedure was improved, including 1) switching from static nanospray emitters with metal-coated tips to uncoated emitters, 2) setting the source Fragmentor voltage to the highest allowed level (400 V), and 3) replacing the sample solvent with 50 mM dimethylbutylammonium acetate (DMBAA, pH 7). These improvements enabled the detection of the complex by Nanospray MS. The result shows Mass Spectrometric evidence for the crosslinking of the oligonucleotides by Hg²⁺. The theoretical neutral masses of the most intense isotopes for main possible structures to be found are given below: 1) Oligo1, ATAATCGTGTTAGGGA (SEQ ID NO: 20): 4959.8767 Da; 2) Oligo2, TCCCTATCACGATTAT (SEQ ID NO: 21): 4790.8407 Da; 3) Oligo1+Oligo2+Hg2+−2H+: 9949.6720 Da.

FIG. 9 shows the location of tested CpG rich sequence in CDKN2A gene CpG island. Human CDKN2A gene generates 4 transcript variants which differ in their first exons (upper arrowed lines). The gene contains 3 exons. Encoded proteins function as inhibitors of CDK4 kinase important for cell cycle regulation and tumor suppression. This gene is frequently hypermethylated, mutated or deleted in a wide variety of tumors. There are 2 different CpG islands at their promoter regions. The first CpG island (CpG island 176) encompasses both CDKN2A and CDKN2B-AS1 genes. A segment of CpG rich sequence in the first CpG island was selected for testing (highlighted in green color in DNA sequence).

FIG. 10 shows current traces showing the translocation of the p16 gene fragment Tp16-1 and its bisulfite-converted sequence. Traces were recorded at +130 mV in 1 M KCl buffered with 10 mM Tris (pH7.4).

A novel metal ion-nucleic acid interaction at the single base-pair level has been uncovered. The core discovery is a Hg²⁺-bridged inter-stand lock that strongly and selectively stabilizes the T-T, rU-T and U-T mismatches. The resulting significant difference in dsDNA stability leads to accurate single-base discrimination between uracil and thymine, and eventually the discrimination between cytosine and methylated cytosine. Comparing with other methylation analysis methodologies, this approach is label-free and does not require DNA amplification and sequencing. The single-molecule recognition of inter-strand lock formation is rapid and specific, and therefore may have potential in methylation biomarker detection for diagnostics. Currently, each CpG site needs a specific probe and each nanopore measurement reads only one CpG site.

This detection mode is suitable for single locus DNA methylation detection. It may also be used for genome-wide DNA methylation profiling with a high throughput nanopore platform.

### Example 2

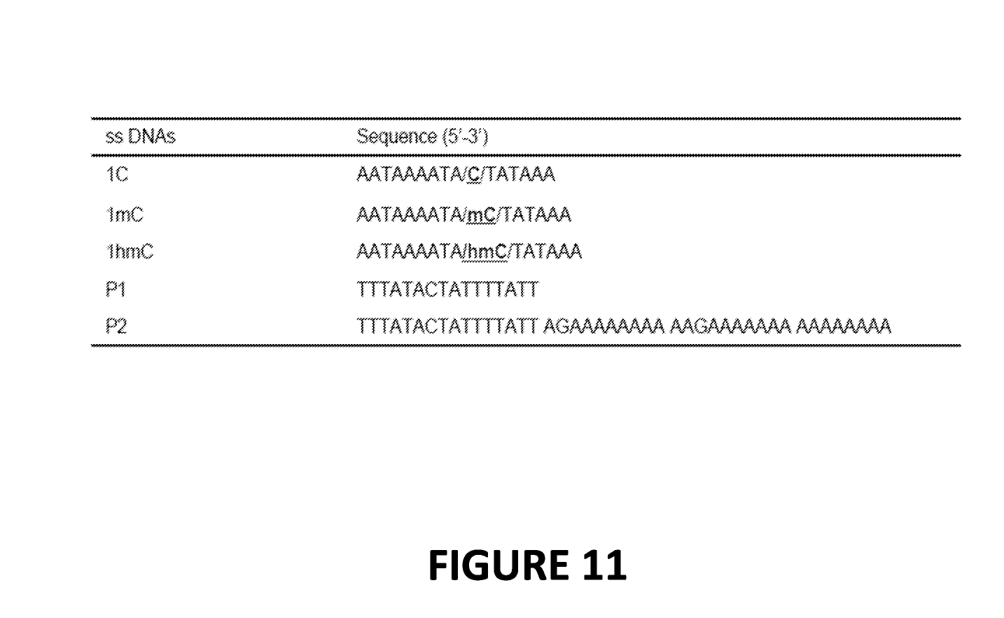

Electrophysiology setups and nanopore experimental methods are known in the art. Briefly, the recording apparatus was composed of two chambers (cis and trans) that were partitioned with a Teflon film. A planar lipid bilayer of 1,2-diphytanoyl-sn-glycerophosphatidylcholine (Avanti Polar Lipids) was formed spanning a 100-150 μm hole in the center of the partition. α-hemolysin (αHL) protein monomers (Sigma, St. Louis, Mo.) can be self-assembled in the bilayer to form molecular pores, which can last for hours during electrical recordings. Both cis and trans chambers were filled with symmetrical 1 M salt solutions (KNO₃) buffered with 10 mM 3-(N-morpholino)propanesulfonic acid (Mops) 8 and titrated to pH 7.02. All solutions were filtered before use. DNA oligonucleotides (FIG. 11) were synthesized and electrophoresis purified by Integrated DNA Technologies (IDT), IA. Before testing, the mixtures of DNA and probe were heated to 90° C. for 5 minutes, then slowly cooled to room temperature. Single-channel currents were recorded with an Axopatch 200A patch-clamp amplifier (Molecular Device Inc., former Axon Inc.), filtered with a built-in 4-pole low-pass Bessel Filter at 5 kHz, and acquired with Clampex 9.0 software (Molecular Device Inc.) through a Digidata 1332 A/D converter (Molecular Device Inc.) at a sampling rate of 20 kHz·s⁻¹. Data were based on at least four separate experiments and obtained by single channel search. The histograms were fitted by exponential log probability or Gaussian function, where appropriate. The triangles in each figure represent the capturing of DNA duplex in the nanopore. The electrophysiology experiments were conducted at 22±1° C. The ratio of Ag⁺ to DNA duplex was set to 100:1 in all the experiments. Varying the concentration of Ag⁺ (50×, 500×) does not change the number of DNA duplex capturing events significantly. This was similar to the previous findings that the melting temperature reached a plateau when the Ag⁺ concentration was 1.5 fold higher than the DNA. By isothermal titration calorimetry (ITC) and electrospray ionization mass spectrometry measurement, the binding of Ag⁺ to a DNA duplex containing a single C-C mismatches was identified at a 1:1 molar ratio 11, 12. The lines under each current trace mark the 0 current.

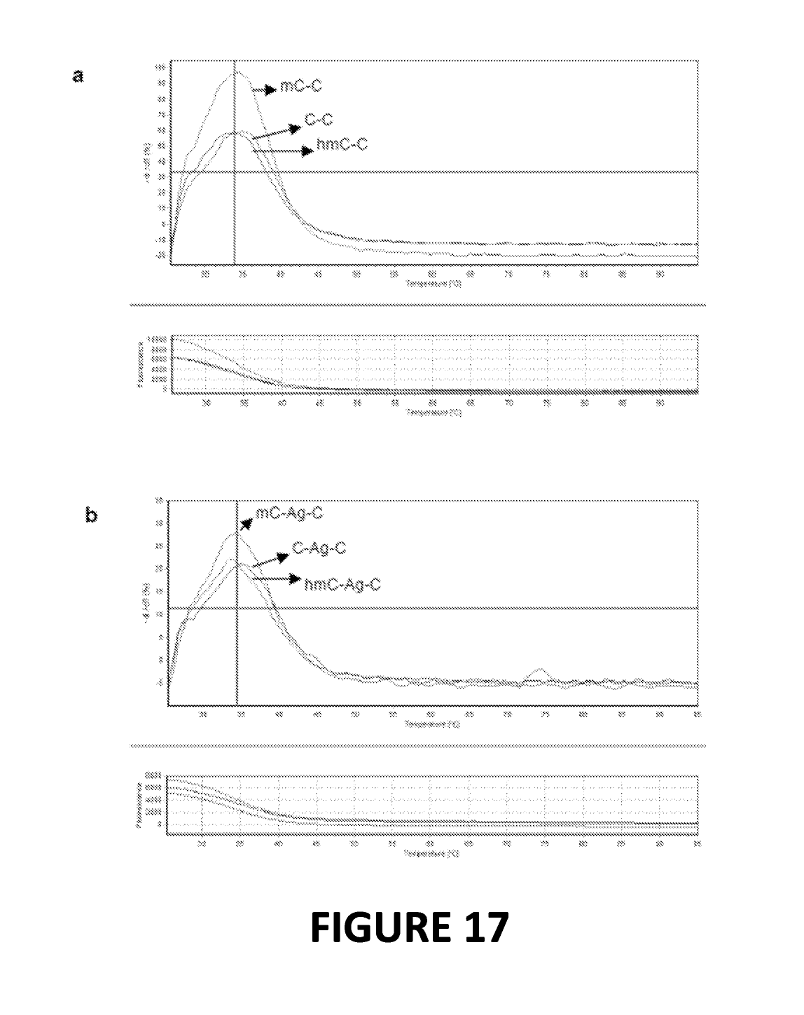

The Eppendorf Mastercycler® RealPlex² was used for Tm analysis and the fluorescence was monitored on SYBR Green I (Life Technologies), CA. Each solution consisted of 1 uM DNA duplex, 1 M KNO₃ and 25×SYBR Green at pH 7.02. Ag⁺ was 100 uM (50 uM Ag⁺ generate very similar results). The fluorescence curves (upper panel) and raw fluorescence curves (lower panel) for C-Ag-C, mC-Ag-C and hmC-Ag-C mismatches (FIG. 17b). The data shown in upper panels were the inverse of the differential of the curve shown in the lower panels in each figure, i.e., −dI/dT. The peak positions represent the Tm value.

The software NAMD was used to perform all-atom MD simulation on the IBM bluegene supercomputer. Force fields used in simulations were the CHARMM27 for DNA, the TIP3P model for water molecules, and the standard one for ions. Long-range coulomb interactions were computed using particle-mesh Ewald (PME) method. A smooth (10-12 Å) cutoff was used to compute the van der Waals interaction. After each simulation system was equilibrated at 1 bar, following simulations were carried out in the NVT (T=300 K) ensemble. The temperature of a simulated system was kept constant by applying the Langevin dynamics on Oxygen atoms of water molecules.

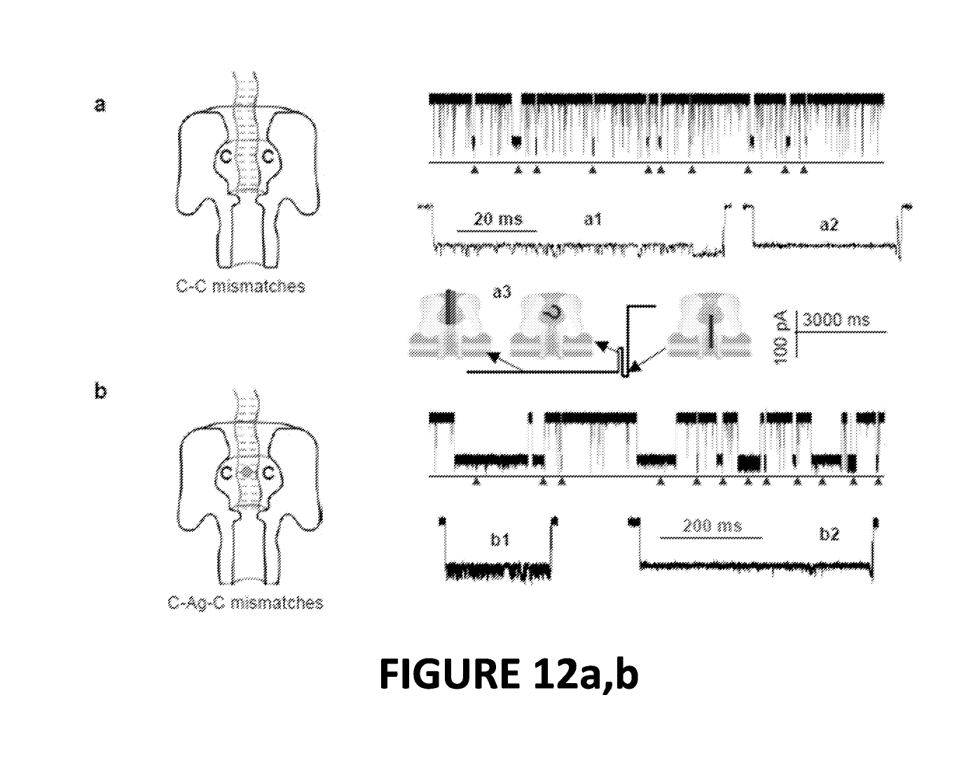

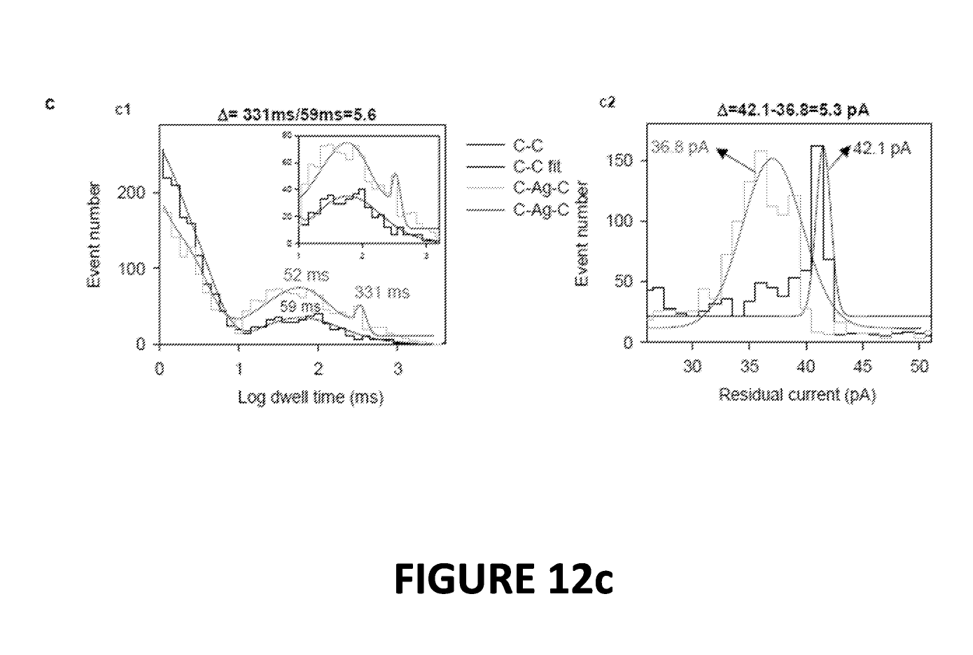

The addition of Ag⁺ increases the stability of dsDNA containing a C-C mismatch, which leads to an increase in the complex's dwell time within the nanopore (FIG. 12). Hybrid sequences (e.g., 1C and P1) are shown in FIG. 11. The events with an ending spike were identified (FIG. 12a1, a2), indicating DNA duplex capturing and dissociation. These dwell time differences provide a key differentiator between C-C and C-Ag-C. C-C generated dwell times with a peak at 59 ms (FIG. 12c1), while C-Ag-C generated a dwell times with first peak of 52 ms and second peak of 331 ms (FIG. 12c1). This second peak demonstrates dwell times with C-Ag-C that are 5.6-fold longer than seen with C-C (FIG. 12c1). This suggests that the C-Ag-C complex is more stable due to the increased amount of time that it takes to dissociate within the pore. Additionally, the dwell time histograms can provide further evidence of increased stability beyond the location of peaks: the ratio of the area under the histograms from 10¹-10¹⁶ ms (represents dsDNA) versus the area from 10°-10¹ ms (represents ssDNA) was 16 and 69 for C-C and C-Ag-C, respectively (Table 3).

TABLE 3
Fitted Area of the histograms with and without Ag⁺.

| DNA duplexes | C-C | C-Ag-C | mC-C | mC-Ag-C | hmC-C | hmC-Ag-C |
|---|---|---|---|---|---|---|
| Area (10⁰-10¹ ms)^{a} | 898 | 656 | 997 | 813 | 1278 | 786 |
| Area (10¹-10^{3.6} ms)^{b} | 14775 | 45188 | 28042 | 31897 | 16792 | 11323 |
| Area ratios of | 16 | 69 | 28 | 39 | 13 | 14 |
| (10¹-10^{3.6} ms)/(10⁰-10¹ ms) | | | | | | |
| Ratio of (with Ag/without Ag) | 4.3 | | 1.4 | | 1.1 | |

^{a}represents the area of the histograms of ssDNAs;
^{b}represents the area of the histograms of DNA duplex.

This 4.3-fold increase in the dsDNA:ssDNA dwell time ratio provides further evidence that the addition of Ag⁺ causes the DNA to spend a larger proportion of time in its duplex form. Finally, the temperature (Tm) was measured to be 34.9° C. and 35.3° C. for C-C and C-Ag-C, respectively (Table 4, FIG. 17), which also suggests C-Ag-C is more stable than C-C.

TABLE 4
Melting temperature (Tm, ° C.) of the DNA duplexes with and without Ag⁺.

| DNA duplexes | C-C | mC-C | hmC-C | C-Ag-C | mC-Ag-C | hmC-Ag-C |
|---|---|---|---|---|---|---|
| | 34.9 | 33.8 | 33.4 | 35 | 34.3 | 33.7 |
| | 34.8 | 34.1 | 33.9 | 35.4 | 34.4 | 34.3 |
| | 34.8 | 34.4 | 32.7 | 35.7 | 34.4 | 33.7 |
| | 34.9 | 33.8 | 33.9 | 35 | 34.5 | 33.5 |
| AVE ± SD | 34.9 ± 0.1 | 34 ± 0.3 | 33.5 ± 0.6 | 35.3 ± 0.3 | 34.4 ± 0.1 | 33.8 ± 0.4 |

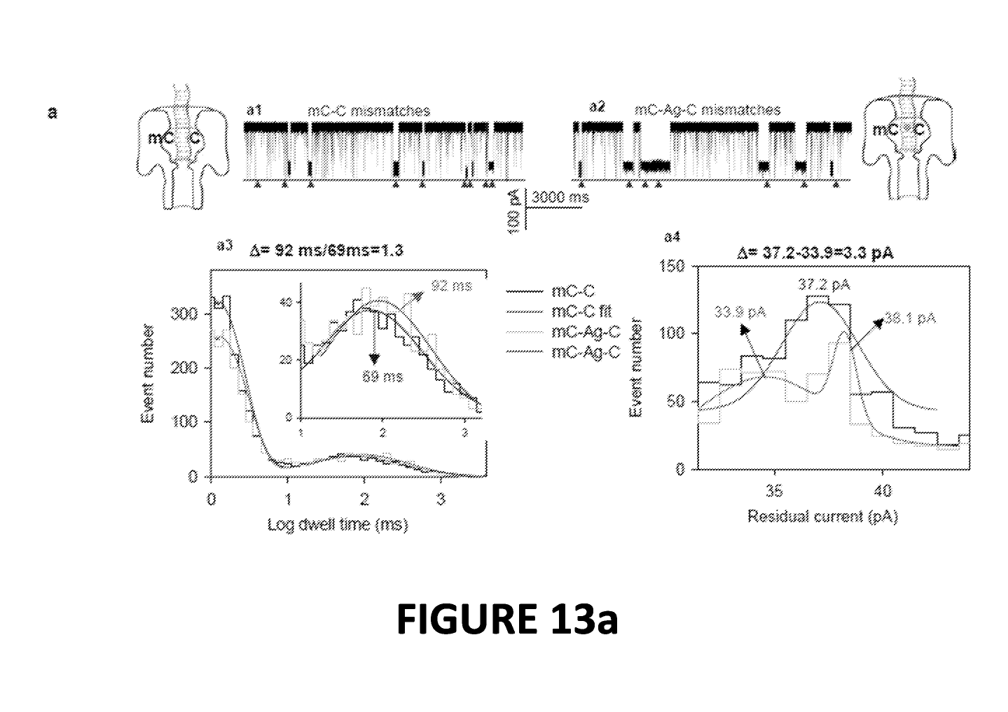

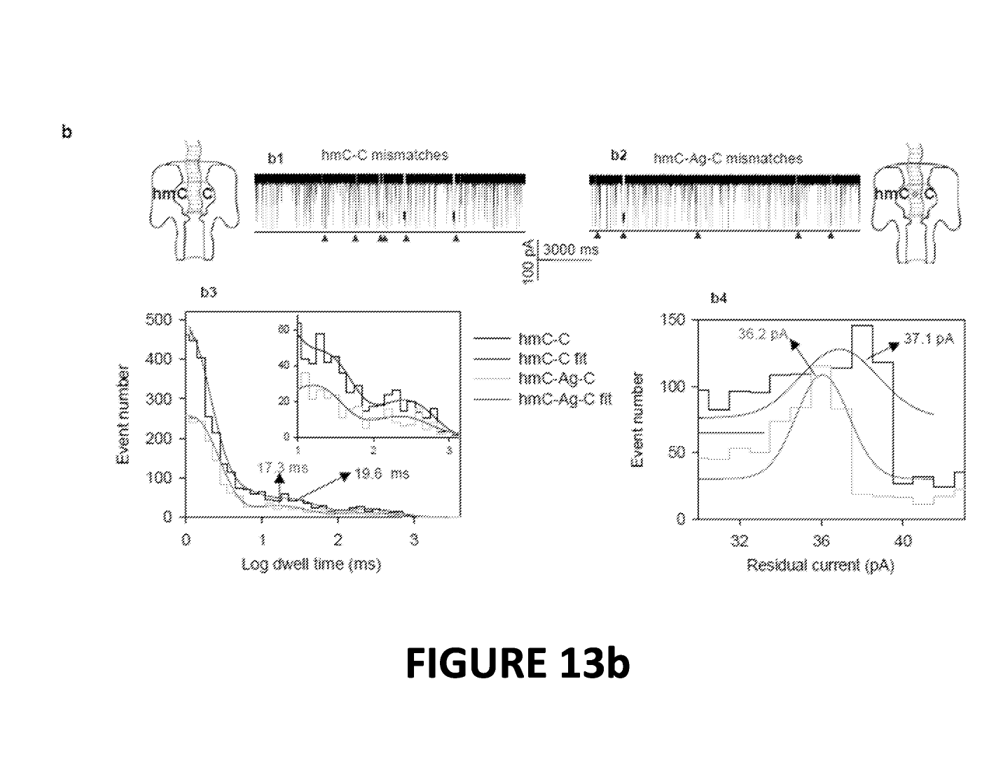

The addition of Ag⁺ also increases the stability of dsDNA containing an mC-C mismatch (1mC and P1 hybrids, FIG. 11), though the increases in stability and dwell time are less than with C-C (FIG. 13a1). It was found that mC-C generated a dwell time peak of 69 ms (FIG. 13a3), while mC-Ag-C generated a peak of 92 ms (FIG. 13a3), which represents a 1.3-fold increase. Once again, the ratio of area under the histograms from 10¹-10^{3.6} ms (represents dsDNA) versus the area from 10°-10¹ ms (represents ssDNA) increased with the addition of Ag⁺ from 28 (mC-C) to 39 (mC-Ag-C), for a change of 1.4-fold (Table 3). The melting temperature, Tm, was also found to change from 34.0° C. to 34.4° C. for mC-C and mC-Ag-C, respectively (Table 3, FIG. 17). These changes are superficial. Overall, all of these results suggest that Ag⁺ interacts poorly with mC-C.

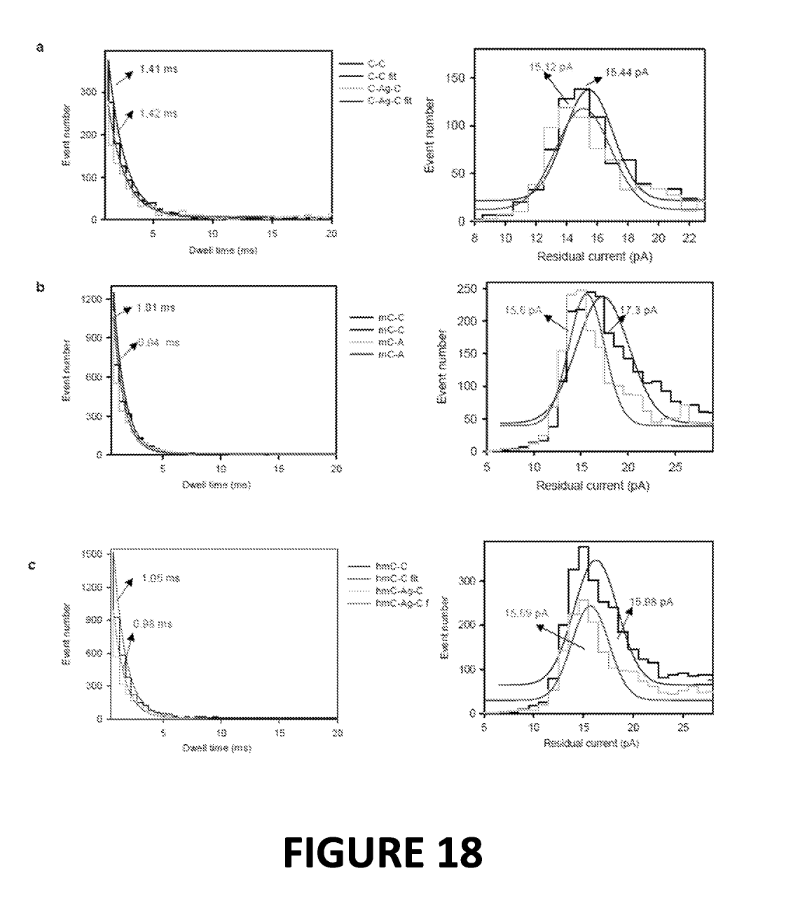

The addition of Ag⁺ does not appear to affect the stability of dsDNA containing an hmC-C mismatch (1hmC and P1 hybrids, FIG. 11), though stability and dwell time are less than with C-C and mC-C (FIG. 13b1). It was found that hmC-C generated a dwell time peak of 19.6 ms (FIG. 13b3), while hmC-Ag-C generated a peak of 17.3 ms (FIG. 13b3). Once again, the ratio of area under the histograms from 10¹-10^{3.6} ms (represents dsDNA) versus the area from 10⁰-10¹ ms (represents ssDNA) increased with the addition of Ag⁺ from 13 (mC-C) to 14 (mC-Ag-C), for a change of 1.4-fold (Table 3). The melting temperature, Tm, was also found to change from 33.5° C. to 33.8° C. for mC-C and mC-Ag-C, respectively (Table 4, FIG. 17). Overall, these data demonstrate the hmC-C mismatches are less stable than mC-C or C-C mismatches. Rather than provide stabilization, the presence of Ag⁺ seems to have little effect, and it is possible that Ag⁺ does not interact with hmC. Also, Ag⁺ doesn't interact with ssDNAs 1C, 1mC or 1hmC (FIG. 18).

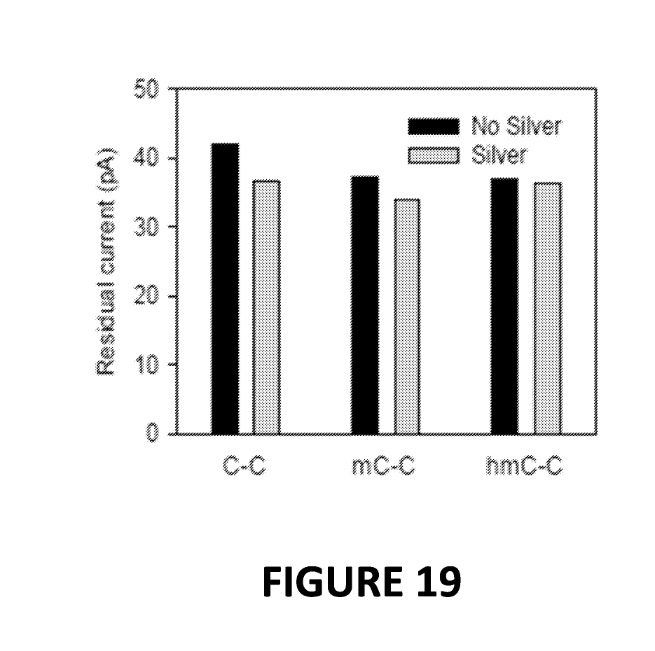

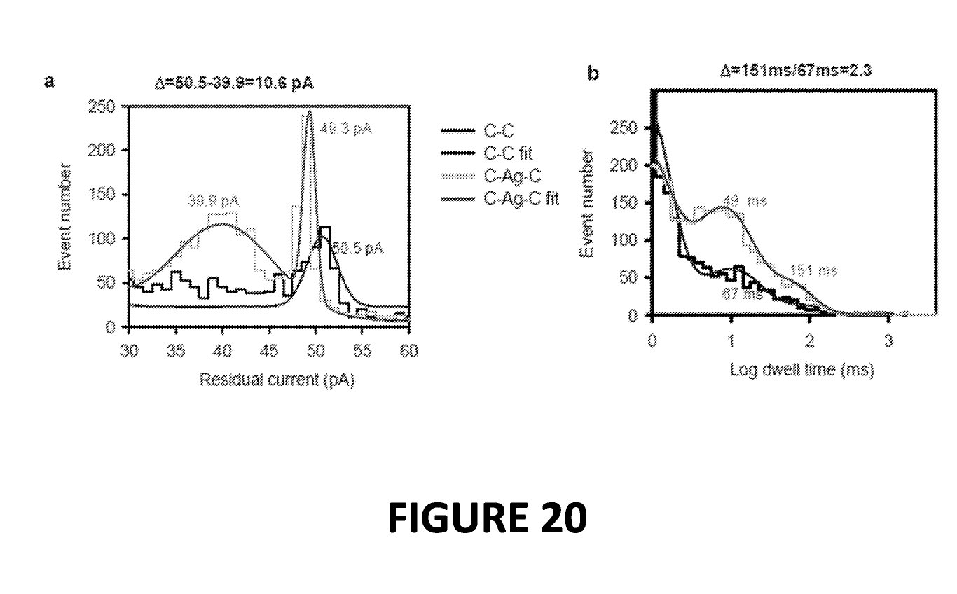

It was observed that the addition of Ag⁺ decreased the residual current at different degrees for C-C and mC-C mismatches (FIG. 19). C-C generated a peak of 42.1 pA (FIG. 12c2), but the C-Ag-C generated a peak of 36.8 pA (FIG. 12c2). The difference between C-C and C-Ag-C was 5.3 pA (FIG. 12c2). This difference increased to 10.6 pA at 180 mV (FIG. 20). mC-C generated a peak of 37.2 pA (FIG. 13a4). The mC-Ag-C generated two peaks of 33.9 pA and 38.1 pA (FIG. 13a4). The difference was about 3.3 pA between mC-C and the first peak of mC-Ag-C (FIG. 13a4). This also suggests the interactions between mC-C and Ag⁺ was weak. hmC-C generated a peak of 37.1 pA (FIG. 13b4). The hmC-Ag-C generated a similar peak of 36.2 pA (FIG. 13b4), which also suggests no stabilizing effect of Ag⁺ on hmC-C. Research demonstrated that the hydrated radius of Ag⁺ is 0.34 nm 16, which can block the ionic pathway at the pore constriction site. So it is reasonable to see a deeper current blockage with Ag⁺.

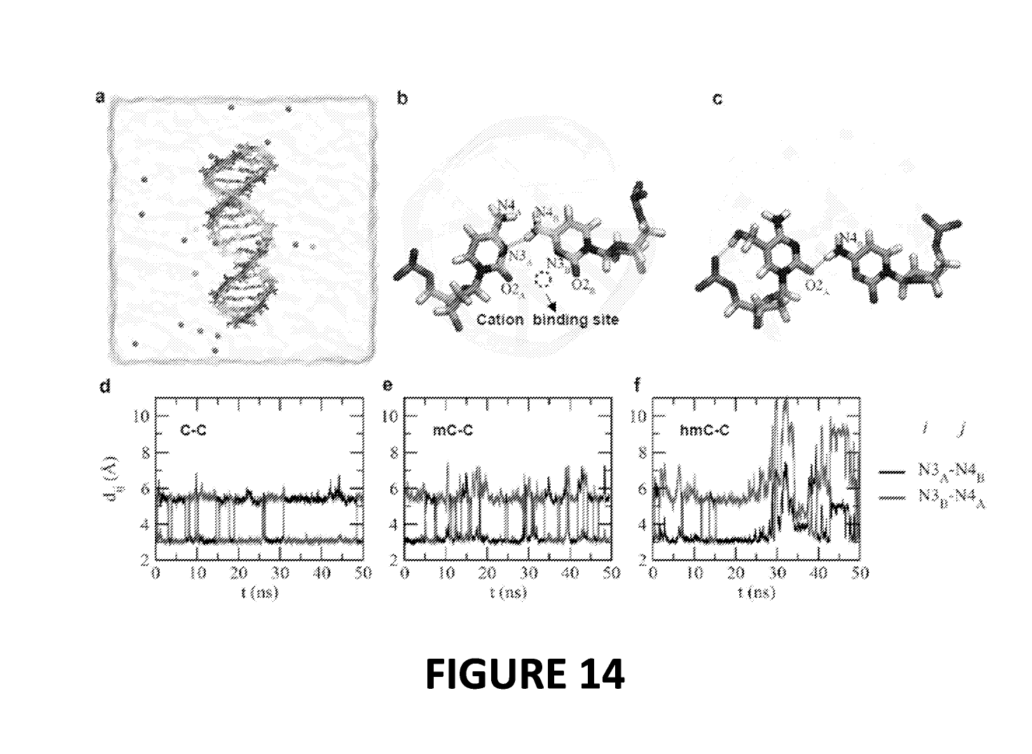

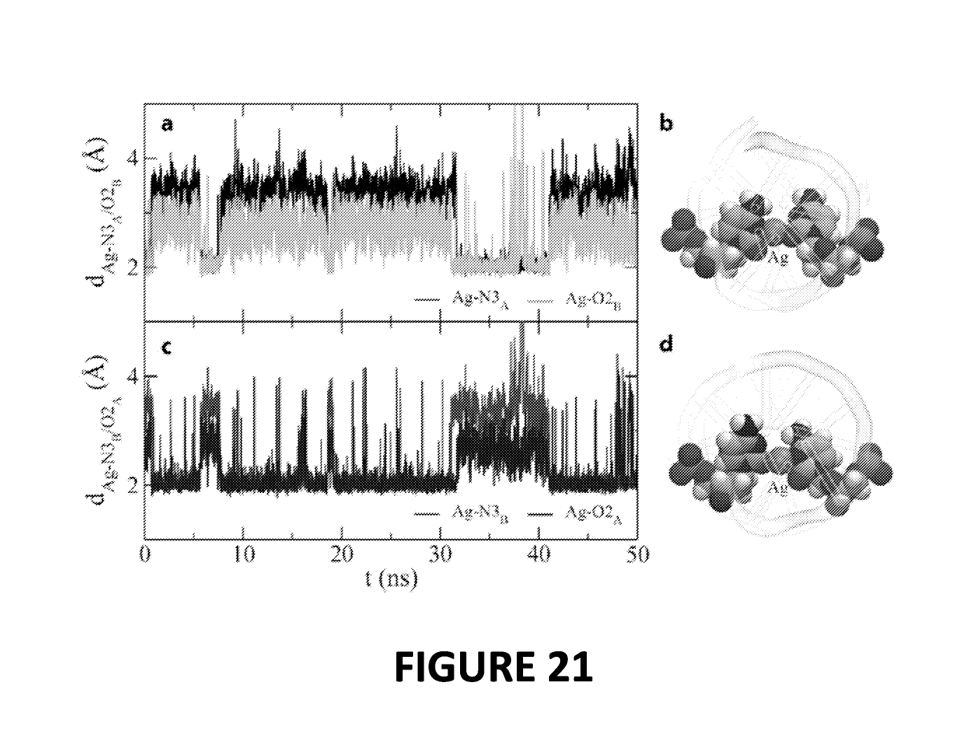

Molecular dynamics (MD) simulations of DNA duplexes containing these mismatches reveal how Ag⁺ may bind to the mismatches, and as well as different coordination configurations between the bases. As shown in FIG. 14a, a DNA duplex, having the same sequence as that in experiment was solvated in an electrolyte. The C-C base pairing was formed by the hydrogen bond between the N3 atom of one base and the N4 atom of the other base (FIG. 14b). Besides the conformation shown in FIG. 14b, another possible paring was formed by the hydrogen bond between N4A and N3B atoms. The distances between N3 and N4 atoms of different bases, as shown in FIG. 14d, indicate that hydrogen bonds are alternatively formed between N4A and N3B atoms and between N3A and N4B atoms. This type of pairing results in the formation of a binding site for a cation (FIG. 14b). During the simulation, K⁺ ions were found in the binding site and the mean residence time for K⁺ was about 10 ns. As confirmed in an independent MD simulation (FIG. 21), Ag⁺ can also enter the binding site and further stabilizes the paring between mismatched C-C bases. Correspondingly, both simulation and experimental results show that the dwell time of the duplex with a Ag⁺ was longer (FIG. 12c).

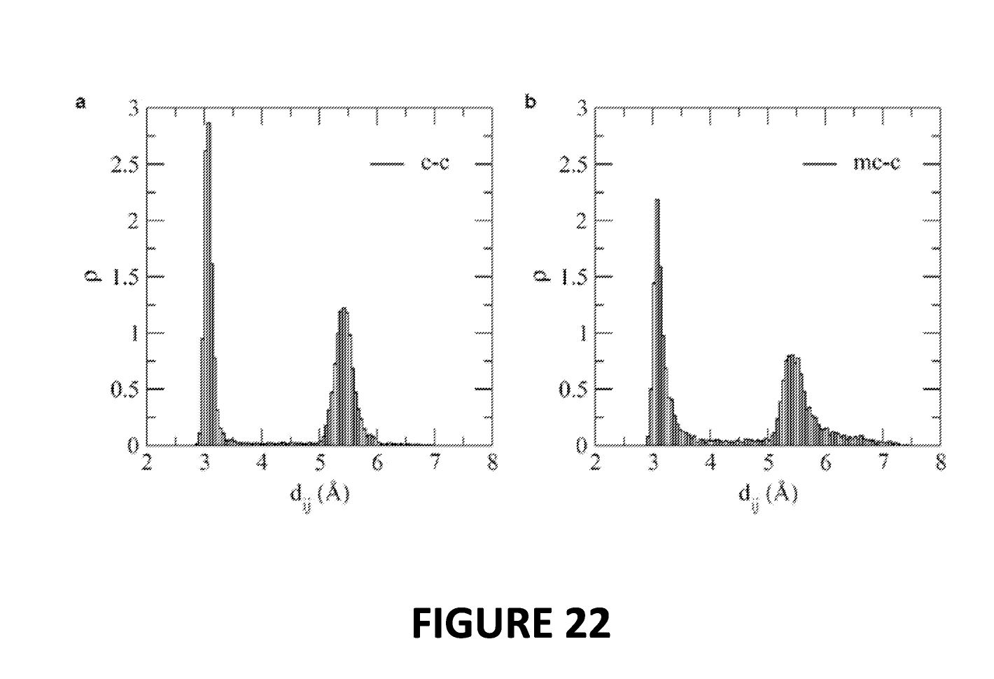

Simulations reflect experimental results for the differences in stability between the complexes. FIG. 14e shows that hydrogen bonds were formed and broken more frequently in mC-C compared to the C-C mismatch. Additionally, the probability for having longer bond lengths was higher for the mC-C than for the C-C mismatch (FIG. 22). Therefore, these results suggest that the cation binding site in the mC-C duplex was less stable than in the C-C duplex, consistent with the experimental results that the dwell time of C-Ag-C was longer than mC-Ag-C duplex (FIG. 12c1, FIG. 13a3) and that the Tm of C-Ag-C was higher than mC-Ag-C duplex (Table 4). Interestingly, for the duplex with the hmC-C, the base pairing was broken at about 25 ns during the simulation (FIG. 14f). Right before the breakage, FIG. 14c shows that, because of the hydrogen bond between the hydroxyl group in the hmC base and the phosphate group, the hmC base rotated towards the backbone of the duplex. Such interaction could also be mediated by a water molecule. In the meanwhile, base pairing was formed between the O2 atom in the hmC base and the N4 atom of the C base. After the breakage, the hmC and C bases can temporarily form inter-strand base-stacking, which causes the breakage of a neighboring basepair. Because the binding site falls apart in the duplex with the hmC-C mismatch, the effect of Ag⁺ on the dwell time should be negligible, as also demonstrated in nanopore experiments with hmC-C (FIG. 13b3) and Tm (Table 4). Overall, this shows tight agreement between the theoretical and experimental results.

Studies have found that Ag⁺ forms dinuclear complexes with cytosine and the complexes have been observed by X-ray diffraction. This study suggests that each of the methylcytosine residues doubly cross-linked by two Ag⁺ at the base binding sites N3 and O2. Thermodynamic properties of C-Ag-C complexes were studied by isothermal titration calorimetry (ITC) and circular dichroism (CD) and the results suggest that the specific binding between the Ag⁺ and the single C-C mismatched base pair was mainly driven by the positive dehydration entropy change of Ag⁺ and the negative binding enthalpy change from the bond formation between the Ag⁺ and the N3 positions of the two cytosine bases. However, our MD simulation of C-Ag-C shows that Ag⁺ is dynamically coordinated between N3A and O2B, or N3B and O2A (FIG. 14b, FIG. 21). This finding suggests that the coordination of Ag⁺ in C-Ag-C complexes may follow a different mechanism than previously thought.

The results confirm that Ag⁺ does in fact stabilize DNA duplexes containing C-C, with weaker interaction of Ag⁺ with DNA duplex containing mC-C. However, almost no interaction of Ag⁺ with DNA duplex containing hmC-C mismatches was observed. Different binding affinities for Ag⁺ ions with DNA duplexes containing C-C, mC-C or hmC-C could be explained in several ways. Firstly, by measuring the Tm, we also see a similar trend that {C, mC}-Ag-C (35.3 and 34.4° C.)>{C, mC}-C (34.9 and 34° C.), demonstrating that Ag⁺ coordination raises the melting temperature through the stabilization of C-Ag-C and mC-Ag-C, while the very similar Tm values for hmC-C (33.5° C.) and hmC-Ag-C (33.8° C.) indicate that Ag⁺ is not stabilizing hmC-Ag-C (Table 4, FIG. 18). Secondly, previous MD simulations found that H₂O molecules have the highest affinity for hmC when compared to C and mC, which increases the rotation probability. While our MD simulation revealed the water can mediate or direct interact with the phosphate group and the hydroxyl group in hmC. These results suggest a mechanism behind the lower stability of the basepairing in hmC-C mismatches. Thirdly, using atomic force microscopy (AFM), studies have found that the persistent length follows the trend mC>C>hmC 17, suggesting that hmC DNA has the largest flexibility and least structural stability. Finally, the —OH group in hmC can chelate with the phosphate group which may prevent stable hmC-Ag-C complex formation.

The discrimination of C, mC and hmC has been demonstrated using Ag⁺ and the α-HL nanopore platform. This offers improvement over the gold standard methodology for mC mapping, bisulfite conversion, in that all three cytosine forms can be distinguished simultaneously. Studies have found that C, mC or hmC can be recognized by immobilizing the DNA with streptavidin, chemical modifications in α-HL. While in a solid-state nanopore, studies found that DNA duplex contain mC and hmC can be discriminated, while C and mC can be discriminated by using methylated CpG binding proteins. Here it was demonstrated that C, mC and hmC can be discriminate successfully at the same time in both dwell time and residual current by utilizing the Ag⁺. This is a direct method needs no modification and amplification.

FIG. 12 shows that Ag⁺ stabilizes DNA duplex containing C-C mismatches. a, The capturing of C-C duplex (ssDNA 1C hybridized with P1) in the nanopore. b, The capturing of C-Ag-C in the nanopore, the blocks are longer than C-C duplex. c, the histogram of the dwell time in Log form (10¹-10³=10-1000 ms, c1). The C-C generated a single peak of 59 ms. The C-Ag-C generated two peaks of 52 ms and 331 ms, which increased the dwell time by 5.6 fold compare to C-C duplex. The right panel c2 shows the histogram of residual currents. The C-C generated a single peak of 42.1 pA; The C-Ag-C generated a peaks of 36.8 pA. The difference was 5.3 pA between C-C and C-Ag-C. The triangles indicate the capturing of DNA duplexes. The inset figures a1, a2, b1, b2 show the DNA duplex dissociation signature with an ending spike, and a3 shows the molecular configurations during the DNA duplex dissociation process. Recordings were made at 150 mV.

FIG. 13 shows interactions of Ag⁺ with DNA duplex containing mC-C and hmC-C mismatches. a, Weak interaction of Ag⁺ with DNA duplex contains mC-C mismatches (ssDNA 1mC hybridized with P1). The representative current traces of mC-C (a1) and mC-Ag-C (a2) capturing. a3, the histogram of the dwell time in Log form (10¹-10³=10-1000 ms). The mC-C generated a single peak of 69 ms. The mC-Ag-C generated a single peak of 92 ms, which increased the dwell time by 1.3 fold. a4, the histogram of residual currents. The mC-C generated a single peak of 37.2 pA; The mC-Ag-C generated two peaks of 33.9 pA and 38.1 pA. The difference was 3.3 pA between mC-C and mC-Ag-C duplex. b, No interaction of Ag⁺ with DNA duplex contains hmC-C mismatches (ssDNA 1hmC hybridized with P1). The representative current traces of hmC-C (b1) and hmC-Ag-C (b2) capturing. b3, the histogram of the dwell time in Log form (10¹-10³=10-1000 ms). The hmC-C generated a peak of 19.6 ms. The hmC-Ag-C generated a similar peak of 17.3 ms. b4, the histogram of residual currents. The hmC-C generated a peak of 37.1 pA; The hmC-Ag-C generated a similar peak of 36.2 pA. The triangles indicate the capturing of DNA duplexes. Recordings were made at 150 mV.

FIG. 14 illustrates molecular dynamics simulations of DNA duplex containing C-C, mC-C and hmC-C mismatches. A. Side-view of the simulation system. The DNA duplex is in the “stick” presentation and two backbones are illustrated. Potassium ions that neutralize the entire simulation system are shown. Water in a cubic box (78.5×78.5×78.5 Å3) is shown transparently. b. A snap-shot of pairing between two cytosine bases. The dashed circle highlights the binding site for a cation. c. A snap-shot of hmC-C pairing before the pairing was broken. d-f. Time-dependent distances between the N3 atom of one base and the N4 atom of the other base, in C-C (d), mC-C (e) and hmC-C (f) mismatches.

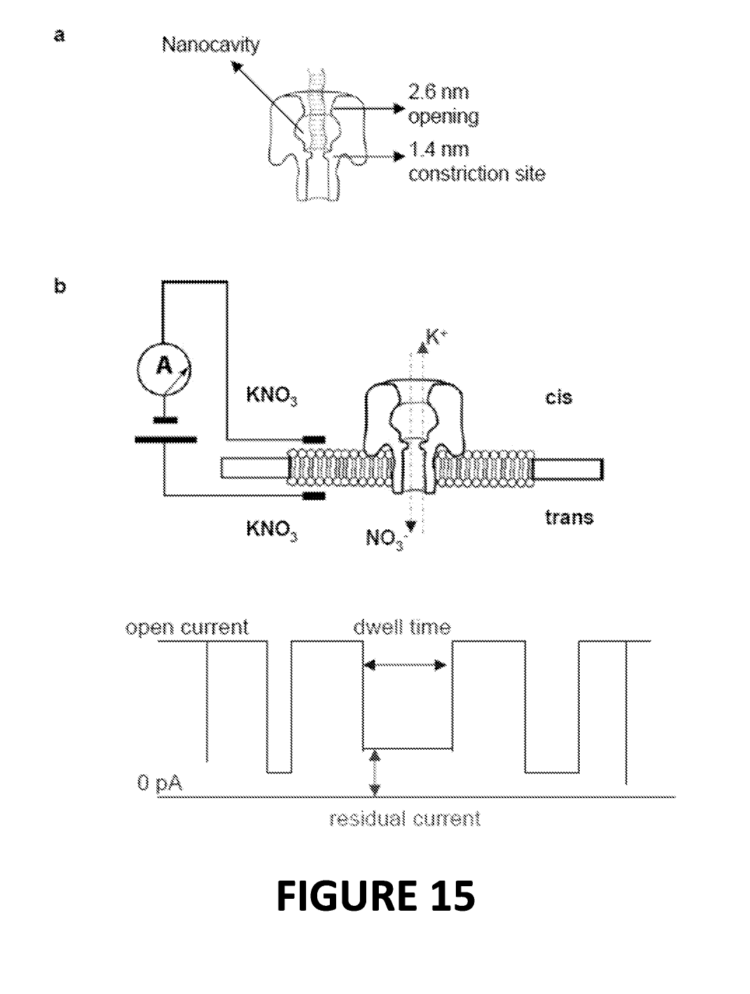

FIG. 15 illustrates the nanopore recording platform. a, the alpha-hemolysin nanopore has a nanocavity (2.6 nm opening and a 1.4 nm constriction site) can capture and hold the DNA duplex, b, during nanopore recording, a single α-HL nanopore is inserted into a lipid bilayer that separates two chambers (termed cis and trans) containing KCl buffer solution. Ionic current through the nanopore was carried by K⁺ and NO³⁻, ions, and a patch clamp amplifier applies voltage and measures ionic current. c, when a molecule interacts with the nanopore which will block the ionic pathway, then generate a “block” event. From the dwell time and residual current we can obtain meaningful information of the interactions between the molecule and the nanopore.

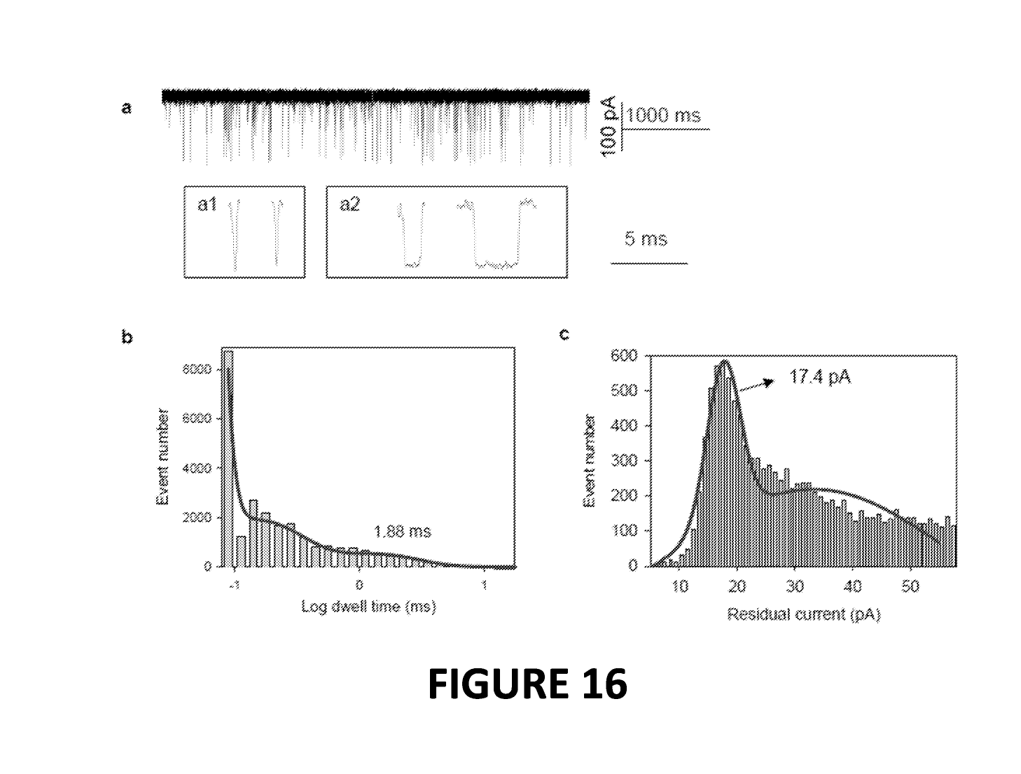

FIG. 16 shows that ssDNA P1 interacts with the nanopore. a, the representative current trace recorded at 150 mV. Two types of events were identified: a1: spike-like current profile which last about 200 us and a2, rectangular-like current profile which last about 1 to 10 ms. b, the histogram of the dwell time in Log form. The long events (>100=1 ms) were easily identified. c, the histogram of residual currents shows that there was a single peak current level of 17.4 pA when the ssDNA P1 interacts with the nanopore.

FIG. 17 shows melting temperature (Tm, ° C.) of the DNA C-C, mC-C and hmC-C with and without Ag⁺. a, The fluorescence curves (upper panel, −dI/dT vs T) and raw fluorescence curves (lower panel, fluorescence vs T) for C-C, mC-C and hmC-C mismatches. b, The fluorescence curves (upper panel) and raw fluorescence curves (lower panel) for C-Ag-C, mC-Ag-C and hmC-Ag-C mismatches. The data shown in upper panels were the inverse of the differential of the curve shown in the lower panels in each figure, i.e., −dI/dT. The peak positions represent the Tm value.

FIG. 18 shows that Ag⁺ doesn't interact with ssDNAs 1C, 1mC or 1hmC. a, The un-hybridized ssDNAs (when ssDNA 1C hybridized with P1) with and without Ag⁺ in the nanopore. Left panel: the histogram of the dwell time. Right panel: the histogram of residual currents (10-20 pA). b, The un-hybridized ssDNAs (when ssDNA 1mC hybridized with P1) with and without Ag⁺ in the nanopore. Left panel: the histogram of the dwell time. Right panel: the histogram of residual currents (10-20 pA). c, The un-hybridized ssDNAs (when ssDNA 1hmC hybridized with P1) with and without Ag⁺ in the nanopore. Left panel: the histogram of the dwell time. Right panel: the histogram of residual currents (10-20 pA). In a, b and c similar dwell times and residual currents can be identified. These values were very similar to that generated by ssDNA P1, which were 1.88 ms and 17.4 pA, respectively.

FIG. 19 shows that the addition of Ag⁺ decreased the residual current at different degrees for C-C and mC-C mismatches, but has no effect on hmC-C. C-C generated a peak of 42.1 pA, C-Ag-C generated a peak of 36.8 pA. The difference between C-C and C-Ag-C was 5.3 pA. mC-C generated a peak of 37.2 pA. mC-Ag-C generated two peaks of 33.9 pA and 38.1 pA. The difference was about 3.3 pA between mC-C and the first peak of mC-Ag-C. hmC-C generated a peak of 37.1 pA. hmC-Ag-C generated a similar peak of 36.2 pA.

FIG. 20 shows that the DNA duplex C-C (ssDNA 1C hybridized with P1) interacts with the nanopore at 180 mV. a, the histogram of residual currents. C-C generated a single peak of 50.5 pA; The C-Ag-C generated two peaks of 49.3 pA and 39.9 pA. The difference was about 10.6 pA between C-C and the second peak of C-Ag-C. b, the histogram of the dwell time in Log form. The C-C generated a single peak of 67 ms. The C-Ag-C generated two peaks of 49 ms and 151 ms.

Note that there are two residual current peaks for C-C with Ag⁺ at 180 mV, but only one peak at 150 mV (FIG. 12c2). The reason could be the DNA duplex dissociation was faster at 180 mV (49 ms and 151 ms) compared to 52 ms and 331 ms at 150 mV (FIG. 12c1). A 2.2 fold (331/151=2.2) decrease at 180 mV was observed. This shows a voltage-dependent dissociation. So C-Ag-C complexes could be dissociated too fast to sense the existence of the Ag⁺ sometimes at 180 mV, which correspond to the 49.3 pA residual current. Note that C-C has a similar residual current at 50.5 pA, which was very close to 49.3 pA.

FIG. 21 shows MD simulation of a DNA duplex with the C-C mismatch that is coordinated with a Ag⁺. a, Distances between the Ag⁺ and N3_{A} or between Ag⁺ and O2_{B}. In a binding state, these distances are about 2.06 Å. b, A snap-shot of a corresponding binding state from the simulation. c, Distances between the Ag⁺ and N3_{B} or between Ag⁺ and O2_{A} (blue). In a binding state, these distances are about 2.06 Å. d, A snap-shot of a corresponding binding state from the simulation. These results show that for a Ag⁺ there are two symmetric binding states (b and d) that are alternatively present in the simulated structure (a and c).

FIG. 22: shows probability densities of hydrogen-bond lengths between N3 and O2 atoms of difference bases in a mismatched pair. a, the mismatched pair is C-C. b, the mismatched pair is mC-C. The sharper peak in a indicates that the hydrogen-bond mediated base-pairing is more stable in the C-C mismatch.

The role of the hydroxyl group in the hmC (not shown): two examples of water mediated interaction between the phosphate group and the hydroxyl group in the hmC. The water molecule forms hydrogen bonds with both the phosphate group in the DNA backbone and the hydroxyl group in the hmC. Additionally, as shown in FIG. 14c, it is possible to form a direct interaction, via. the hydrogen bond, between the phosphate group and the hydroxyl group.

The key principle behind novel form of methylation determination is the fact that Ag+ interacts with and stabilizes a C-C containing DNA duplex. But the nature of coordination of Ag⁺ with C-C mismatches is not clearly understood. The alpha-hemolysin (α-HL) nanopore has a nanocavity (2.6 nm opening with a 1.4 nm constriction site) which can capture and hold the DNA duplex (FIG. 15) provides an ideal platform for studying the C-Ag-C interaction and how cytosine modifications change this interaction. The principle of a nanopore method is described in FIG. 15b. At first, it was tested how the ssDNA P1 (FIG. 11) interacts with the nanopore in KNO₃ solution. Short (>1 ms) and long events in the range of 1-10 ms were easily identified (FIG. 16). A similar result has been reported that KNO₃ has unknown effects on DNA translocation and some extraordinary long events were seen. In order to ensure the ssDNA interactions were excluded, we only considered events longer than 10 ms as the DNA duplex interaction in the following analysis.

At first, P2 (sequence in FIG. 11) was tried as the probe, because studies have found that when the probe was attached with an overhang, the capture rate can be greatly increased with a shorter unzipping time. When P2 was hybridized with 1C (sequence in FIG. 11), since there was a C-C mismatches in the duplex, the unzipping was very fast, the unzipping events were in the range of 0.5 ms-10 ms, which cannot be distinguished from the ssDNA P1. Since ssDNA itself can generate long events from 1 ms to 10 ms in KNO₃. So 10 ms was set as the cutoff point for DNA duplex capturing. But when P1 was used to hybridize with 1C, the dwell time was increased and can be separated from ssDNA.

In the nanopore recording, events longer than 10 ms were considered as the DNA duplex capturing. Identified were 50%-60% DNA duplexes trapping events (>10 ms) with an ending spike (FIG. 1, a1, a2, b1, b2), which was reported as unzipping signature in the nanopore S1. Two types of unzipping events can be observed, a1 and b1 (with two levels, large noise) vs b1 and b2 (with two levels, low noise). Similar phenomenon has been reported that DNA hairpins with a duplex blunt ending generate two main conductance states S2, S3.

Two residual current peaks were also observed for mC-C with Ag⁺ (33.9 pA and 38.1 pA, FIG. 13a4), but only a single peak for C-Ag-C (FIG. 12c2). This may be caused by the weak interaction between Ag⁺ and mC-C mismatches. Portion of the mCAg-C complexes could be easily dissociated just like without Ag⁺, which correspond to the 38.1 pA. Note that mC-C mismatches has a similar residual current at 37.2 pA (FIG. 13a4), which is very close to 38.1 pA.

The force field for Ag⁺ was adopted that was characterized for Ag⁺ in water. The force field for the interaction between Ag⁺ and a biomolecule is still not well developed. In MD simulation of Ag⁺ in a duplex with a C-C mismatch, the force field was adopted: εAg+/N3=0.218 kcal/mol; εAg+/O2=0.169 kcal/mol; σAg+/N3=0.227 nm; σAg+/O2=0.227 nm. As shown in FIG. 22, the mean distance between Ag⁺ and a N3 atom in a binding state is about 0.206 nm, consistent with the distance found in the crystal structure (PDB: 2KE8). The key principle behind novel form of methylation determination is the fact that Ag⁺ interacts with and stabilizes a C-C containing DNA duplex. But the nature of coordination of Ag⁺ with C-C mismatches is not clearly understood. The alpha-hemolysin (α-HL) nanopore has a nanocavity (2.6 nm opening with a 1.4 nm constriction site) which can capture and hold the DNA duplex (FIG. 15a) provides an ideal platform for studying the C-Ag-C interaction and how cytosine modifications change this interaction. The principle of a nanopore method is described in FIG. 15b. At first, it was tested how the ssDNA P1 (FIG. 11) interacts with the nanopore in KNO₃ solution. Short (>1 ms) and long events in the range of 1-10 ms were easily identified (FIG. 16). A similar result has been reported that KNO₃ has unknown effects on DNA translocation and some extraordinary long events were seen. In order to ensure the ssDNA interactions were excluded, it was only considered events longer than 10 ms as the DNA duplex interaction.

### Example 3

Cancers arise as a result of accumulation of changes in the DNA in cancer cells. Even though, it doesn't means all of the mutations are involved in cancer development. Driver mutation plays important role in oncogenesis. It has conferred growth advantage on the cancer cell and has been positively selected in the microenvironment of the tissue where the cancer arises. Oppositely, a passenger mutation has not been selected, has not conferred clonal growth advantage and has therefore not contributed to cancer development. Passenger mutations are found within cancer genomes because somatic mutations without functional consequences often occur during cell division. Thus, a cell that acquires a driver mutation will already have biologically inert somatic mutations within its genome.

Serine/threonine-protein kinase B-raf (BRAF), a member of the Raf family, is encoded by gene BRAF. BRAF mutations are frequent in benign and malignant human tumors. BRAF V600E, a driver mutation accounts for the vast majority of BRAF alterations and the mutation induces a conformational change of the activation segment leading to a constitutive kinase activity of BRAF and consecutive phosphorylation of downstream targets. BRAF V600E mutation have been detected in melanoma, pleomorphic xanthoastrocytomas, papillary thyroid carcinoma, and some other kinds of cancers. Moreover, this driver mutation has been involved in the table of phamacogenomic biomarkers in drug lables in FDA website.

Genetic coden changes from “GTG” to “GAG” in BRAF V600E mutation. Mercuric ion (Hg²⁺) binds with the T-T mismatched base pair to generate a novel metal-mediated base pair in duplex DNA. And the melting temperature can be enhanced significantly According to previously obtained results, a nucleic acid duplex with overhangs can detected in nanopore easily. Here, nanopore platform was used to detect BRAF V600E mutation. A series of DNA probes were designed and synthesized. Hg²⁺ was added to single-stranded target and probe DNA. Oligonucleotides were denatured at 94° C. and cooled at room temperature. The Hg²⁺ bound duplex generated signature facilitates discrimination of the mutation in the gene.

Probes were designed to detect the mutations on both sense and anti-sense strands of the

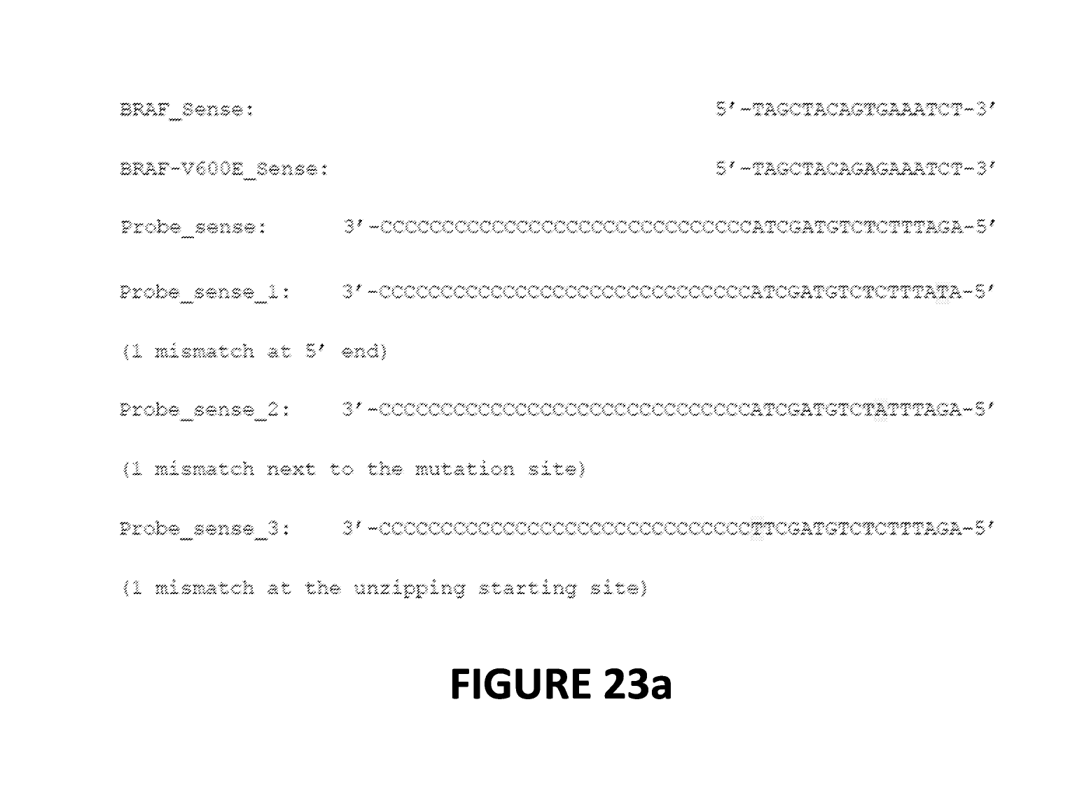

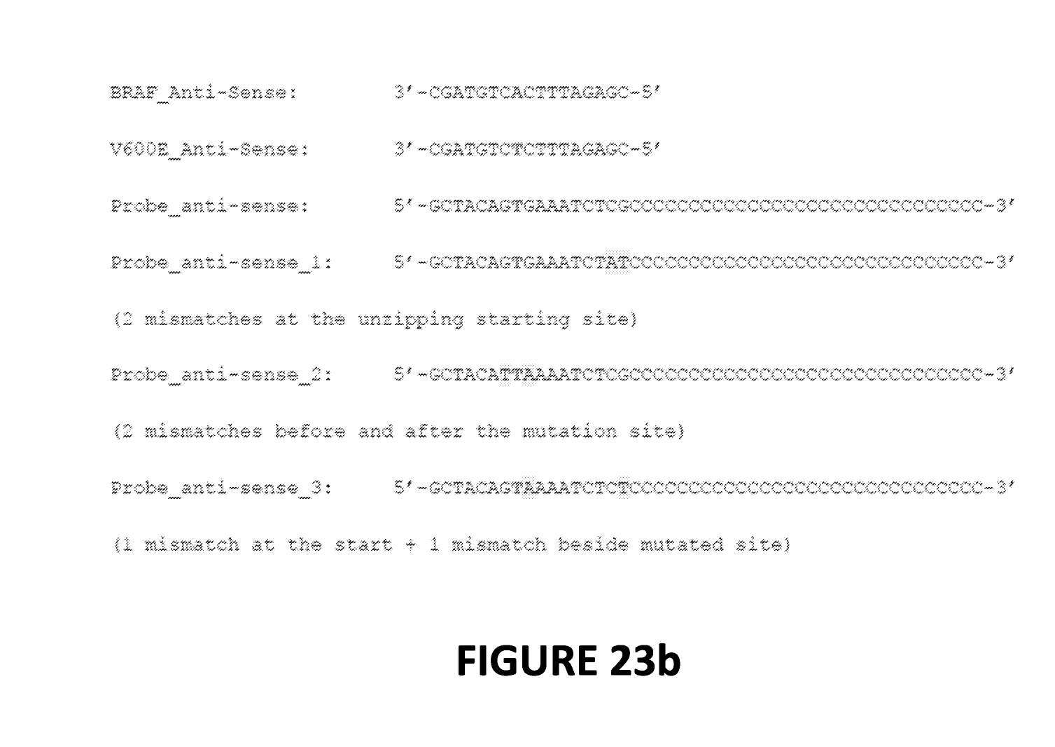

BRAF gene (FIG. 23a,b for sequences).

Nanopore will be used to determine the target:probe complex unzipping time in the nanopore. In the presence of Hg²⁺, if the unzipping time is short in the millisecond scale, it would indicate there is no inter-strand lock formation, and the DNA:probe hybridization is weak. This would suggest that the tested nucleotide is an adenine, but not thymine. Contrarily, if the unzipping time in the nanopore is increased by 2 orders of magnitude to the scale of ˜100 milliseconds, this indicates a strong inter-strand lock is formed. This result would suggest that the tested nucleotide is a thymine, but not adenine. The schedule is given in the Table 5. To date, the anti-sense strand with mutation has been tested and verified.

TABLE 5

| | | Without | Hybrid- | | |
|---|---|---|---|---|---|
| Sense/ | Normal/ | (−)/with | ization | unzipping | |
| Anti-sense | mutant | (+) | strength- | time in | To be tested/ |
| strand | gene | Hg2+ | ening | nanopore | verified |
| Sense | Normal | − | − | ~1 ms | To be tested |
| strand | (−GTG−) | | | | |
| | | + | + | ~100 ms | To be tested |
| Sense | Mutant | − | − | ~1 ms | To be tested |
| strand | (−GAG−) | | | | |
| | | + | − | ~1 ms | To be tested |
| Anti-sense | Normal | − | − | ~1 ms | To be tested |
| strand | (−CAC−) | | | | |
| | | + | − | ~1 ms | To be tested |
| Anti-sense | Mutant | − | − | ~2 ms | Verified |
| strand | (−CTC−) | | | | |
| | | + | + | ~100 ms | Verified |

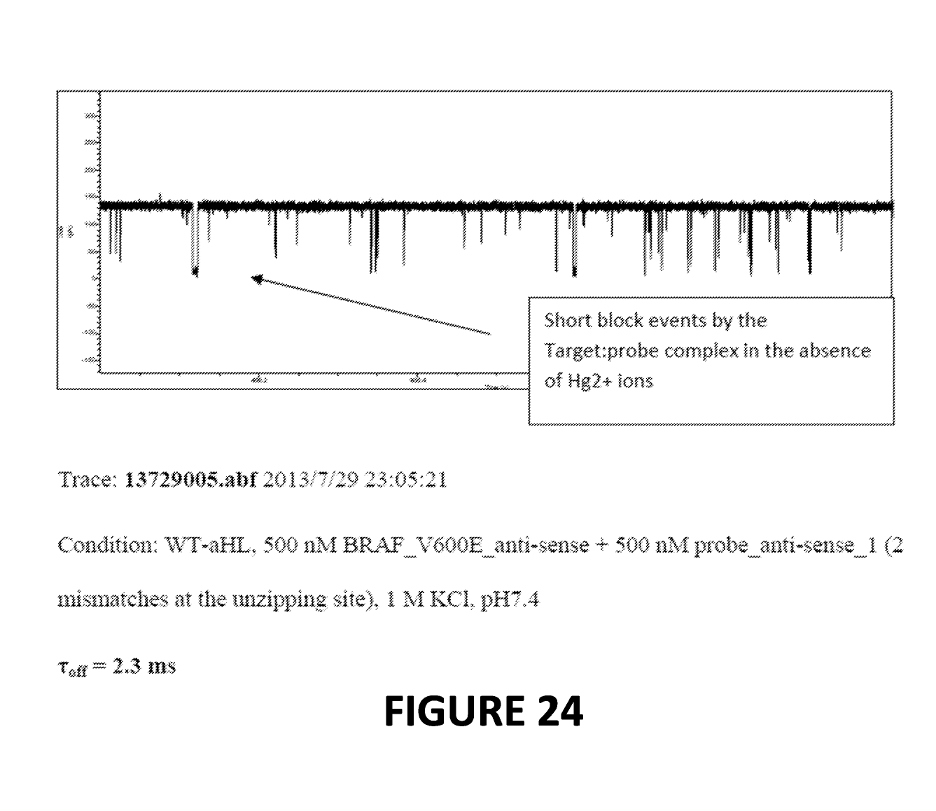

FIG. 24 shows the BRAF-V600E mutant gene, anti-sense strand, and detection using Probe_anti-sense_1. In the absence of Hg²⁺, short block events were observed for the target:probe complex that unzipping quickly in the nanopore. The unzipping time was 2.3 ms. No T-Hg-T inter-strand lock can be formed.

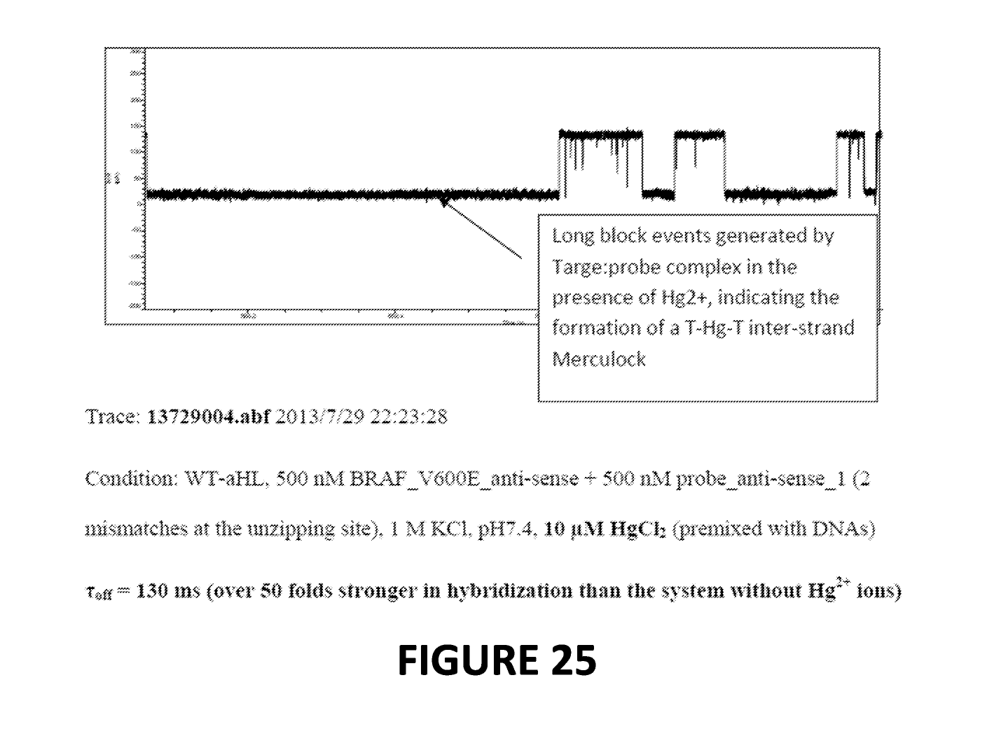

FIG. 25 shows the BRAF-V600E mutant gene, anti-sense strand, and detection using Probe_anti-sense_1. In the presence of Hg²⁺, long block events were observed for the target:probe complex that take longer time to unzip in the nanopore. The unzipping time was 130 ms, a 2 orders of magnitude increase compared with the case in the absence of Hg²⁺. There formed a strong T-Hg-T inter-strand lock.

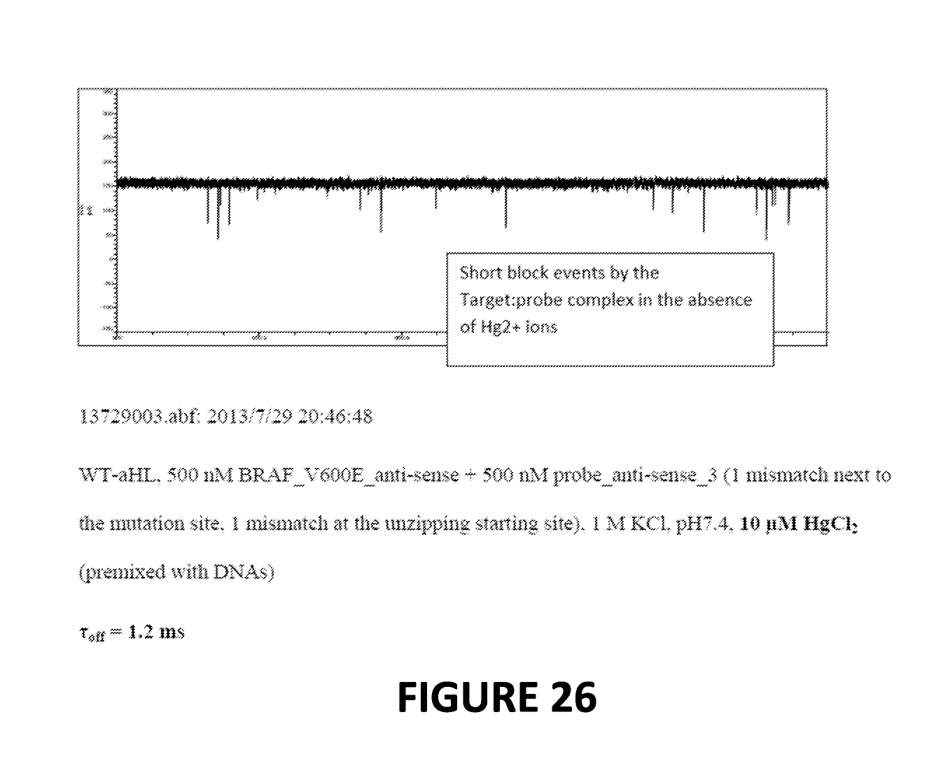

FIG. 26 shows the BRAF-V600E mutant gene, anti-sense strand, and detection using Probe_anti-sense_2. In the absence of Hg²⁺, short block events were observed for the target:probe complex that unzipping quickly in the nanopore. The unzipping time was 1.2 ms. No T-Hg-T inter-strand lock can be formed.

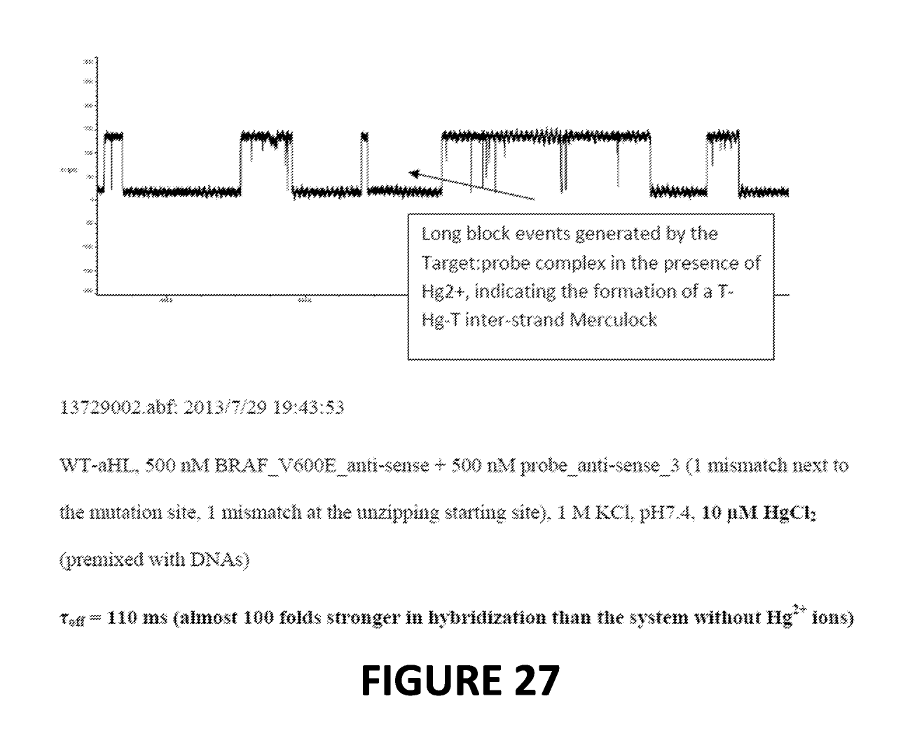

FIG. 27 shows the BRAF-V600E mutant gene, anti-sense strand, and detection using Probe_anti-sense_2. In the presence of Hg²⁺, long block events were observed for the target:probe complex that take longer time to unzip in the nanopore. The unzipping time was 130 ms, a 2 orders of magnitude increase compared with the case in the absence of Hg²⁺. There formed a strong T-Hg-T inter-strand lock.

The foregoing description of the specific embodiments will so fully reveal the general nature of the provided embodiments that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

### REFERENCES

1. Suzuki, M. M. & Bird, A. DNA methylation landscapes: Provocative insights from epigenomics. Nat. Rev. Gen. 9, 465-476 (2008).
2. Deaton, A. M. & Bird, A. CpG islands and the regulation of transcription. Genes Dev.
25, 1010-1022 (2011).
3. Lim, P. S., Shannon, M. F., & Hardy, K. Epigenetic control of inducible gene expression in the immune system. Epigenomics 2, 775-795 (2010).
4. Jones, P. A. & Baylin, S. B. The fundamental role of epigenetic events in cancer. Nat. Rev. Gen. 3, 415-428 (2002).
5. Bannister, A. J. & Kouzarides, T. Regulation of chromatin by histone modifications. Cell Res. 21, 381-395 (2011).
6. Egger, G., Liang, G., Aparicio, A., & Jones, P. A. Epigenetics in human disease and prospects for epigenetic therapy. Nature 429, 457-463 (2004).
7. Beck, S. & Rakyan, V. K. The methylome: approaches for global DNA methylation profiling. Trends Genet. 24, 231-237 (2008).
8. Bestor, T. H. The DNA methyltransferases of mammals. Hum. Mol. Genet. 9, 2395-2402 (2000).
9. Bird, A. The essentials of DNA methylation. Cell 70, 5-8 (1992).
10. Craig, J. M. & Bickmore, W. A. The distribution of CpG islands in mammalian chromosomes. NAT GENET. 7, 376-382 (1994).
11. Illingworth, R. S. & Bird, A. P. CpG islands—‘A rough guide’. FEBS Lett. 583, 1713-1720 (2009).
12. Bernstein, B. E., Meissner, A., & Lander, E. S. The mammalian epigenome. Cell 128, 669-681 (2007).
13. Baylin, S. B. & Jones, P. A. A decade of exploring the cancer epigenome-biological and translational implications. Nat. Rev. Cancer 11, 726-734 (2011).
14. Esteller, M. Molecular origins of cancer: Epigenetics in cancer. New Engl. J. Med. 358, 1148-1159+1096 (2008).
15. Basu, R. & Zhang, L. F. X chromosome inactivation: A silence that needs to be broken. Genesis 49, 821-834 (2011).
16. Baylin, S. B. & Ohm, J. E. Epigenetic gene silencing in cancer—A mechanism for early oncogenic pathway addiction? Nat. Rev. Cancer 6, 107-116 (2006).
17. Shi, H., Wang, M. X., & Caldwell, C. W. CpG islands: Their potential as biomarkers for cancer. Expert Rev. Mol. Diagn. 7, 519-531 (2007).
18. Esteller, M. Relevance of DNA methylation in the management of cancer. Lancet Oncol. 4, 351-358 (2003).
19. GrØnbek, K., Hother, C., & Jones, P. A. Epigenetic changes in cancer. APMIS 115, 1039-1059 (2007).
20. Holloway, A. F. & Oakford, P. C. Targeting epigenetic modifiers in cancer. Curr. Med. Chem. 14, 2540-2547 (2007).
21. Issa, J. P. J. DNA methylation as a therapeutic target in cancer. Clin. Cancer Res. 13, 1634-1637 (2007).
22. Shivapurkar, N. & Gazdar, A. F. DNA methylation based biomarkers in non-invasive cancer screening. Curr. Mol. Med. 10, 123-132 (2010).
23. Reinders, J. & Paszkowski, J. Bisulfite methylation profiling of large genomes. Epigenomics. 2, 209-220 (2010).
24. Harris, R. A., Wang, T., Coarfa, C., Nagarajan, R. P., Hong, C., Downey, S. L., Johnson, B. E., Fouse, S. D., Delaney, A., Zhao, Y., Olshen, A., Ballinger, T., Zhou, X., Forsberg, K. J., Gu, J., Echipare, L., O'Geen, H., Lister, R., Pelizzola, M., Xi, Y., Epstein, C. B., Bernstein, B. E., Hawkins, R. D., Ren, B., Chung, W. Y., Gu, H., Bock, C., Gnirke, A., Zhang, M. Q., Haussler, D., Ecker, J. R., Li, W., Farnham, P. J., Waterland, R. A., Meissner, A., Marra, M. A., Hirst, M., Milosavljevic, A., & Costello, J. F. Comparison of sequencing-based methods to profile DNA methylation and identification of monoallelic epigenetic modifications. Nat. Biotechnol. 28, 1097-1105 (2010).
25. Adorjàn, P., Distler, J., Lipscher, E., Model, F., Müller, J., Pelet, C., Braun, A., Florl, A. R., Gütig, D., Grabs, G., Howe, A., Kursar, M., Lesche, R., Leu, E., Lewin, A., Maier, S., Müller, V., Otto, T., Scholz, C., Schulz, W. A., Seifert, H. H., Schwope, I., Ziebarth, H., Berlin, K., Piepenbrock, C., & Olek, A. Tumour class prediction and discovery by microarray-based DNA methylation analysis. Nucleic Acids Res 30, (2002).
26. Herman, J. G., Graff, J. R., My+ [symbol] h+ñnen, S., Nelkin, B. D., & Baylin, S. B. Methylationspecific PCR: A novel PCR assay for methylation status of CpG islands. PROC. NATL. ACAD. SCI. U.S.A 93, 9821-9826 (1996).
27. Eads, C. A., Danenberg, K. D., Kawakami, K., Saltz, L. B., Blake, C., Shibata, D., Danenberg, P. V., & Laird, P. W. MethyLight: a high-throughput assay to measure DNA methylation. Nucleic Acids Res 28, (2000).
28. Ehrich, M., Nelson, M. R., Stanssens, P., Zabeau, M., Liloglou, T., Xinarianos, G., Cantor, C. R., Field, J. K., & Van Den Boom, D. Quantitative high-throughput analysis of DNA methylation patterns by base-specific cleavage and mass spectrometry. PROC. NATL. ACAD. SCI. U.S.A 102, 15785-15790 (2005).
29. Fouse, S. D., Nagarajan, R. P., & Costello, J. F. Genome-scale DNA methylation analysis. Epigenomics 2, 105-117 (2010).
30. Eads, C. A. & Laird, P. W. Combined bisulfite restriction analysis (COBRA). Methods Mol. Biol. 200, 71-85 (2002).
31. Clark, S. J., Statham, A., Stirzaker, C., Molloy, P. L., & Frommer, M. DNA methylation: Bisulphite modification and analysis. Nat. Protoc. 1, 2353-2364 (2006).
32. Song, C. X., Clark, T. A., Lu, X. Y., Kislyuk, A., Dai, Q., Turner, S. W., He, C., & Korlach, J. Sensitive and specific single-molecule sequencing of 5-hydroxymethylcytosine. Nat. Methods 9, 75-77 (2012).
33. Zhu, R., Howorka, S., Proll, J., Kienberger, F., Preiner, J., Hesse, J., Ebner, A., Pastushenko, V. P., Gruber, H. J., & Hinterdorfer, P. Nanomechanical recognition measurements of individual DNA molecules reveal epigenetic methylation patterns. Nat. Nanotechnol. 5, 788-791 (2010).
34. Dedeurwaerder, S., Defrance, M., Calonne, E., Denis, H., Sotiriou, C., & Fuks, F. Evaluation of the Infinium Methylation 450K technology. Epigenomics. 3, 771-784 (2011).
35. Bayley, H. & Cremer, P. S. Stochastic sensors inspired by biology. Nature 413, 226-230 (2001).
36. Bayley, H., Cronin, B., Heron, A., Holden, M. A., Hwang, W. L., Syeda, R., Thompson, J., & Wallace, M. Droplet interface bilayers. Mol. Biosyst. 4, 1191-1208 (2008).
37. Gu, L. Q. & Shim, J. W. Single molecule sensing by nanopores and nanopore devices. Analyst 135, 441-451 (2010).
38. Hornblower, B., Coombs, A., Whitaker, R. D., Kolomeisky, A., Picone, S. J., Meller, A., & Akeson, M. Single-molecule analysis of DNA-protein complexes using nanopores. Nat Methods 4, 315-317 (2007).
39. Howorka, S. & Siwy, Z. Nanopore analytics: Sensing of single molecules. Chem. Soc. Rev. 38, 2360-2384 (2009).
40. Movileanu, L. Interrogating single proteins through nanopores: challenges and opportunities. Trends Biotechnol. 27, 333-341 (2009).
41. Ma, L. & Cockroft, S. L. Biological nanopores for single-molecule biophysics. Chembiochem 11, 25-34 (2010).
42. Majd, S., Yusko, E. C., Billeh, Y. N., Macrae, M. X., Yang, J., & Mayer, M. Applications of biological pores in nanomedicine, sensing, and nanoelectronics. Current Opinion in Biotechnology 21, 439-476 (2010).
43. Olasagasti, F., Lieberman, K. R., Benner, S., Cherf, G. M., Dahl, J. M., Deamer, D. W., & Akeson, M. Replication of individual DNA molecules under electronic control using a protein nanopore. Nat Nanotechnol. 5, 798-806 (2010).
44. Venkatesan, B. M. & Bashir, R. Nanopore sensors for nucleic acid analysis. Nat Nanotechnol. 6, 615-624 (2011).
45. Wanunu, M., Morrison, W., Rabin, Y., Grosberg, A. Y., & Meller, A. Electrostatic focusing of unlabelled DNA into nanoscale pores using a salt gradient. Nat. Nanotechnol. 5, 160-165 (2010).
46. Wanunu, M., Dadosh, T., Ray, V., Jin, J., McReynolds, L., & Drndi-ç, M. Rapid electronic detection of probe-specific microRNAs using thin nanopore sensors. Nat. Nanotechnol. 5, 807-814 (2010).
47. Wendell, D., Jing, P., Geng, J., Subramaniam, V., Lee, T. J., Montemagno, C., & Guo, P. Translocation of double-stranded DNA through membrane-adapted phi29 motor protein nanopores. Nat Nanotechnol. 4, 765-772 (2009).
48. Purnell, R. F. & Schmidt, J. J. Discrimination of single base substitutions in a DNA strand immobilized in a biological nanopore. ACS Nano 3, 2533-2538 (2009).
49. Chu, J., Gonzalez-Lopez, M., Cockroft, S. L., Amorin, M., & Ghadiri, M. R. Real-time monitoring of DNA polymerase function and stepwise single-nucleotide DNA strand translocation through a protein nanopore. Angew. Chem. Int. Ed Engl. 49, 10106-10109 (2010).
50. Cherf, G. M., Lieberman, K. R., Rashid, H., Lam, C. E., Karplus, K., & Akeson, M. Automated forward and reverse ratcheting of DNA in a nanopore at 5-A precision. Nat Biotechnol 30, 344-348 (2012).
51. Manrao, E. A., Derrington, I. M., Laszlo, A. H., Langford, K. W., Hopper, M. K., Gillgren, N., Pavlenok, M., Niederweis, M., & Gundlach, J. H. Reading DNA at single-nucleotide resolution with a mutant MspA nanopore and phi29 DNA polymerase. Nat Biotechnol 30, 349-353 (2012).
52. Clarke, J., Wu, H. C., Jayasinghe, L., Patel, A., Reid, S., & Bayley, H. Continuous base identification for single-molecule nanopore DNA sequencing. Nat. Nanotechnol. 4, 265-270 (2009).
53. Hall, A. R., Scott, A., Rotem, D., Mehta, K. K., Bayley, H., & Dekker, C. Hybrid pore formation by directed insertion of alpha-haemolysin into solid-state nanopores. Nat Nanotechnol. 5, 874-877 (2010).
54. Branton, D., Deamer, D. W., Marziali, A., Bayley, H., Benner, S. A., Butler, T., Di Ventra, M., Garaj, S., Hibbs, A., Huang, X., Jovanovich, S. B., Krstic, P. S., Lindsay, S., Ling, X. S., Mastrangelo, C. H., Meller, A., Oliver, J. S., Pershin, Y. V., Ramsey, J. M., Riehn, R., Soni, G. V., Tabard-Cossa, V., Wanunu, M., Wiggin, M., & Schloss, J. A. The potential and challenges of nanopore sequencing. Nature Biotechnology 26, 1146-1153 (2008).
55. Kasianowicz, J. J., Brandin, E., Branton, D., & Deamer, D. W. Characterization of individual polynucleotide molecules using a membrane channel. PROC. NATL. ACAD. SCI. U.S.A 93, 13770-13773 (1996).
56. An, N., Fleming, A. M., White, H. S., & Burrows, C. J. Crown ether-electrolyte interactions permit nanopore detection of individual DNA abasic sites in single molecules. Proc Natl. Acad. Sci. U. S. A 109, 11504-11509 (2012).
57. Wang, Y., Zheng, D., Tan, Q., Wang, M. X., & Gu, L. Q. Nanopore-based detection of circulating microRNAs in lung cancer patients. Nat. Nanotechnol. 6, 668-674 (2011).
58. Wallace, E. V. B., Stoddart, D., Heron, A. J., Mikhailova, E., Maglia, G., Donohoe, T. J., & Bayley, H. Identification of epigenetic DNA modifications with a protein nanopore. Chem. Commun. 46, 8195-8197 (2010).
59. Manrao, E. A., Derrington, I. M., Pavlenok, M., Niederweis, M., & Gundlach, J. H. Nucleotide discrimination with DNA immobilized in the MspA nanopore. PLoS. One. 6, e25723 (2011).
60. Shim, J., Humphreys, G. I., Venkatesan, B. M., Munz, J. M., Zou, X., Sathe, C., Schulten, K., Kosari, F., Nardulli, A. M., Vasmatzis, G., & Bashir, R. Detection and quantification of methylation in DNA using solid-state nanopores. Sci. Rep. 3, 1389 (2013).
61. Mirsaidov, U., Timp, W., Zou, X., Dimitrov, V., Schulten, K., Feinberg, A. P., & Timp, G. Nanoelectromechanics of methylated DNA in a synthetic nanopore. Biophys. J. 96, L32-L34 (2009).
62. Wanunu, M., Cohen-Karni, D., Johnson, R. R., Fields, L., Benner, J., Peterman, N., Zheng, Y., Klein, M. L., & Drndic, M. Discrimination of methylcytosine from hydroxymethylcytosine in DNA molecules. J. Am. Chem. Soc. 133, 486-492 (2011).
63. Clever, G. H., Kaul, C., & Carell, T. DNA—metal base pairs. Angew. Chem Int. Ed Engl. 46, 6226-6236 (2007).
64. Interplay Between Metal ions and Nucleic Acids (Springer, 2012).
65. Muller, J. Functional metal ions in nucleic acids. Metallomics. 2, 318-327 (2010).
66. Huang, K. & Mart+ [symbol] , A. A. Recent trends in molecular beacon design and applications. Analytical and Bioanalytical Chemistry 402, 3091-3102 (2012).
67. Miyake, Y., Togashi, H., Tashiro, M., Yamaguchi, H., Oda, S., Kudo, M., Tanaka, Y., Kondo, Y., Sawa, R., Fujimoto, T., Machinami, T., & Ono, A. Mercury II-mediated formation of thymine-Hg II-thymine base pairs in DNA duplexes. J. Am. Chem. Soc. 128, 2172-2173 (2006).
68. Nolan, E. M. & Lippard, S. J. Tools and tactics for the optical detection of mercuric ion. Chem. Rev. 108, 3443-3480 (2008).
69. Ono, A. & Togashi, H. Highly selective oligonucleotide-based sensor for mercury(II) in aqueous solutions. Angew. Chem. Int. Ed. 43, 4300-4302 (2004).
70. Ono, A., Torigoe, H., Tanaka, Y., & Okamoto, I. Binding of metal ions by pyrimidine base pairs in DNA duplexes. Chem. Soc. Rev. 40, 5855-5866 (2011).
71. Selid, P. D., Xu, H., Collins, E. M., Face-Collins, M. S., & Zhao, J. X. Sensing mercury for biomedical and environmental monitoring. Sensors 9, 5446-5459 (2009).
72. Tanaka, Y., Oda, S., Yamaguchi, H., Kondo, Y., Kojima, C., & Ono, A. 15N-15N Jcoupling across Hg II: Direct observation of Hg II-mediated T-T base pairs in a DNA duplex. J. Am. Chem. Soc. 129, 244-245 (2007).
73. Wen, S., Zeng, T., Liu, L., Zhao, K., Zhao, Y., Liu, X., & Wu, H. C. Highly sensitive and selective DNA-based detection of mercury(II) with alpha-hemolysin nanopore. J. Am. Chem. Soc. 133, 18312-18317 (2011).
74. Wang, G., Zhao, Q., Kang, X., & Guan, X. Probing mercury(II)-DNA interactions by nanopore stochastic sensing. J. Phys. Chem. B 117, 4763-4769 (2013).
75. Miyake, Y., Togashi, H., Tashiro, M., Yamaguchi, H., Oda, S., Kudo, M., Tanaka, Y., Kondo, Y., Sawa, R., Fujimoto, T., Machinami, T., & Ono, A. MercuryII-mediated formation of thymine-HgII-thymine base pairs in DNA duplexes. J Am. Chem Soc 128, 2172-2173 (2006).
76. Belinsky, S. A., Liechty, K. C., Gentry, F. D., Wolf, H. J., Rogers, J., Vu, K., Haney, J., Kennedy, T. C., Hirsch, F. R., Miller, Y., Franklin, W. A., Herman, J. G., Baylin, S. B., Bunn, P. A., & Byers, T. Promoter hypermethylation of multiple genes in sputum precedes lung cancer incidence in a high-risk cohort. Cancer Res. 66, 3338-3344 (2006).
77. Esteller, M., Corn, P. G., Baylin, S. B., & Herman, J. G. A gene hypermethylation profile of human cancer. Cancer Res. 61, 3225-3229 (2001).
78. Marsit, C. J., Houseman, E. A., Christensen, B. C., Eddy, K., Bueno, R., Sugarbaker, D. J., Nelson, H. H., Karagas, M. R., & Kelsey, K. T. Examination of a CpG island methylator phenotype and implications of methylation profiles in solid tumors. Cancer Res. 66, 10621-10629 (2006).
79. Wang, Y. C., Hsu, H. S., Chen, T. P., & Chen, J. T. Molecular diagnostic markers for lung cancer in sputum and plasma. Ann. N. Y. Acad. Sci. 1075, 179-184 (2006).
80. Baaken, G., Sondermann, M., Schlemmer, C., Ruhe, J., & Behrends, J. C. Planar microelectrode-cavity array for high-resolution and parallel electrical recording of membrane ionic currents. Lab Chip 8, 938-944 (2008).
81. Baaken, G., Ankri, N., Schuler, A. K., Ruhe, J., & Behrends, J. C. Nanopore-based single molecule mass spectrometry on a lipid membrane microarray. ACS Nano 5, 8080-8088 (2011).
82. Krueger, F., Kreck, B., Franke, A., & Andrews, S. R. DNA methylome analysis using short bisulfite sequencing data. Nat. Methods 9, 145-151 (2012).
83. Sepulveda, A. R., Jones, D., Ogino, S., Samowitz, W., Gulley, M. L., Edwards, R., Levenson, V., Pratt, V. M., Yang, B., Nafa, K., Yan, L., & Vitazka, P. CpG methylation analysis—Current status of clinical assays and potential applications in molecular diagnostics: A report of the association for molecular pathology. J. Mol. Diagn. 11, 266-278 (2009).
84. Shim, J. W., Tan, Q., & Gu, L. Q. Single-molecule detection of folding and unfolding of a single G-quadruplex aptamer in a nanopore nanocavity. Nucleic Acids Res 37, 972-982 (2009).
85. Lister, R. et al. Human DNA methylomes at base resolution show widespread epigenomic differences. Nature 462, 315-322 (2009).
86. Brena, R. M., Huang, T. H. & Plass, C. Quantitative assessment of DNA methylation: potential applications for disease diagnosis, classification, and prognosis in clinical settings. Journal of molecular medicine 84, 365-377 (2006).
87. Kriaucionis, S. & Heintz, N. The nuclear DNA base 5-hydroxymethylcytosine is present in Purkinje neurons and the brain. Science 324, 929-930 (2009).
88. Nomura, A., Sugizaki, K., Yanagisawa, H. & Okamoto, A. Discrimination between 5-hydroxymethylcytosine and 5-methylcytosine by a chemically designed peptide. Chemical communications 47, 8277-8279 (2011).
89. Szwagierczak, A. et al. Characterization of PvuRts1I endonuclease as a tool to investigate genomic 5-hydroxymethylcytosine. Nucleic acids research 39, 5149-5156 (2011).
90. Flusberg, B. A. et al. Direct detection of DNA methylation during single-molecule, real-time sequencing. Nature methods 7, 461-465 (2010).
91. Ito, S. et al. Role of Tet proteins in 5mC to 5hmC conversion, ES-cell self-renewal and inner cell mass specification. Nature 466, 1129-1133 (2010).
92. Ono, A. et al. Specific interactions between silver(I) ions and cytosine-cytosine pairs in DNA duplexes. Chemical communications, 4825-4827 (2008).
93. Lin, Y. H. & Tseng, W. L. Highly sensitive and selective detection of silver ions and silver nanoparticles in aqueous solution using an oligonucleotide-based fluorogenic probe. Chemical communications, 6619-6621 (2009).
94. Wen, Y. et al. A graphene-based fluorescent nanoprobe for silver(I) ions detection by using graphene oxide and a silver-specific oligonucleotide. Chemical communications 46, 2596-2598 (2010).
95. Torigoe, H. et al. Thermodynamic and structural properties of the specific binding between Ag(+) ion and C:C mismatched base pair in duplex DNA to form C-Ag-C metal-mediated base pair. Biochimie 94, 2431-2440 (2012).
96. Torigoe, H., Miyakawa, Y., Ono, A. & Kozasa, T. Thermodynamic properties of the specific binding between Ag+ ions and C:C mismatched base pairs in duplex DNA. Nucleosides, nucleotides & nucleic acids 30, 149-167 (2011).
97. Kistenmacher, T. J., Rossi, M. & Marzilli, L. G. Crystal and molecular structure of (nitrato)(1-methylcytosine) silver(I): An unusual cross-linked polymer containing a heavy metal and a modified nucleic acid constituent. Inorganic Chemistry 18, 240-244 (1979).
98. Wen, S. et al. Highly sensitive and selective DNA-based detection of mercury(II) with alpha-hemolysin nanopore. Journal of the American Chemical Society 133, 18312-18317 (2011).
99. Wang, Y., Zheng, D., Tan, Q., Wang, M. X. & Gu, L. Q. Nanopore-based detection of circulating microRNAs in lung cancer patients. Nature nanotechnology 6, 668-674 (2011).
100. Nightingale Jr, E. R. Phenomenological theory of ion solvation. Effective radii of hydrated ions. Journal of Physical Chemistry 63, 1381-1387 (1959).
101. Wanunu, M. et al. Discrimination of methylcytosine from hydroxymethylcytosine in DNA molecules. Journal of the American Chemical Society 133, 486-492 (2011).
102. Gunther L, E. et al. in Bioinorganic Chemistry, Vol. 100 135-154 (AMERICAN CHEMICAL SOCIETY, 1971).
103. Wallace, E. V. et al. Identification of epigenetic DNA modifications with a protein nanopore. Chemical communications 46, 8195-8197 (2010).
104. Li, W. W., Gong, L. & Bayley, H. Single-molecule detection of 5-hydroxymethylcytosine in DNA through chemical modification and nanopore analysis. Angewandte Chemie 52, 4350-4355 (2013).
105. Shim, J. et al. Detection and quantification of methylation in DNA using solid-state nanopores. Scientific reports 3 (2013).
106. Atwell, S., Meggers, E., Spraggon, G. & Schultz, P. G. Structure of a copper-mediated base pair in DNA. Journal of the American Chemical Society 123, 12364-12367 (2001).
107. Okamoto, I., Iwamoto, K., Watanabe, Y., Miyake, Y. & Ono, A. Metal-ion selectivity of chemically modified uracil pairs in DNA duplexes. Angewandte Chemie 48, 1648-1651 (2009).
108. Shim, J. W., Tan, Q. & Gu, L. Q. Single-molecule detection of folding and unfolding of the G-quadruplex aptamer in a nanopore nanocavity. Nucleic acids research 37, 972-982 (2009).
109. Phillips, J. C. et al. Scalable molecular dynamics with NAMD. Journal of computational chemistry 26, 1781-1802 (2005).
110. MacKerell Jr, A. D. et al. All-atom empirical potential for molecular modeling and dynamics studies of proteins. Journal of Physical Chemistry B 102, 3586-3616 (1998).
111. Jorgensen, W. L., Chandrasekhar, J., Madura, J. D., Impey, R. W. & Klein, M. L. Comparison of simple potential functions for simulating liquid water. The Journal of Chemical Physics 79, 926-935 (1983).
112. Beglov, D. & Roux, B. Finite representation of an infinite bulk system: Solvent boundary potential for computer simulations. The Journal of Chemical Physics 100, 9050-9063 (1994).
113. D. Dankort, D. P. Curley, R. A. Cartlidge, B. Nelson, A. N. Karnezis, W. E. Damsky, Jr., M. J. You, R. A. DePinho, M. McMahon, and M. Bosenberg, ‘Braf(V600e) Cooperates with Pten Loss to Induce Metastatic Melanoma’, Nat Genet, 41 (2009), 544-52.
114. D. Dias-Santagata, Q. Lam, K. Vernovsky, N. Vena, J. K. Lennerz, D. R. Borger, T. T. Batchelor, K. L. Ligon, A. J. Iafrate, A. H. Ligon, D. N. Louis, and S. Santagata, ‘Braf V600e Mutations Are Common in Pleomorphic Xanthoastrocytoma: Diagnostic and Therapeutic Implications’, PLoS One, 6 (2011), e17948.
115. I. V. Fedorenko, K. H. Paraiso, and K. S. Smalley, ‘Acquired and Intrinsic Braf Inhibitor Resistance in Braf V600e Mutant Melanoma’, Biochem Pharmacol, 82 (2011), 201-9.
116. F. Frasca, C. Nucera, G. Pellegriti, P. Gangemi, M. Attard, M. Stella, M. Loda, V. Vella, C. Giordano, F. Trimarchi, E. Mazzon, A. Belfiore, and R. Vigneri, ‘Braf(V600e) Mutation and the Biology of Papillary Thyroid Cancer’, Endocr Relat Cancer, 15 (2008), 191-205.
117. P. Hou, D. Liu, J. Dong, and M. Xing, ‘The Braf(V600e) Causes Widespread Alterations in Gene Methylation in the Genome of Melanoma Cells’, Cell Cycle, 11 (2012), 286-95.
118. S. M. Kumar, H. Yu, R. Edwards, L. Chen, S. Kazianis, P. Brafford, G. Acs, M. Herlyn, and X. Xu, ‘Mutant V600e Braf Increases Hypoxia Inducible Factor-1 alpha Expression in Melanoma’, Cancer Res, 67 (2007), 3177-84.
119. J. Park, ‘Melanoma-Targeted Therapy Based on V600e Braf Mutation’, Biomark Med, 4 (2010), 792.
120. S. Y. Park, Y. J. Park, Y. J. Lee, H. S. Lee, S. H. Choi, G. Choe, H. C. Jang, S. H. Park, J. Park do, and B. Y. Cho, ‘Analysis of Differential Braf(V600e) Mutational Status in Multifocal Papillary Thyroid Carcinoma: Evidence of Independent Clonal Origin in Distinct Tumor Foci’, Cancer, 107 (2006), 1831-8.
121. L. R. Rowe, B. G. Bentz, and J. S. Bentz, ‘Detection of Braf V600e Activating Mutation in Papillary Thyroid Carcinoma Using Pcr with Allele-Specific Fluorescent Probe Melting Curve Analysis’, J Clin Pathol, 60 (2007), 1211-5.
122. G. Schindler, D. Capper, J. Meyer, W. Janzarik, H. Omran, C. Herold-Mende, K. Schmieder, P. Wesseling, C. Mawrin, M. Hasselblatt, D. N. Louis, A. Korshunov, S. Pfister, C. Hartmann, W. Paulus, G. Reifenberger, and A. von Deimling, ‘Analysis of Braf V600e Mutation in 1,320 Nervous System Tumors Reveals High Mutation Frequencies in Pleomorphic Xanthoastrocytoma, Ganglioglioma and Extra-Cerebellar Pilocytic Astrocytoma’, Acta Neuropathol, 121 (2011), 397-405.
123. S. Y. Sheu, F. Grabellus, S. Schwertheim, S. Handke, K. Worm, and K. W. Schmid, ‘Lack of Correlation between Braf V600e Mutational Status and the Expression Profile of a Distinct Set of Mirnas in Papillary Thyroid Carcinoma’, Horm Metab Res, 41 (2009), 482-7.
124. M. R. Stratton, P. J. Campbell, and P. A. Futreal, ‘The Cancer Genome’, Nature, 458 (2009), 719-24.
125. H. Torigoe, A. Ono, and T. Kozasa, ‘Hg(Ii) Ion Specifically Binds with T:T Mismatched Base Pair in Duplex DNA’, Chemistry, 16 (2010), 13218-25.
126. Y. Wang, D. Zheng, Q. Tan, M. X. Wang, and L. Q. Gu, ‘Nanopore-Based Detection of Circulating Micrornas in Lung Cancer Patients’, Nat Nanotechnol, 6 (2011), 668-74.
127. M. Yancovitz, A. Litterman, J. Yoon, E. Ng, R. L. Shapiro, R. S. Berman, A. C. Pavlick, F. Darvishian, P. Christos, M. Mazumdar, I. Osman, and D. Polsky, ‘Intra- and Inter-Tumor Heterogeneity of Braf(V600e)) Mutations in Primary and Metastatic Melanoma’, PLoS One, 7 (2012), e29336.

What is claimed is:

1. A method of detecting a thymine-thymine (T-T) base pair mismatch or a uracil-thymine (U-T) base pair mismatch in an at least partially double-stranded oligonucleotide (ds-oligonucleotide), the method comprising: reversibly binding Hg²⁺ to the base pair mismatch, thereby increasing the hybridization stability of the ds-oligonucleotide in comparison to its hybridization stability in the absence of Hg²⁺ reversible binding, wherein the T-T or U-T base pair mismatch is within a contiguous region of at least 10 nucleotides that are hybridized in the ds-oligonucleotide; and detecting the increased hybridization stability of the ds-oligonucleotide, thereby detecting the T-T or U-T base pair mismatch.

2. The method of claim 1, the method comprising: (a) hybridizing a first single-stranded oligonucleotide to a second single stranded oligonucleotide to form the at least partially ds-oligonucleotide comprising the T-T or U-T base pair mismatch and (b) contacting the ds-oligonucleotide with Hg²⁺.

3. The method of claim 2, wherein the Hg²⁺ is provided by the addition of HgCl₂.

4. The method of claim 2, wherein either the first single-stranded oligonucleotide or the second single-stranded oligonucleotide comprises a tag domain comprising a polydeoxycytosine covalently bound to the 3′-end, the 5′-end, or both the 3′-end and the 5′-end of the hybridizing region.

5. The method claim 4 wherein the tag domain is poly(dC)₃₀.

6. The method of claim 1, wherein at least 6, at least 7, at least 8, or at least 9 of the base-pairings within the contiguous hybridized region of at least 10 nucleotides are non-mismatched base-pairings.

7. The method of claim 1, wherein the base pair mismatch in the hybridized region is a thymine-thymine mismatch.

8. The method of claim 1, wherein the base pair mismatch in the hybridized region is a uracil-thymine mismatch.

9. The method of claim 1, wherein at least one of the first ss-oligonucleotide and the second ss-oligonucleotide comprises an oligonucleotide from about 10, 12, 14, 16, or 19 to about 20, 25, 30, 40, 50, 60, 100 or more nucleotides in length.

10. The method of claim 1, wherein the hybridized region is a contiguous region of between about 10, 12, 14, or 16 to about 20, 25, 30, 40, 50, 60, 100, or more nucleotides.

11. The method of claim 1, wherein the increase in hybridization stability of the ds-oligonucleotide is detected with a nanopore, PCR, gold nanoparticle, horseradish peroxidase, atomic force microscope, or immuo-PCR.

12. The method of claim 1, wherein the increased hybridization stability of the ds-oligonucleotide is detected with a nanopore.

13. The method of claim 12 wherein nanopore detection of the increase in hybridization stability of the ds-oligonucleotide comprises:
(a) applying a voltage to a sample containing the ds-oligonucleotide in a cis compartment of a duel chamber nanopore system, the voltage sufficient to drive translocation of the hybridized ds-oligonucleotide through a nanopore of said system by an unzipping process; and
(b) analyzing an electrical current pattern in the nanopore system over time, wherein the increased hybridization stability of the ds-oligonucleotide in the presence of reversible Hg²⁺ binding produces an electrical current pattern that is different and distinguishable from an electrical current pattern produced by the ds-oligonucleotide in the absence of Hg²⁺.

14. A method of determining whether a cytosine residue in a target single-stranded oligonucleotide (ss-oligonucleotide) or in a target strand of a double-stranded oligonucleotide (ds-oligonucleotide) is a methylated cytosine residue or an un-methylated cytosine residue, the method comprising:
(a) treating the target ss-oligonucleotide or target strand of the ds-oligonucleotide with bisulfite to convert an un-methylated cytosine residue, if present, to a uracil residue but wherein said treatment does not convert a methylated cytosine residue, if present, to a uracil residue;
(b) hybridizing the bisulfite treated target ss-oligonucleotide or bisulfite treated target strand of the ds-oligonucleotide and a probe molecule to form an at least partially double-stranded target/probe oligonucleotide that comprises a thymine residue base pair mismatched with the converted uracil residue, if present, from the target ss-oligonucleotide or target strand of the ds-oligonucleotide or that comprises a thymine residue base pair mismatched with the un-converted methylated cytosine residue, if present, from the target ss-oligonucleotide or target strand of the ds-oligonucleotide, wherein the uracil-thymine base pair mismatch or the methylated cytosine-thymine base pair mismatch is within a contiguous region of at least 10 nucleotides that are hybridized in the target/probe oligonucleotide;
(c) contacting the target/probe oligonucleotide with Hg²⁺, wherein Hg²⁺ reversibly binds the uracil-thymine base pair mismatch but not the methylated cytosine-thymine mismatch; and
(d) detecting the presence or absence of the reversible binding of Hg²⁺, wherein the presence indicates that the cytosine residue in the target ss-oligonucleotide or in the target strand of the ds-oligonucleotide was un-methylated and the absence indicates that the cytosine residue in the target ss-oligonucleotide or in the target strand of the ds-oligonucleotide was methylated.

15. The method of claim 14, wherein at least 6, at least 7, at least 8, or at least 9 of the base-pairings within the contiguous hybridized region of at least 10 nucleotides are non-mismatched base-pairings.

16. The method of claim 14, wherein at least the target ss-oligonucleotide or target strand of the ds-oligonucleotide, or probe molecule comprises an oligonucleotide from about 10, 12, 14, 16, or 19 to about 20, 25, 30, 40, 50, 60, 100 or more nucleotides in length.

17. The method of claim 14, wherein the probe molecule comprises a tag domain comprising a polydeoxycytosine covalently bound to the 3′-end, the 5′-end, or both the 3′-end and the 5′-end of the hybridizing region.

18. The method of claim 17 wherein the tag domain is poly(dC)₃₀.

19. The method of claim 14, wherein the Hg²⁺ is provided by the addition of HgCl₂.

20. The method of claim 14, the method further comprising detecting the increase in the hybridization stability of the target/probe oligonucleotide.

21. The method of claim 20, wherein the increase in hybridization stability of the target/probe oligonucleotide is detected with a nanopore, PCR, gold nanoparticle, horseradish peroxidase, atomic force microscope, or immuo-PCR.

22. The method of claim 20, wherein the increased hybridization stability of the ds-oligonucleotide is detected with a nanopore.

23. The method of claim 22, wherein the increase is detected using a nanopore, and the nanopore detection of the increase in hybridization stability of the ds-oligonucleotide comprises:
(a) applying a voltage to a sample containing the ds-oligonucleotide in a cis compartment of a duel chamber nanopore system, the voltage sufficient to drive translocation of the hybridized ds-oligonucleotide through a nanopore of said system by an unzipping process; and
(b) analyzing an electrical current pattern in the nanopore system over time, wherein the increased hybridization stability of the ds-oligonucleotide in the presence of reversible Hg²⁺ binding produces an electrical current pattern that is different and distinguishable from an electrical current pattern produced by the ds-oligonucleotide in the absence of Hg²⁺.

24. A method of increasing the hybridization stability of an at least partially double-stranded oligonucleotide (ds-oligonucleotide) comprising a thymine-thymine (T-T) base pair mismatch or a uracil-thymine (U-T) base pair mismatch, the method comprising: reversibly binding Hg²⁺ to the base pair mismatch, thereby increasing the hybridization stability of the ds-oligonucleotide, wherein the T-T or U-T base pair mismatch is within a contiguous region of at least 10 nucleotides that are hybridized in the ds-oligonucleotide.

25. The method of claim 24, the method comprising: (a) hybridizing a first single-stranded oligonucleotide to a second single stranded oligonucleotide to form the at least partially ds-oligonucleotide comprising the T-T or U-T base pair mismatch and (b) contacting the ds-oligonucleotide with Hg²⁺.

26. The method of claim 24, wherein at least 6, at least 7, at least 8, or at least 9 of the base-pairings within the contiguous hybridized region of at least 10 nucleotides are non-mismatched base-pairings.

27. The method of claim 24, wherein at least one of the first ss-oligonucleotide and the second ss-oligonucleotide comprises an oligonucleotide from about 10, 12, 14, 16, or 19 to about 20, 25, 30, 40, 50, 60, 100 or more nucleotides in length.

28. The method of claim 27, wherein either the first single-stranded oligonucleotide or the second single-stranded oligonucleotide comprises a tag domain comprising a polydeoxycytosine covalently bound to the 3′-end, the 5′-end, or both the 3′-end and the 5′-end of the hybridizing region.

29. The method claim 28, wherein the tag domain is poly(dC)₃₀.

30. The method of claim 24, wherein the Hg²⁺ is provided by the addition of HgCl₂.

31. The method of claim 24, the method further comprising detecting the increase in the hybridization stability of the target/probe oligonucleotide.

32. The method of claim 31, wherein the increase in hybridization stability of the target/probe oligonucleotide is detected with a nanopore, PCR, gold nanoparticle, horseradish peroxidase, atomic force microscope, or immuo-PCR.

33. The method of claim 31, wherein the increased hybridization stability of the ds-oligonucleotide is detected with a nanopore.

34. The method of claim 33, wherein the increase is detected using a nanopore, and the nanopore detection of the increase in hybridization stability of the ds-oligonucleotide comprises:
(a) applying a voltage to a sample containing the ds-oligonucleotide in a cis compartment of a duel chamber nanopore system, the voltage sufficient to drive translocation of the hybridized ds-oligonucleotide through a nanopore of said system by an unzipping process; and
(b) analyzing an electrical current pattern in the nanopore system over time, wherein the increased hybridization stability of the ds-oligonucleotide in the presence of reversible Hg²⁺ binding produces an electrical current pattern that is different and distinguishable from an electrical current pattern produced by the ds-oligonucleotide in the absence of Hg²⁺.

35. A method of detecting a cytosine-cytosine (C-C) base pair mismatch or a methylcytosine-cytosine (mC-C) base pair mismatch in an at least partially double-stranded oligonucleotide (ds-oligonucleotide), the method comprising: reversibly binding Ag⁺ to the base pair mismatch, thereby increasing the hybridization stability of the ds-oligonucleotide in comparison to its hybridization stability in the absence of Ag⁺ reversible binding, wherein the C-C base pair mismatch or mC-C base pair mismatch is within a contiguous region of at least 10 nucleotides that are hybridized in the ds-oligonucleotide; and detecting the increased hybridization stability of the ds-oligonucleotide thereby detecting the C-C base pair mismatch or mC-C base pair mismatch.

36. The method of claim 35, wherein the increase in hybridization stability of the ds-oligonucleotide is detected with a nanopore, PCR, gold nanoparticle, horseradish peroxidase, atomic force microscope, or immuo-PCR.

37. The method of claim 35, wherein the increased hybridization stability of the ds-oligonucleotide is detected with a nanopore.

38. The method claim 35 wherein at least 6, at least 7, at least 8, or at least 9 of the base-pairings within the contiguous hybridized region of at least 10 nucleotides are non-mismatched base-pairings.

39. The method of claim 35, the method comprising: (a) hybridizing a first single-stranded oligonucleotide to a second single stranded oligonucleotide to form the at least partially ds-oligonucleotide comprising the C-C or mC-C base pair mismatch and (b) contacting the ds-oligonucleotide with Ag⁺.

40. The method of claim 39, wherein either the first single-stranded oligonucleotide or the second single-stranded oligonucleotide comprises a tag domain comprising a polydeoxycytosine covalently bound to the 3′-end, the 5′-end, or both the 3′-end and the 5′-end of the hybridizing region.

41. The method claim 40 wherein the tag domain is poly(dC)₃₀.

42. The method of claim 35, wherein the base pair mismatch in the hybridized region is a cytosine-cytosine mismatch.

43. The method of claim 35, wherein the base pair mismatch in the hybridized region is a methylcytosine-cytosine mismatch.

44. The method of claim 39, wherein at least one of the first ss-oligonucleotide and the second ss-oligonucleotide comprises an oligonucleotide from about 10, 12, 14, 16, or 19 to about 20, 25, 30, 40, 50, 60, 100 or more nucleotides in length.

45. The method of claim 35, wherein the hybridized region is a contiguous region of between about 10, 12, 14, or 16 to about 20, 25, 30, 40, 50, 60, 100, or more nucleotides.

46. The method of claim 37 wherein nanopore detection of the increase in hybridization stability of the ds-oligonucleotide comprises:
(a) applying a voltage to a sample containing the ds-oligonucleotide in a cis compartment of a duel chamber nanopore system, the voltage sufficient to drive translocation of the hybridized ds-oligonucleotide through a nanopore of said system by an unzipping process; and
(b) analyzing an electrical current pattern in the nanopore system over time, wherein the increased hybridization stability of the ds-oligonucleotide in the presence of reversible Ag⁺ binding produces an electrical current pattern that is different and distinguishable from an electrical current pattern produced by the ds-oligonucleotide in the absence of Ag⁺.

47. A method of discriminating between a cytosine residue, a methylcytosine residue, and a hydroxymethylcytosine residue in a target single-stranded oligonucleotide (ss-oligonucleotide) or in a target strand of a double-stranded oligonucleotide (ds-oligonucleotide), the method comprising:
(a) hybridizing the target ss-oligonucleotide or target strand of the ds-oligonucleotide and a probe molecule to form an at least partially double-stranded target/probe oligonucleotide that comprises a cytosine residue from the probe molecule base pair mismatched with a cytosine from the target ss-oligonucleotide or target strand of the ds-oligonucleotide, if present, a cytosine residue from the probe molecule base pair mismatched with a methylcytosine residue from the target ss-oligonucleotide or target strand of the ds-oligonucleotide, if present, or a cytosine residue from the probe molecule base pair mismatched with a hydroxymethylcytosine residue from the target ss-oligonucleotide or target strand of the ds-oligonucleotide, if present, wherein the cytosine-cytosine mismatch, the cytosine-methylcytosine base pair mismatch, or the cytosine-hydroxymethylcytosine base pair mismatch is within a contiguous region of at least 10 nucleotides that are hybridized in the target/probe oligonucleotide;
(b) contacting the target/probe oligonucleotide with Ag⁺, wherein Ag⁺ reversibly binds the cytosine-cytosine base pair mismatch, the cytosine-methylcytosine base pair mismatch, and the cytosine-hydroxymethylcytosine base pair mismatch in a differential manner thus increasing the hybridization stability of the target/probe oligonucleotide in a differential manner depending on the presence of a cytosine-cytosine base pair mismatch, the cytosine-methylcytosine base pair mismatch, and the cytosine-hydroxymethylcytosine base pair mismatch; and
(c) detecting the reversible binding of Ag⁺ to the mismatch, wherein the amount of increase in the hybridization stability of the target/probe oligonucleotide discriminates whether the target ss-oligonucleotide or target strand of the ds-oligonucleotide contained a cytosine residue, a methylcytosine residue, or a hydroxymethylcytosine residue.

48. The method of claim 47, wherein at least 6, at least 7, at least 8, or at least 9 of the base-pairings within the contiguous hybridized region of at least 10 nucleotides are non-mismatched base-pairings.

49. The method of claim 47, wherein at least the target ss-oligonucleotide or target strand of the ds-oligonucleotide or probe molecule comprises an oligonucleotide from about 10, 12, 14, 16, or 19 to about 20, 25, 30, 40, 50, 60, 100 or more nucleotides in length.

50. The method of claim 47, wherein the probe molecule comprises a tag domain comprising a polydeoxycytosine covalently bound to the 3′-end, the 5′-end, or both the 3′-end and the 5′-end of the hybridizing region.

51. The method claim 50 wherein the tag domain is poly(dC)₃₀.

52. The method of claim 47, the method further comprising detecting the increase in the hybridization stability of the target/probe oligonucleotide.

53. The method of claim 52, wherein the increase in hybridization stability of the target/probe oligonucleotide is detected with a nanopore, PCR, gold nanoparticle, horseradish peroxidase, atomic force microscope, or immuo-PCR.

54. The method of claim 52, wherein the increased hybridization stability of the ds-oligonucleotide is detected with a nanopore.

55. The method of claim 54, wherein the increase is detected using a nanopore, and the nanopore detection of the increase in hybridization stability of the ds-oligonucleotide comprises:
(a) applying a voltage to a sample containing the ds-oligonucleotide in a cis compartment of a duel chamber nanopore system, the voltage sufficient to drive translocation of the hybridized ds-oligonucleotide through a nanopore of said system by an unzipping process; and
(b) analyzing an electrical current pattern in the nanopore system over time, wherein the increased hybridization stability of the ds-oligonucleotide in the presence of reversible Ag⁺ binding produces an electrical current pattern that is different and distinguishable from an electrical current pattern produced by the ds-oligonucleotide in the absence of Ag⁺.


SEQUENCE LISTING (33 sequences)

SEQ ID NO: 1 - DNA, 46 bp, Artificial Sequence
  Synthetic
tccctatcacgattatcccccccccccccccccccccccccccccc

SEQ ID NO: 2 - DNA, 16 bp, Artificial Sequence
  Synthetic
ataatcgtgttaggga

SEQ ID NO: 3 - DNA, 16 bp, Artificial Sequence
  Synthetic
ataatcgtgataggga

SEQ ID NO: 4 - DNA, 16 bp, Artificial Sequence
  Synthetic
ataatcgtgctaggga

SEQ ID NO: 5 - DNA, 16 bp, Artificial Sequence
  Synthetic
ataatcgtgutaggga

SEQ ID NO: 6 - DNA, 16 bp, Artificial Sequence
  Synthetic
ataatcgtgutaggga

SEQ ID NO: 7 - DNA, 16 bp, Artificial Sequence
  Synthetic
ataatcgtgctaggga

SEQ ID NO: 8 - DNA, 22 bp, Artificial Sequence
  Synthetic
gaatccgcgggagcgcggctgt

SEQ ID NO: 9 - DNA, 22 bp, Artificial Sequence
  Synthetic
gaatccgcgggagcgcggctgt

SEQ ID NO: 10 - DNA, 22 bp, Artificial Sequence
  Synthetic
gaatccgcgggagcgcggctgt

SEQ ID NO: 11 - DNA, 51 bp, Artificial Sequence
  Synthetic
acaacccccctccccctaattcccccccccccccccccccccccccccccc

SEQ ID NO: 12 - DNA, 51 bp, Artificial Sequence
  Synthetic
acaacccccctccctccaattcccccccccccccccccccccccccccccc

SEQ ID NO: 13 - DNA, 51 bp, Artificial Sequence
  Synthetic
acaacccctctccccccaattcccccccccccccccccccccccccccccc

SEQ ID NO: 14 - DNA, 51 bp, Artificial Sequence
  Synthetic
acaacctccctccccccaattcccccccccccccccccccccccccccccc

SEQ ID NO: 15 - DNA, 16 bp, Artificial Sequence
  Synthetic
aataaaatactataaa

SEQ ID NO: 16 - DNA, 16 bp, Artificial Sequence
  Synthetic
aataaaatactataaa

SEQ ID NO: 17 - DNA, 16 bp, Artificial Sequence
  Synthetic
aataaaatactataaa

SEQ ID NO: 18 - DNA, 16 bp, Artificial Sequence
  Synthetic
tttatactattttatt

SEQ ID NO: 19 - DNA, 44 bp, Artificial Sequence
  Synthetic
tttatactattttattagaaaaaaaaaagaaaaaaaaaaaaaaa

SEQ ID NO: 20 - DNA, 16 bp, Artificial Sequence
  Synthetic
ataatcgtgttaggga

SEQ ID NO: 21 - DNA, 16 bp, Artificial Sequence
  Synthetic
tccctatcacgattat

SEQ ID NO: 22 - DNA, 17 bp, Artificial Sequence
  Synthetic
tagctacagtgaaatct

SEQ ID NO: 23 - DNA, 17 bp, Artificial Sequence
  Synthetic
tagctacagagaaatct

SEQ ID NO: 24 - DNA, 47 bp, Artificial Sequence
  Synthetic
agatttctctgtagctacccccccccccccccccccccccccccccc

SEQ ID NO: 25 - DNA, 47 bp, Artificial Sequence
  Synthetic
atatttctctgtagctacccccccccccccccccccccccccccccc

SEQ ID NO: 26 - DNA, 47 bp, Artificial Sequence
  Synthetic
agatttatctgtagctacccccccccccccccccccccccccccccc

SEQ ID NO: 27 - DNA, 47 bp, Artificial Sequence
  Synthetic
agatttctctgtagcttcccccccccccccccccccccccccccccc

SEQ ID NO: 28 - DNA, 17 bp, Artificial Sequence
  Synthetic
cgagatttcactgtagc

SEQ ID NO: 29 - DNA, 17 bp, Artificial Sequence
  Synthetic
cgagatttctctgtagc

SEQ ID NO: 30 - DNA, 47 bp, Artificial Sequence
  Synthetic
gctacagtgaaatctcgcccccccccccccccccccccccccccccc

SEQ ID NO: 31 - DNA, 47 bp, Artificial Sequence
  Synthetic
gctacagtgaaatctatcccccccccccccccccccccccccccccc

SEQ ID NO: 32 - DNA, 47 bp, Artificial Sequence
  Synthetic
gctacattaaaatctcgcccccccccccccccccccccccccccccc

SEQ ID NO: 33 - DNA, 47 bp, Artificial Sequence
  Synthetic
gctacagtaaaatctctcccccccccccccccccccccccccccccc